(19) United States
(12) Patent Application Publication
(10) Pub. No.: US 2025/0221642 A1
(43) Pub. Date: Jul. 10, 2025

(54) POLYMERS WITH DUAL FUNCTIONALITY

(71) Applicant: DexCom, Inc., San Diego, CA (US)

(72) Inventors: Shane Richard Parnell, San Diego, CA (US); Shannon Reuben Woodruff, San Diego, CA (US); Devon M. Headen, San Diego, CA (US); Jiong Zou, San Diego, CA (US)

(73) Assignee: DexCom, Inc., San Diego, CA (US)

(21) Appl. No.: 19/007,839

(22) Filed: Jan. 2, 2025

Related U.S. Application Data
(60) Provisional application No. 63/618,186, filed on Jan. 5, 2024.

(51) Int. Cl. A61B 5/1486; A61B 5/145; C09D 175/04

(52) U.S. Cl. CPC A61B 5/1486; A61B 5/14532; A61B 5/14546; C09D 175/04

(57) ABSTRACT

Polymers with analyte diffusion resistance and interferent blocking functionality and improved layer uniformity for analyte monitoring are described. Methods of synthesizing such polymers, methods of providing analyte diffusion and interferent blocking and analyte monitoring systems comprising such polymers are provided.

### CROSS-REFERENCE TO RELATED APPLICATIONS

This application claims the benefit of U.S. Provisional Application No. 63/618,186, filed Jan. 5, 2024, which is incorporated by reference in its entirety.

### TECHNICAL FIELD

This disclosure is directed to polymers with analyte diffusion resistance and interferent blocking functionality and improved layer uniformity for analyte monitoring. Methods of synthesizing such polymers, methods of providing analyte diffusion and interferent blocking and analyte monitoring systems comprising such polymers are provided.

### BACKGROUND

Some current continuous analyte monitoring systems have a discrete interference layer (IL) that is designed to block interferents from reaching a working electrode. Such continuous analyte monitoring systems may also have a resistance layer (RL) which is designed to limit the flux of analyte to an enzyme layer. Some RL may also limit interferents from reaching the working electrode. An example of such RLs include blend of polymers, for example, a hydrophobic polyurethane with hydrophilic soft segments and a hydrophilic polymer such as polyvinyl pyrrolidone (PVP). Blending of polymers and their application to sensor constructs is challenging and introduces additional manufacturing steps.

### SUMMARY

In examples, a device for continuous measurement of a concentration of an analyte is provided, the device comprising a sensor configured to generate a signal associated with the concentration of the analyte, and a layer located over the sensor, the layer comprising a polyurethane, a poly(urea-urethane), or a poly(thiourethane-urethane), the polyurethane, the poly(urea-urethane), or the poly(thiourethane-urethane) comprising a repeating structure of: [{soft segment}-{hard segment}]; or [{soft segment}-{hard segment-chain extender-hard segment}]; and the soft segment comprises a backbone, the backbone having at least one polymer, at least one oligomer, or at least one monomer grafted thereto.

In aspects, the polyurethane, the poly(urea-urethane), or the poly(thiourethane-urethane), comprises the following Structure (I):

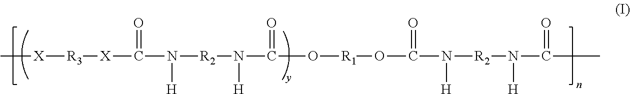

where [symbol] is the same as R1, where n is nonzero; where y is nonzero and represents a hard segment containing chain extender moles; where each X is, independently, oxygen, nitrogen, or sulfur; where R1 is a linear or branched alkyl, a polyether, a polyester, or a polycarbonate; R2 is a residue of an organic polyisocyanate; R3 is a linear aliphatic hydrocarbon, a non-linear aliphatic hydrocarbon, a cyclic aliphatic hydrocarbon, an aromatic hydrocarbon, or combinations thereof; and where at least one of R1 or R3 is represented by Structure (II):

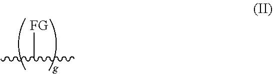

where g is nonzero; where FG is a functional group moiety that is the same or different when both R1 and R3 are represented by Structure (II).

In aspects, alone or in combination with any of the previous aspects, the at least one polymer, the at least one oligomer, or the at least one monomer is a zwitterionic moiety.

In aspects, alone or in combination with any of the previous aspects, the at least one polymer, the at least one oligomer, or the at least one monomer comprises a heterocycle. In aspects, alone or in combination with any of the previous aspects, the heterocycle comprises nitrogen. In aspects, alone or in combination with any of the previous aspects, the heterocycle comprises oxygen. In aspects, alone or in combination with any of the previous aspects, the heterocycle comprises nitrogen and oxygen.

In aspects, alone or in combination with any of the previous aspects, the heterocycle is at least one of furanyl, tetrahydrofuranyl, thiophenyl, pyrrolyl, pyrrolidinyl, pyranyl, pyridinyl, pyrazinyl, pyrimidinyl, pyrrolidonyl, piperidinyl, imidazolyl, thiazolyl, dioxanyl, morpholinyl, pyrimidinyl, isoxazolyl, oxazolyl, indolyl, isoindolyl, quinolinyl, isoquinolinyl, purinyl, carbazolyl, dibenzofuranyl, chromenyl, and xanthenyl.

In aspects, alone or in combination with any of the previous aspects, the functional group moiety is a grafted monomer selected from: N-vinylpyrrolidone monomer; vinyl pyridine monomer; vinyl pyrrole monomer, vinyl imidazole monomer, vinyl pyrimidine monomer, vinyl acetate monomer; acrylonitrile monomer; maleic anhydride monomer, poly(ethylene glycol) methyl ether methacrylate monomer; 2-(dimethylamino)ethyl methacrylate monomer; or combinations thereof.

In aspects, alone or in combination with any of the previous aspects, the polyurethane, the poly(urea-urethane), or the poly(thiourethane-urethane) is configured to control a flux of at least one analyte in vivo and configured to control a flux of at least one interferent in vivo.

In aspects, alone or in combination with any of the previous aspects, the layer is adjacent an electrode surface. In aspects, alone or in combination with any of the previous aspects, the layer is directly adjacent an electrode surface. In aspects, alone or in combination with any of the previous aspects, the layer is adjacent an enzyme-containing layer. In aspects, alone or in combination with any of the previous aspects, the layer is directly adjacent an enzyme-containing layer. In aspects, alone or in combination with any of the previous aspects, the layer is between an electrode surface and an enzyme-containing layer. In aspects, alone or in combination with any of the previous aspects, the layer is between an enzyme-containing layer and a biointerface layer.

In aspects, alone or in combination with any of the previous aspects, the analyte is glucose. In aspects, alone or in combination with any of the previous aspects, the analyte is a ketone. In aspects, alone or in combination with any of the previous aspects, the analyte is an alcohol, lactate, or potassium. In aspects, alone or in combination with any of the previous aspects, the interferent is acetaminophen or a metabolite thereof.

In other example, a method of attenuating a flux of an interferent and/or an analyte in an indwelling continuous electrochemical analyte sensor is provided, the method comprising providing a layer located over at least a portion of a sensor electrode configured to generate a signal associated with the concentration of the analyte, the layer comprising a polyurethane, a poly(urea-urethane), or a poly(thiourethane-urethane), the polyurethane, the poly(urea-urethane), or the poly(thiourethane-urethane) comprising a repeating structure of [{soft segment}-{hard segment}]; or [{soft segment}-{hard segment-chain extender-hard segment}]; wherein the soft segment comprises a backbone, the backbone having at least one polymer, at least one oligomer, or at least one monomer grafted thereto; and attenuating the flux of the interferent and/or the analyte through the layer.

In aspects, the at least one polymer, the at least one oligomer, or at least one monomer interacts with the interferent and/or the analyte resulting in the attenuating of the flux of the interferent and/or the analyte. In aspects, alone or in combination with any of the previous aspects, the at least one polymer, the at least one oligomer, or at least one monomer interacts with the interferent and/or the analyte via hydrogen bonding.

In aspects, alone or in combination with any of the previous aspects, the polyurethane, the poly(urea-urethane), or the poly(thiourethane-urethane), comprises the following Structure (I):

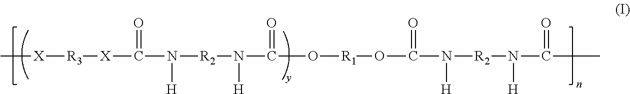

where [symbol] is the same as R1, where n is nonzero; where y is nonzero and represents a hard segment containing chain extender moles; where each X is, independently, oxygen, nitrogen, or sulfur; where R1 is a linear or branched alkyl, a polyether, a polyester, or a polycarbonate; R2 is a residue of an organic polyisocyanate; R3 is a linear aliphatic hydrocarbon, a non-linear aliphatic hydrocarbon, a cyclic aliphatic hydrocarbon, an aromatic hydrocarbon, or combinations thereof; and where at least one of R1 or R3 is represented by Structure (II):

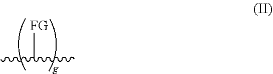

where g is nonzero; where FG is a functional group moiety that is the same or different when both R1 and R3 are represented by Structure (II).

In aspects, alone or in combination with any of the previous aspects, the polyurethane, the poly(urea-urethane), or the poly(thiourethane-urethane), comprises the following Structure (I):

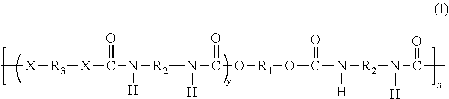

where y and n are non-zero; R1 is a functionalized α,ω-telechelic polymer or oligomer as described herein; R2 is a linear or branched alkyl, a polyether, a polyester, or a polycarbonate; R3 is an optional chain extender diol; X is, independently, nitrogen, oxygen, or sulfur.

In aspects, R1 is represented by Structure (Ill):

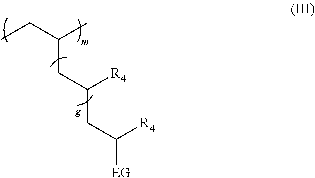

where m and g are nonzero; EG is H, an alkyl, aryl, benzyl, alkoxyl or hydrogen end group; R4 comprises furanyl, tetrahydrofuranyl, thiophenyl, pyrrolyl, pyrrolidinyl, pyranyl, pyridinyl, pyrazinyl, pyrimidinyl, pyrrolidonyl, piperidinyl, imidazolyl, thiazolyl, dioxanyl, morpholinyl, pyrimidinyl, isoxazolyl, oxazolyl, indolyl, isoindolyl, quinolinyl, isoquinolinyl, purinyl, carbazolyl, dibenzofuranyl, chromenyl, xanthenyl, or combinations thereof.

In aspects, R4 is grafted vinyl monomers, the vinyl monomer comprising a heterocycle. In aspects, alone or in combination with any of the previous aspects, the heterocycle comprises oxygen. In aspects, alone or in combination with any of the previous aspects, the heterocycle comprises nitrogen. In aspects, alone or in combination with any of the previous aspects, the heterocycle comprises nitrogen and oxygen.

In aspects, alone or in combination with any of the previous aspects, the heterocycle is at least one of furanyl, tetrahydrofuranyl, thiophenyl, pyrrolyl, pyrrolidinyl, pyranyl, pyridinyl, pyrazinyl, pyrimidinyl, pyrrolidonyl, piperidinyl, imidazolyl, thiazolyl, dioxanyl, morpholinyl, pyrimidinyl, isoxazolyl, oxazolyl, indolyl, isoindolyl, quinolinyl, isoquinolinyl, purinyl, carbazolyl, dibenzofuranyl, chromenyl, and xanthenyl.

In aspects, alone or in combination with any of the previous aspects, the grafted vinyl monomer is selected from: N-vinylpyrrolidone monomer; vinyl pyridine monomer; vinyl pyrrole monomer, vinyl imidazole monomer, vinyl pyrimidine monomer, vinyl acetate monomer; acrylonitrile monomer; maleic anhydride monomer, poly(ethylene glycol) methyl ether methacrylate monomer; 2-(dimethylamino)ethyl methacrylate monomer; or combinations thereof.

In aspects, the vinyl monomers are selected from the group:

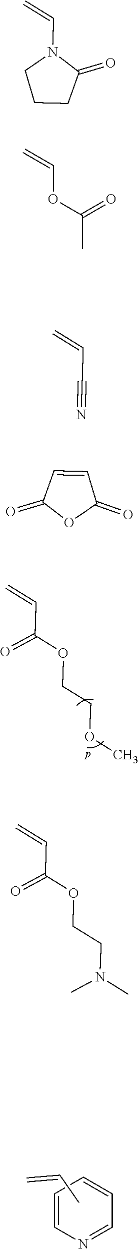

where p is non-zero.

In aspects, alone or in combination with any of the previous aspects, the layer is adjacent a surface of the sensor electrode. In aspects, alone or in combination with any of the previous aspects, the layer is directly adjacent a surface of the sensor electrode.

In another aspect, R1 is a zwitterionic functionalized α,ω-telechelic polymer represented by Structure (IV):

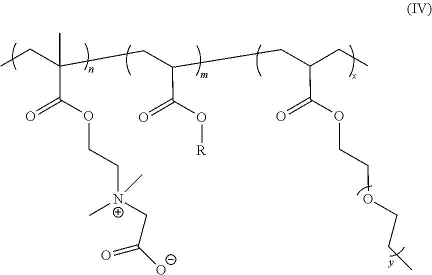

where n is nonzero; m, x and y are independently zero or nonzero; R is a straight chain or branched chain alkyl, aryl, benzyl, alkoxyl or hydrogen. In examples, x and y are nonzero comprise polyethylene glycol units, for example, to provide or to adjust miscibility. Repeat units n, m, and y monomer ratios can be adjusted to reach a ratio miscible with other soft segments and/or to improve TPU solubility in organic solvents.

In examples, zwitterionic functionalized α,ω-telechelic polymer are employed as soft segments and/or copolymerized with other soft segments with polyisocyanate to form prepolymers and then chain extend to provide zwitterionic IL-RL TPUs.

In aspects, alone or in combination with any of the previous aspects, the layer is adjacent an enzyme layer of the indwelling continuous electrochemical analyte sensor. In aspects, alone or in combination with any of the previous aspects, the layer is directly adjacent an enzyme layer of the indwelling continuous electrochemical analyte sensor.

In aspects, alone or in combination with any of the previous aspects, the layer is adjacent a biointerface layer of the indwelling continuous electrochemical analyte sensor. In aspects, alone or in combination with any of the previous aspects, the layer is directly adjacent a biointerface layer of the indwelling continuous electrochemical analyte sensor.

In aspects, alone or in combination with any of the previous aspects, the analyte is glucose. In aspects, alone or in combination with any of the previous aspects, the analyte is a ketone. In aspects, alone or in combination with any of the previous aspects, the analyte is glucose and a ketone. In aspects, alone or in combination with any of the previous aspects, the interferent is acetaminophen or a metabolite thereof. In aspects, alone or in combination with any of the previous aspects, the analyte is glucose and the interferent is acetaminophen or a metabolite thereof. In aspects, alone or in combination with any of the previous aspects, the interferent is acetaminophen or a metabolite thereof. In aspects, alone or in combination with any of the previous aspects, the analyte is ketone and the interferent is acetaminophen or a metabolite thereof.

A device for measurement of a concentration of an analyte, the device comprising a transcutaneous sensor configured to generate a signal associated with the concentration of the analyte in vivo; and a membrane located over the sensor, wherein the membrane comprises a polyurethane containing block copolymer, wherein the polyurethane containing block copolymer has at least one hard segment and at least one soft segment, the at least one soft segment comprises a backbone, the backbone having at least one function group moiety covalently attached thereto, wherein the membrane is configured to control a flux of at least one analyte in vivo, configured to control a flux of at least one analyte in vivo, or both. In aspects, the at least one soft segment comprises a polycarbonate-urethane, polyether-urethane, and polyester-urethane.

In aspects, alone or in combination with any of the previous aspects, the at least one functional group moiety is selected from furanyl, tetrahydrofuranyl, thiophenyl, pyrrolyl, pyrrolidinyl, pyranyl, pyridinyl, pyrazinyl, pyrimidinyl, pyrrolidonyl, piperidinyl, imidazolyl, thiazolyl, dioxanyl, morpholinyl, pyrimidinyl, isoxazolyl, oxazolyl, indolyl, isoindolyl, quinolinyl, isoquinolinyl, purinyl, carbazolyl, dibenzofuranyl, chromenyl, and xanthenyl;

In aspects, alone or in combination with any of the previous aspects, the at least one functional group moiety is a grafted monomer selected from: N-vinylpyrrolidone monomer; 2-vinyl pyridine monomer; 4-vinyl pyridine monomer; vinyl pyrrole monomer, vinyl imidazole monomer, vinyl pyrimidine monomer, vinyl acetate monomer; acrylonitrile monomer; maleic anhydride monomer, poly(ethylene glycol) methyl ether methacrylate monomer; 2-(dimethylamino)ethyl methacrylate monomer; or combinations thereof.

In another example, a device for measurement of a concentration of an analyte is provided, the device comprising a sensor configured to generate a signal associated with the concentration of the analyte; and a membrane located over the sensor, the membrane configured to control a flux of the analyte therethrough, the membrane further configured to control a flux of one or more interferents therethrough, the membrane comprising a polymer comprising both hydrophilic and hydrophobic segments. In aspects, alone or in combination with any of the previous aspects, the polymer comprises a hard segment and a soft segment. In aspects, alone or in combination with any of the previous aspects, the polymer comprises both hydrophilic and hydrophobic segments, a soft segment, and a hard segment.

In aspects, the membrane comprises a polyurethane containing block copolymer. In aspects, alone or in combination with any of the previous aspects, the polyurethane containing block copolymer is chosen from a polycarbonate-urethane, polyether-urethane, and polyester-urethane.

In aspects, alone or in combination with any of the previous aspects, the polyurethane containing block copolymer comprises at least one hard segment and at least one soft segment. In aspects, alone or in combination with any of the previous aspects, at least a portion of the at least one hard segment is hydrophobic. In aspects, alone or in combination with any of the previous aspects, at least a portion of the at least one soft segment comprises a functionalized α,ω-telechelic polymer or oligomer. In aspects, alone or in combination with any of the previous aspects, at least a portion of the at least one soft segment comprises a polyalkyl. In aspects, alone or in combination with any of the previous aspects, at least a portion of the at least one soft segment comprises a polyalkyl. In aspects, alone or in combination with any of the previous aspects, at least a portion of the at least one soft segment comprises a polyalkylglycol. In aspects, alone or in combination with any of the previous aspects, at least a portion of the at least one soft segment comprises a polyalkylglycol and a α,ω-telechelic polymer or oligomer.

In aspects, alone or in combination with any of the previous aspects, the at least one soft segment has a backbone grafted with at least one functional group containing moiety. In aspects, alone or in combination with any of the previous aspects, the at least one functional group containing moiety has a linear backbone and a terminus. In aspects, alone or in combination with any of the previous aspects, at least one functional group of the functional group containing moiety is present at the terminus of the graft.

In aspects, alone or in combination with any of the previous aspects, the at least one functional group of the functional group containing moiety is present along the linear backbone. In aspects, alone or in combination with any of the previous aspects, the at least one functional group of the functional group containing moiety is present along the linear backbone and at the terminus.

In aspects, alone or in combination with any of the previous aspects, the at least one functional group containing moiety comprises one or more of: a zwitterion, furanyl, tetrahydrofuranyl, thiophenyl, pyrrolyl, pyrrolidinyl, pyranyl, pyridinyl, pyrazinyl, pyrimidinyl, pyrrolidonyl, piperidinyl, imidazolyl, thiazolyl, dioxanyl, morpholinyl, pyrimidinyl, isoxazolyl, oxazolyl, indolyl, isoindolyl, quinolinyl, isoquinolinyl, purinyl, carbazolyl, dibenzofuranyl, chromenyl, and xanthenyl.

In aspects, alone or in combination with any of the previous aspects, the covalently attached moiety is a grafted monomer selected from: N-vinylpyrrolidone monomer; vinyl pyridine monomer; vinyl pyrrole monomer, vinyl imidazole monomer, vinyl pyrimidine monomer, vinyl acetate monomer; acrylonitrile monomer; maleic anhydride monomer, poly(ethylene glycol) methyl ether methacrylate monomer; 2-(dimethylamino)ethyl methacrylate monomer; or combinations thereof.

In another example, an in vivo interferent blocking intermediate for incorporation within a polyurethane or polyurethane-urea is provided, the in vivo interferent blocking intermediate having the structure (II):

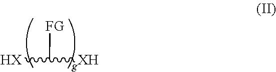

where [symbol] is linear or branched alkyl, polyether, polyester, or polycarbonate; where g is nonzero; where each X is, independently, oxygen, nitrogen, or sulfur; where FG is a covalently attached functional group moiety; and H is hydrogen.

In aspects, the covalently attached moiety comprises a hydrogen bonding acceptor group. In aspects, alone or in combination with any of the previous aspects, the hydrogen bonding acceptor group comprises a heterocycle.

In aspects, alone or in combination with any of the previous aspects, the covalently attached moiety is a grafted monomer selected from: a zwitterionic moiety, N-vinylpyrrolidone monomer; vinyl pyridine monomer; vinyl pyrrole monomer, vinyl imidazole monomer, vinyl pyrimidine monomer, vinyl acetate monomer; acrylonitrile monomer; maleic anhydride monomer, poly(ethylene glycol) methyl ether methacrylate monomer; 2-(dimethylamino)ethyl methacrylate monomer; or combinations thereof.

In aspects, alone or in combination with any of the previous aspects, the interferent blocking intermediate has an average molecular weight of at least 100 daltons to less than 10,000 daltons.

In aspects, alone or in combination with any of the previous aspects, [symbol] is α,ω-homotelechelic polymer or oligomer. In aspects, alone or in combination with any of the previous aspects, the α,ω-homotelechelic polymer or oligomer is a di-hydroxyl polymer or di-hydroxyl block copolymer. In aspects, alone or in combination with any of the previous aspects, the α,ω-homotelechelic polymer or oligomer is a di-halide polymer or di-halide block copolymer. In aspects, alone or in combination with any of the previous aspects, the α,ω-homotelechelic polymer or oligomer is a di-thiol polymer or a di-thiol block copolymer. In aspects, alone or in combination with any of the previous aspects, the α,ω-homotelechelic polymer or oligomer is a di-amine polymer or a di-amine block copolymer.

In another example, a process for preparing a polymer configured for in vivo interferent blocking and/or in vivo control of a flux of at least one analyte is provided, the process comprising: grafting of at least one monomer to a α,ω telechelic polymer or oligomer using at least one of: atom transfer radical polymerization (ATRP); Nitroxide-mediated polymerization (NMP); Reversible addition-fragmentation chain transfer (RAFT) polymerization; Reversible addition-fragmentation chain transfer (RAFT) polymerization combined with ring-opening metathesis polymerization (ROMP); or Iodine-transfer polymerization (ITP), so as to form a functionalized α, ω telechelic intermediate; reacting the functionalized α, ω telechelic intermediate with a diisocyanate; and forming a polyurethane block copolymer having at least one soft segment comprising the functionalized α, ω telechelic intermediate, the soft segment configured for in vivo interferent blocking, in vivo control of a flux of at least one analyte, or both.

In aspects, the at least one monomer is grafted to a backbone of the α, ω telechelic polymer or oligomer. In aspects, alone or in combination with any of the previous aspects, the at least one monomer is a zwitterionic moiety, N-vinylpyrrolidone, vinyl pyridine; vinyl pyrrole, vinyl imidazole, vinyl pyrimidine, vinyl acetate; acrylonitrile; maleic anhydride, poly(ethylene glycol) methyl ether methacrylate; 2-(dimethylamino)ethyl methacrylate; or combinations thereof.

In aspects, alone or in combination with any of the previous aspects, the at least one monomer comprises a vinyl group. In aspects, alone or in combination with any of the previous aspects, the vinyl group further comprises a zwitterionic moiety, furanyl, tetrahydrofuranyl, thiophenyl, pyrrolyl, pyrrolidinyl, pyranyl, pyridinyl, pyrazinyl, pyrimidinyl, pyrrolidonyl, piperidinyl, imidazolyl, thiazolyl, dioxanyl, morpholinyl, pyrimidinyl, isoxazolyl, oxazolyl, indolyl, isoindolyl, quinolinyl, isoquinolinyl, purinyl, carbazolyl, dibenzofuranyl, chromenyl, and xanthenyl.

In aspects, alone or in combination with any of the previous aspects, the polyisocyanate is selected from one or more of norbornane diisocyanate (NBDI), isophorone diisocyanate (IPDI), toluene diisocyanate (TDI), 1,3-phenylene diisocyanate (MPDI), trans-1,3-bis(isocyanatomethyl) cyclohexane (1,3-H6XDI), bicyclohexylmethane-4,4′-diisocyanate (HMDI), 4,4′-Diphenylmethane diisocyanate (MDI), trans-1,4-bis(isocyanatomethyl) cyclohexane (1,4-H6XDI), 1,4-cyclohexyl diisocyanate (CHDI), 1,4-phenylene diisocyanate (PPDI), 3,3′-Dimethyl-4,4′-biphenyldiisocyanate (TODI), 1,6-hexamethylene diisocyanate (HDI), or combinations thereof.

In aspects, alone or in combination with any of the previous aspects, at least a portion of the at least one soft segment comprises a polyether. In aspects, alone or in combination with any of the previous aspects, at least a portion of the at least one soft segment comprises polycarbonate.

In aspects, alone or in combination with any of the previous aspects, the α, ω telechelic intermediate is a polymer or block copolymer with a α, ω-terminus selected from hydroxyl, amine, thiol, halide, or combinations thereof.

In another example a polymer is provided, the polymer having the following Structure (I):

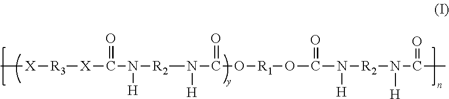

where [symbol] is the same as R1, where g is nonzero; where n is nonzero; where y is nonzero and represents a hard segment containing chain extender moles; where each X is, independently, oxygen, nitrogen, or sulfur; where R1 is a linear or branched alkyl, a polyether, a polyester, or a polycarbonate; R2 is a residue of an organic polyisocyanate; R3 is a linear aliphatic hydrocarbon, a non-linear aliphatic hydrocarbon, a cyclic aliphatic hydrocarbon, an aromatic hydrocarbon, or combinations thereof; and where at least one of R1 or R3 is represented by Structure (II):

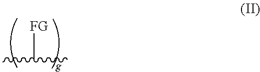

where FG is a functional group moiety that is the same or different when both R1 and R3 are represented by Structure (II).

In aspects, n, y, and z are nonzero. In aspects, alone or in combination with any of the previous aspects, X is, independently, oxygen or nitrogen. In aspects, alone or in combination with any of the previous aspects, X is oxygen. In aspects, alone or in combination with any of the previous aspects, [symbol] comprises a polyalkyl, polyalkylglycol, or combinations thereof.

In aspects, alone or in combination with any of the previous aspects, the functional group moiety FG is a grafted monomer selected from: N-vinylpyrrolidone monomer; vinyl pyridine monomer; vinyl pyrrole monomer, vinyl imidazole monomer, vinyl pyrimidine monomer, vinyl acetate monomer; acrylonitrile monomer; maleic anhydride monomer, poly(ethylene glycol) methyl ether methacrylate monomer; 2-(dimethylamino)ethyl methacrylate monomer; or combinations thereof.

In aspects, alone or in combination with any of the previous aspects, the covalently attached moiety comprises furanyl, tetrahydrofuranyl, thiophenyl, pyrrolyl, pyrrolidinyl, pyranyl, pyridinyl, pyrazinyl, pyrimidinyl, pyrrolidonyl, piperidinyl, imidazolyl, thiazolyl, dioxanyl, morpholinyl, pyrimidinyl, isoxazolyl, oxazolyl, indolyl, isoindolyl, quinolinyl, isoquinolinyl, purinyl, carbazolyl, dibenzofuranyl, chromenyl, and xanthenyl.

### BRIEF DESCRIPTION OF THE DRAWINGS

In order to understand and to see how the present disclosure may be carried out in practice, examples will now be described, by way of non-limiting examples only, with reference to the accompanying drawings, in which:

FIG. 1 is a perspective view of an exemplary example of a continuous analyte sensor.

FIGS. 2A-2C are cross-sectional views through the sensor of FIG. 1 on line 2-2, illustrating various examples of the membrane system.

FIG. 2D is a sectional view of an exemplary planar continuous analyte sensor.

FIG. 3A is a side view schematic illustrating an in vivo portion of a continuous analyte sensor, in examples.

FIG. 3B is a perspective view schematic illustrating an in vivo portion of a continuous analyte sensor, in examples.

FIG. 3C is a side view schematic illustrating an in vivo portion of a continuous analyte sensor, in examples.

FIG. 4 is a representation of an exemplary polymer with hard segments and soft segments in accordance with various technologies described in the present disclosure.

FIGS. 5A, 5B, 5C are representations of functionalized α,ω-telechelic polymer/oligomer containing polymer having hydrogen bond acceptor groups interacting with interferents having hydrogen donor groups.

FIG. 6A is a diagram illustrating an example synthesis pathway for functionalizing a α,ω-telechelic polymer/oligomer and subsequent polyurethane synthesis using the functionalized α,ω-telechelic polymer/oligomer in accordance with various technologies described in the present disclosure.

FIG. 6B is a diagram illustrating an example synthesis pathway for providing various polyurethanes or polyurethane ureas or poly(thiourethane) ureas using the functionalized α,ω-telechelic polymer/oligomer in accordance with various technologies described in the present disclosure.

FIG. 7 is a diagram illustrating an example continuous analyte monitoring sensor system communicating with at least one display device in accordance with various technologies described in the present disclosure.

### DETAILED DESCRIPTION

The following description and figures illustrate examples of the present disclosure in detail. Those of skill in the art will recognize that there are numerous variations and modifications of this disclosure that are encompassed by its scope. Accordingly, the description examples herein should not be deemed to limit the scope of the present disclosure. In order to facilitate an understanding of the examples disclosed herein, a number of terms are defined below.

Chemical structures used herein are to be understood to be represented by conventional atom labels and when shown as a branched or unbranched “chain” of non-zero length “a” are to be understood as a hydrocarbon chain of methylene and/or methine groups, for example

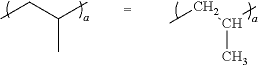

The term “about” as used herein is a broad term, and is to be given its ordinary and customary meaning to a person of ordinary skill in the art (and is not to be limited to a special or customized meaning), and refers without limitation to allowing for a degree of variability in a value or range, for example, within 10%, within 5%, or within 1% of a stated value or of a stated limit of a range, and includes the exact stated value or range.

The term “adhere” and “attach” as used herein are broad terms, and are to be given their ordinary and customary meaning to a person of ordinary skill in the art (and are not to be limited to a special or customized meaning), and refer without limitation to hold, bind, or stick, for example, by gluing, bonding, grasping, interpenetrating, or fusing.

The term “analyte” as used herein is a broad term, and is to be given its ordinary and customary meaning to a person of ordinary skill in the art (and is not to be limited to a special or customized meaning), and refers without limitation to a substance or chemical constituent in a biological fluid (e.g., blood, interstitial fluid, cerebral spinal fluid, lymph fluid, urine, sweat, saliva, etc.) that can be analyzed. Analytes can include naturally occurring substances, artificial substances, metabolites, and/or reaction products. In some examples, the analyte measured by the sensing regions, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); bilirubin, biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine; creatine kinase; creatine kinase MM isoenzyme; creatinine; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycerol; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; beta-hydroxybutyrate; ketones; lactate; lead; lipoproteins ((a), B/A-1, β); lysozyme; mefloquine; netilmicin; oxygen; phenobarbitone; phenytoin; phytanic/pristanic acid; potassium, sodium, and/or other blood electrolytes; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uric acid; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain examples. The analyte can be naturally present in the biological fluid, or endogenous, for example, a metabolic product, a hormone, an antigen, an antibody, and the like. Alternately, the analyte can be introduced into the body, or exogenous, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, RITALIN®, CYLERT®, PRELUDIN®, DIDREX®, PRESTATE®, VORANIL®, SANDREX®, PLEGINE®); depressants (barbiturates, methaqualone, tranquilizers such as VALIUM®, LIBRIUM®, MILTOWN®, SERAX®, EQUANIL®, TRANXENE®); hallucinogens (phencyclidine, lysergic acid, mescaline, peyote, psilocybin); narcotics (heroin, codeine, morphine, opium, meperidine, PERCOCET®, PERCODAN®, TUSSIONEX®, fentanyl, DARVON®, TALWIN®, LOMOTIL®); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC®), homovanillic acid (HVA), 5-hydroxytryptamine (5HT), 5-hydroxyindoleacetic acid (FHIAA), and histamine.

The phrases “analyte-measuring device,” “analyte-monitoring device,” “analyte-sensing device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases may refer without limitation to an instrument responsible for detection of a particular analyte or combination of analytes. In examples, the instrument includes a sensor coupled to circuitry disposed within a housing, and configure to process signals associated with analyte concentrations into information. In examples, such apparatuses and/or systems are capable of providing specific quantitative, semi-quantitative, qualitative, and/or semi qualitative analytical information using a biological recognition element combined with a transducing (detecting) element.

The term “amphiphilic” as used herein is a broad term and is to be given its ordinary and customary meaning to a person of ordinary skill in the art (and are not to be limited to a special or customized meaning), and refer without limitation to a chemical compound or polymer possessing both hydrophilic and hydrophobic segments or properties.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, and/or multi-analyte sensor device responsible for the detection of, or transduction of a signal associated with, a particular analyte or combination of analytes. In examples, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In examples, such biosensors and/or sensors are capable of providing specific quantitative, semi-quantitative, qualitative, semi qualitative analytical signals using a biological recognition element combined with a transducing (detecting) element.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In examples, the sensing portion, sensing membrane, and/or sensing mechanism generally comprise an electrode configured to provide signals during electrochemically reactions with one or more membranes covering electrochemically reactive surface. In examples, such sensing portions, sensing membranes, and/or sensing mechanisms can provide specific quantitative, semi-quantitative, qualitative, semi qualitative analytical signals using a biological recognition element combined with a transducing (detecting) element.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The phrase “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “adhere” and “attach” as used herein are broad terms, and are to be given their ordinary and customary meaning to a person of ordinary skill in the art (and are not to be limited to a special or customized meaning), and refer without limitation to hold, bind, or stick, for example, by gluing, bonding, grasping, interpenetrating, or fusing.

The phrase “barrier cell layer” as used herein is a broad phrase, and is to be given its ordinary and customary meaning to a person of ordinary skill in the art (and is not to be limited to a special or customized meaning), and refers without limitation to a part of a foreign body response that forms a cohesive monolayer of cells (for example, macrophages and foreign body giant cells) that substantially block the transport of molecules and other substances to the implantable device.

The term “bioactive agent” as used herein is a broad term, and is to be given its ordinary and customary meaning to a person of ordinary skill in the art (and is not to be limited to a special or customized meaning), and refers without limitation to any substance that has an effect on or elicits a response from living tissue.

The phrases “biointerface membrane” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The term “biostable” as used herein is a broad term, and is to be given its ordinary and customary meaning to a person of ordinary skill in the art (and is not to be limited to a special or customized meaning), and refers without limitation to materials that are relatively resistant to degradation by processes that are encountered in vivo.

The phrase “cell processes” as used herein is a broad phrase, and is to be given its ordinary and customary meaning to a person of ordinary skill in the art (and is not to be limited to a special or customized meaning), and refers without limitation to pseudopodia of a cell.

The phrase “cellular attachment” as used herein is a broad phrase, and is to be given its ordinary and customary meaning to a person of ordinary skill in the art (and is not to be limited to a special or customized meaning), and refers without limitation to adhesion of cells and/or cell processes to a material at the molecular level, and/or attachment of cells and/or cell processes to microporous material surfaces or macroporous material surfaces. One example of a material used in the prior art that encourages cellular attachment to its porous surfaces is the BIOPORE™ cell culture support marketed by Millipore (Bedford, Mass.), and as described in Brauker et al., U.S. Pat. No. 5,741,330.

The term “cofactor” as used herein is a broad term, and is to be given its ordinary and customary meaning to a person of ordinary skill in the art (and is not to be limited to a special or customized meaning), and refers without limitation to one or more substances whose presence contributes to or is required for analyte-related activity of an enzyme. Analyte-related activity can include, but is not limited to, any one of or a combination of binding, electron transfer, and chemical transformation. Cofactors are inclusive of coenzymes, non-protein chemical compounds, metal ions and/or metal organic complexes. Coenzymes are inclusive of prosthetic groups and co-substrates.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrase “continuous analyte sensing” as used herein is a broad phrase, and is to be given their ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every second or less to about one week or more. In further examples, monitoring of analyte concentration is performed from about every 2, 3, 5, 7, 10, 15, 20, 25, 30, 35, 40, 45, 50, 55, or 60 seconds to about every 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, monitoring of analyte concentration is performed from about 10, 20, 30, 40 or 50 minutes to about every 1, 2, 3, 4, 5, 6, 7 or 8 hours. In further examples, monitoring of analyte concentration is performed from about every 8 hours to about every 12, 16, 20, or 24 hours. In further examples, monitoring of analyte concentration is performed from about every day to about every 1.5, 2, 3, 4, 5, 6, or 7 days. In further examples, monitoring of analyte concentration is performed from about every week to about every 1.5, 2, 3 or more weeks. The phrase “continuous analyte sensing” as used herein is inclusive of “continuous multi-analyte sensing”, where two or more analytes are continuously, continually, and/or intermittently (but regularly) performed monitored, either of the two or more analytes being monitored concurrently or non-concurrently.

The term “coaxial” as used herein is to be construed broadly to include sensor architectures having elements aligned along a shared axis around a core that can be configured to have a circular, elliptical, triangular, polygonal, or other cross-section such elements can include electrodes, insulating layers, or other elements that can be positioned circumferentially around the core layer, such as a core electrode or core polymer wire.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. For example, an element is “coupled” if the element is covalently, communicatively, electrostatically, thermally connected, mechanically connected, magnetically connected, or ionically associated with, or physically entrapped, adsorbed to or absorbed by another element. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another as in covalently, electrostatically, mechanically, thermally, magnetically, ionically associated with, or physically entrapped, or absorbed (i.e. “directly coupled” as in no intervening element(s)). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components. covalently, electrostatically, ionically associated with, or physically entrapped, or absorbed.

The phrase “defined edges” as used herein is a broad phrase, and is to be given its ordinary and customary meaning to a person of ordinary skill in the art (and is not to be limited to a special or customized meaning), and refers without limitation to abrupt, distinct edges or borders among layers, domains, coatings, or portions. “Defined edges” are in contrast to a gradual transition between layers, domains, coatings, or portions.

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of a membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “drift” as used herein is a broad term, and is to be given its ordinary and customary meaning to a person of ordinary skill in the art (and is not to be limited to a special or customized meaning), and refers without limitation to a progressive increase or decrease in signal over time that is unrelated to changes in host systemic analyte concentrations (e.g., host postprandial glucose concentrations). While not wishing to be bound by theory, it is believed that drift may be the result of a local decrease in glucose transport to the sensor, for example, due to formation of a foreign body capsule (FBC), or due to an insufficient amount of interstitial fluid surrounding the sensor, which results in reduced oxygen and/or glucose transport to the sensor. In examples, an increase in local interstitial fluid may slow or reduce drift and thus improve sensor performance. Drift may also be the result of sensor electronics, or algorithmic models used to compensate for noise or other anomalies that can occur with electrical signals in, for example, the picoAmp range, the femtoAmp range, the nanoAmp range, the microAmp range, the milliAmp range, the Amp range, etc.

The phrases “drug releasing membrane” and “drug releasing layer” as used interchangeably herein are each a broad phrase, and each are to be given its ordinary and customary meaning to a person of ordinary skill in the art (and is not to be limited to a special or customized meaning), and refers without limitation to a permeable or semi-permeable membrane which is permeable to one or more bioactive agents. In examples, the “drug releasing membrane” and “drug releasing layer” is typically of a few microns thickness or more and can be comprised of two or more domains. In examples the drug releasing layer and/or drug releasing membrane are substantially the same as the biointerface layer and/or biointerface membrane. In another example, the drug releasing layer and/or drug releasing membrane are distinct from the biointerface layer and/or biointerface membrane.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In examples this reaction is faradaic and results in charge transfer between the surface and its environment. In examples, hydrogen peroxide produced by an enzyme-catalyzed reaction of an analyte being oxidized on the surface results in a measurable electronic current. For example, in the detection of glucose, glucose oxidase produces hydrogen peroxide (H₂O₂) as a byproduct. The H₂O₂ reacts with the surface of the working electrode to produce two protons (2H⁺), two electrons (2e⁻) and one molecule of oxygen (O₂), which produces the electronic current being detected. In a counter electrode, a reducible species, for example, O₂ is reduced at the electrode surface so as to balance the current generated by the working electrode.

The term “electrolysis” as used herein is a broad term, and is to be given its ordinary and customary meaning to a person of ordinary skill in the art (and is not to be limited to a special or customized meeting), and refers without limitation to electrooxidation or electroreduction (collectively, “redox”) of a compound, either directly or indirectly, by one or more enzymes, cofactors, or mediators.

The phrase “hard segment” as used herein is a broad phrase, and is to be given its ordinary and customary meaning to a person of ordinary skill in the art (and is not to be limited to a special or customized meaning), and refers without limitation to an element of a copolymer, for example, a polyurethane, a polycarbonate polyurethane, or a polyurethane urea copolymer, which imparts resistance properties, e.g., resistance to bending or twisting. The phrase “hard segment” can be further characterized as a crystalline, semi-crystalline, or glassy material with a glass transition temperature determined by dynamic scanning calorimetry (“Tg”) typically above ambient temperature. Exemplary hard segment elements used to prepare a polycarbonate polyurethane, or a polyurethane urea hard segment include norbornane diisocyanate (NBDI), isophorone diisocyanate (IPDI), toluene diisocyanate (TDI), 1,3-phenylene diisocyanate (MPDI), trans-1,3-bis(isocyanatomethyl) cyclohexane (1,3-H6XDI), bicyclohexylmethane-4,4′-diisocyanate (HMDI), 4,4′-Diphenylmethane diisocyanate (MDI), trans-1,4-bis(isocyanatomethyl) cyclohexane (1,4-H6XDI), 1,4-cyclohexyl diisocyanate (CHDI), 1,4-phenylene diisocyanate (PPDI), 3,3′-Dimethyl-4,4′-biphenyldiisocyanate (TODI), 1,6-hexamethylene diisocyanate (HDI), or combinations thereof.

The term “host” as used herein is a broad term, and is to be given its ordinary and customary meaning to a person of ordinary skill in the art (and is not to be limited to a special or customized meaning), and refers without limitation to mammals, for example humans.

The terms “indwelling,” “in dwelling,” “implanted,” or “implantable” as used herein are broad terms, and are to be given their ordinary and customary meaning to a person of ordinary skill in the art (and are not to be limited to a special or customized meaning), and refer without limitation to objects including sensors that are inserted, or configured to be inserted, subcutaneously (i.e. in the layer of fat between the skin and the muscle), intracutaneously (i.e. penetrating the stratum corneum and positioning within the epidermal or dermal strata of the skin), or transcutaneously (i.e. penetrating, entering, or passing through intact skin), which may result in a sensor that has an in vivo portion and an ex vivo portion. The term “indwelling” also encompasses an object which is configured to be inserted subcutaneously, intracutaneously, or transcutaneously, whether or not it has been inserted as such.

The phrase “insertable surface area” as used herein is a broad phrase, and is to be given its ordinary and customary meaning to a person of ordinary skill in the art (and is not to be limited to a special or customized meaning), and refers without limitation to a surface area of an insertable portion of an analyte sensor including, but not limited to, the geometric surface area e.g., planar, flat or substantially planar, and/or coaxial utilized substrates in the analyte sensor as described herein.

The phrase “insertable volume” as used herein is a broad phrase, and is to be given its ordinary and customary meaning to a person of ordinary skill in the art (and is not to be limited to a special or customized meaning), and refers without limitation to a volume ahead of and alongside a path of insertion of an insertable portion of an analyte sensor, as described herein, as well as an incision made in the skin to insert the insertable portion of the analyte sensor. The insertable volume also includes up to 5 mm radially or perpendicular to the volume ahead of and alongside the path of insertion.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In examples of an electrochemical sensor, interfering species are compounds which produce a signal that is not analyte-specific due to a reaction on an electrochemically active surface.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

As used herein, the terms “machine-storage medium,” “device-storage medium,” “computer-storage medium” (referred to collectively as “machine-storage medium” mean the same thing and may be used interchangeably in this disclosure. The terms refer to a single or multiple storage devices and/or media (e.g., a centralized or distributed database, and/or associated caches and servers) that store executable instructions and/or data, as well as cloud-based storage systems or storage networks that include multiple storage apparatus or devices. The terms shall accordingly be taken to include, but not be limited to, solid-state memories, and optical and magnetic media, including memory internal or external to processors. Specific examples of machine-storage media, computer-storage media, and/or device-storage media include non-volatile memory, including by way of example semiconductor memory devices, e.g., erasable programmable read-only memory (EPROM), electrically erasable programmable devices; magnetic disks such as internal hard disks and removable disks; magneto-optical disks; and CD-ROM and DVD-ROM disks. The terms machine-storage media, computer-storage media, and device-storage media specifically exclude carrier waves, modulated data signals, and other such media, at least some of which are covered under the term “signal medium” discussed below.

The term and phrase “mediator” and “redox mediator” as used herein are broad terms and phrases, and are to be given their ordinary and customary meaning to a person of ordinary skill in the art (and is not to be limited to a special or customized meaning), and refers without limitation to any chemical compound or collection of compounds capable of electron transfer, either directly, or indirectly, between an analyte, analyte precursor, analyte surrogate, analyte-reduced or analyte-oxidized enzyme, or cofactor, and an electrode surface held at a potential. In examples the mediator accepts electrons from, or transfer electrons to, one or more enzymes or cofactors, and/or exchanges electrons with the sensor system electrodes. In examples, mediators are transition-metal coordinated organic molecules which are capable of reversible oxidation and reduction reactions. In other examples, mediators may be organic molecules or metals which are capable of reversible oxidation and reduction reactions.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst (e.g., one or more enzymes) for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., glucose or another analyte. In examples, the membrane system comprises an enzyme, which enables an analyte reaction to occur whereby a concentration of the analyte can be measured.

The phrases “machine-readable medium,” ‘computer-readable medium” and ‘device-readable medium” mean the same thing and may be used interchangeably in this disclosure. The phrases are inclusive of both machine-storage media and signal media operably coupled to a sensor, biosensor, analyte sensing device, or analyte monitoring device. Thus, the phrases are inclusive of both storage devices/media and carrier waves/modulated data signals operably coupled to a sensor, biosensor, analyte sensing device, or analyte monitoring device.

The term “micro,” as used herein is a broad term, and is to be given its ordinary and customary meaning to a person of ordinary skill in the art (and is not to be limited to a special or customized meaning), and refers without limitation to a small object or scale of approximately 10⁻⁶ m that is not visible without magnification. The term “micro” is in contrast to the term “macro,” which refers to a large object that may be visible without magnification. Similarly, the term “nano” refers to a small object or scale of approximately 10⁻⁹ m.

The term “noise,” as used herein, is a broad term and is used in its ordinary sense, including, without limitation, a signal detected by the sensor or sensor electronics that is unrelated to analyte concentration and can result in reduced sensor performance. One type of noise has been observed during the few hours (e.g., about 2 to about 24 hours) after sensor insertion. After the first 24 hours, the noise may disappear or diminish, but in some hosts, the noise may last for about three to four days. In some cases, noise can be reduced using predictive modeling, artificial intelligence, and/or algorithmic means. In other cases, noise can be reduced by addressing immune response factors associated with the presence of the implanted sensor, such as using a drug releasing layer with at least one bioactive agent. For example, noise of one or more exemplary biosensors as presently disclosed can be determined and then compared qualitatively or quantitatively. By way of example, by obtaining a raw signal timeseries with a fixed sampling interval (in units of pA), a smoothed version of the raw signal timeseries can be obtained, e.g., by applying a 3rd order lowpass digital Chebyshev Type II filter. Others smoothing algorithms can be used. At each sampling interval, an absolute difference, in units of pA, can be calculated to provide a smoothed timeseries. This smoothed timeseries can be converted into units of mg/dL, (the unit of “noise”), using a glucose sensitivity timeseries, in units of pA/mg/dL, where the glucose sensitivity timeseries is derived by using a mathematical model between the raw signal and reference blood glucose measurements (e.g., obtained from Blood Glucose Meter). Optionally, the timeseries can be aggregated as desired, e.g., by hour or day. Comparison of corresponding timeseries between different exemplary biosensors with the presently disclosed drug releasing layer and one or more bioactive agents provides for qualitative or quantitative determination of improvement of noise.

The term “optional” or “optionally” as used herein is a broad term, and is to be given its ordinary and customary meaning to a person of ordinary skill in the art (and is not to be limited to a special or customized meaning), and, without limitation, means that the subsequently described event or circumstance can or cannot occur, and that the description includes instances where the event or circumstance occurs and instances where it does not.

The term “planar” as used herein is to be interpreted broadly to describe sensor architecture having a substrate including at least a first surface and an opposing second surface, and for example, comprising a plurality of elements arranged on one or more surfaces or edges of the substrate. The plurality of elements can include conductive or insulating layers or elements configured to operate as a circuit. The plurality of elements may or may not be electrically or otherwise coupled. In examples, planar includes one or more edges separating the opposed surfaces.

The term “polyol” is a broad term, and is to be given its ordinary and customary meaning to a person of ordinary skill in the art (and is not to be limited to a special or customized meaning), and refers without limitation to an organic compound containing two or more hydroxyl groups (—OH) and include diols and polyalkylether polyols, where diols include linear or branched akyl diols, for example, 1,2-ethanediol, 1,2-propanediol, 1,3-propanediol, 1,4-butanediol, 1,2-cyclohexanediol, α,ω-telechelic polymer or oligomer with terminal hydroxyl groups, and where polyalkylether polyols include, for example, polyethylene glycol, polypropylene glycol, and poly(tetramethylene ether) glycol. With reference to polyurethane, polyurethane-urea, and polythiourethane-urethane synthesis, the term polyol is used herein synonymously with diols or polyalkylether polyols, unless otherwise indicated, for example, diols can be used as chain extenders together with polyalkylether polyols, or, α,ω-telechelic polymer or oligomer diols can be used together with polyalkylether polyols, or diols can be used as chain extenders together with α,ω-telechelic polymer or oligomer diols together with polyalkylether polyols.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme domain or layer. If the sensor is deemed to be the point of reference and the enzyme domain is positioned nearer to the sensor than the biointerface layer, then the enzyme domain is more proximal to the sensor than the biointerface layer.

The phrase and term “processor module” and “microprocessor” as used herein are each a broad phrase and term, and are to be given their ordinary and customary meaning to a person of ordinary skill in the art (and are not to be limited to a special or customized meaning), and refer without limitation to a computer system, state machine, processor, or the like designed to perform arithmetic or logic operations using logic circuitry that responds to and processes the basic instructions that drive a computer.

The term “semi-continuous” as used herein is a broad term, and is to be given its ordinary and customary meaning to a person of ordinary skill in the art (and is not to be limited to a special or customized meaning), and refers without limitation to a portion, coating, domain, or layer that includes one or more continuous and noncontinuous portions, coatings, domains, or layers. For example, a coating disposed around a sensing region but not about the sensing region is “semi-continuous.”

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In examples, the sensing portion, sensing membrane, and/or sensing mechanism generally comprise an electrode configured to provide signals during electrochemically reactions with one or more membranes covering electrochemically reactive surface. In examples, such sensing portions, sensing membranes, and/or sensing mechanisms are capable of providing specific quantitative, semi-quantitative, qualitative, semi qualitative analytical signals using a biological recognition element combined with a transducing and/or detecting element.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, DNA, RNA, or a protein or aptamer, for example, one or more periplasmic binding protein (PBP) or mutant or fusion protein thereof having one or more analyte binding regions, each region capable of specifically or reversibly binding to and/or reacting with at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, ketone, lactate, potassium, etc., in the biological sample.

In examples, the sensing region or sensing portion can comprise at least a portion of a conductive substrate or at least a portion of a conductive surface, for example, a wire (coaxial) or conductive trace or a substantially planar substrate including substantially planar trace(s), and a membrane. In examples, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme domain, and an electrolyte phase, for example, a free-flowing liquid phase comprising an electrolyte-containing fluid described further below. The terms are broad enough to include the entire device, or only the sensing portion thereof (or something in between).

In examples, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in examples, a sensor has a sensitivity (or slope) of from about 1 to about 100 picoAmps of current for every 1 mg/dL of analyte.

The phrases “signal medium” or “transmission medium” shall be taken to include any form of modulated data signal, carrier wave, and so forth. The phrase “modulated data signal” means a signal that has one or more of its characteristics set or changed in such a matter as to encode information in the signal.

The phrases and terms “small diameter sensor,” “small structured sensor,” and “micro-sensor” as used herein are broad phrases and terms, and are to be given their ordinary and customary meaning to a person of ordinary skill in the art (and are not to be limited to a special or customized meaning), and refer without limitation to sensing mechanisms that are less than about 2 mm in at least one dimension. In further examples, the sensing mechanisms are less than about 1 mm in at least one dimension. In some examples, the sensing mechanism (sensor) is less than about 0.95, 0.9, 0.85, 0.8, 0.75, 0.7, 0.65, 0.6, 0.5, 0.4, 0.3, 0.2, or 0.1 mm. In some examples, the maximum dimension of an independently measured length, width, diameter, thickness, or circumference of the sensing mechanism does not exceed about 2 mm. In some examples, the sensing mechanism is a coaxial sensor, wherein the diameter of the sensor is less than about 1 mm, see, for example, U.S. Pat. No. 6,613,379 to Ward et al. and U.S. Pat. No. 7,497,827 to Brister et al., both of which are incorporated herein by reference in their entirety. In some alternate examples, the sensing mechanism includes electrodes deposited on a planar or substantially planar substrate, wherein the thickness of the implantable portion is less than about 1 mm, see, for example U.S. Pat. No. 6,175,752 to Say et al. and U.S. Pat. No. 5,779,665 to Mastrototaro et. al., both of which are incorporated herein by reference in their entirety.

The phrase “soft segment” as used herein is a broad phrase, and is to be given its ordinary and customary meaning to a person of ordinary skill in the art (and is not to be limited to a special or customized meaning), and refers without limitation to an element of a copolymer, for example, a polyurethane, a polycarbonate-polyurethane, or a polyurethane urea copolymer, which imparts flexibility to the chain. The phrase “soft segment” can be further characterized as an amorphous material with a low Tg, e.g., a Tg not typically higher than ambient temperature or normal mammalian body temperature.

The phrase “solid portions” as used herein is a broad phrase, and is to be given its ordinary and customary meaning to a person of ordinary skill in the art (and is not to be limited to a special or customized meaning), and refers without limitation to portions of a membrane's material having a mechanical structure that demarcates cavities, voids, or other non-solid portions.

The phrases “α,ω-telechelic polymer” or “α,ω-telechelic oligomer” as used herein is a broad phrase, and is to be given its ordinary and customary meaning to a person of ordinary skill in the art (and is not to be limited to a special or customized meaning), and refers without limitation to linear polymers or low-molecular weight oligomers having reactive functional groups on both chain ends or termini, and includes α,ω-homotelechelic polymer/oligomer and α,ω-heterotelechelic polymer/oligomer and mixtures thereof.

The phrases “zwitterionic compounds,” “zwitterionic precursors,” “zwitterionic moieties,” or “zwitterionic derivatives” as used herein are broad phrases, and are to be given its ordinary and customary meaning to a person of ordinary skill in the art (and is not to be limited to a special or customized meaning), and refers without limitation to a betaine compound, precursor, moiety, or derivative thereof. In some examples, the zwitterionic compounds, precursors, moieties, or derivatives thereof comprise a carboxyl, sulfo, or phosphor betaine compound, precursor, or derivative thereof. In some embodiments, the zwitterionic compounds, precursors, or derivatives thereof comprise one or more selected from the group consisting of cocamidopropyl betaine, oleamidopropyl betaine, octyl sulfobetaine, caprylyl sulfobetaine, lauryl sulfobetaine, myristyl sulfobetaine, palmityl sulfobetaine, stearyl sulfobetaine, betaine (trimethylglycine), octyl betaine, phosphatidylcholine, glycine betaine, poly(carboxybetaine) (pCB), poly(sulfobetaine) (pSB), and precursors or derivatives thereof. In some examples, the one or more the zwitterionic compounds, moieties, or derivatives thereof comprise one or more selected from the group consisting of poly(carboxybetaine) (pCB), poly(sulfobetaine) (pSB), and precursors or derivatives thereof. In some examples, the one or more the zwitterionic compounds, moieties, or derivatives thereof comprise one or more vinyl groups for grafting to a α,ω-telechelic polymer or a α,ω-telechelic oligomer.

### Membrane Systems

Membrane systems disclosed herein are suitable for use with implantable devices in contact with a biological fluid. For example, the membrane systems can be utilized with implantable devices, such as devices for monitoring and determining analyte levels in a biological fluid, for example, devices for monitoring glucose levels for individuals having diabetes. In some examples, the analyte-measuring device is a continuous device. The analyte-measuring device can employ any suitable sensing element to provide the raw signal, including but not limited to those involving enzymatic, chemical, physical, electrochemical, spectrophotometric, amperometric, potentiometric, polarimetric, calorimetric, radiometric, immunochemical, or like elements.

Suitable membrane systems for the aforementioned multi-analyte systems and devices can include, for example, membrane systems disclosed in U.S. Pat. Nos. 6,015,572, 5,964,745, and 6,083,523, which are incorporated herein by reference in their entireties for their teachings of membrane systems.

In general, the membrane system includes a plurality of domains, for example, an electrode domain, an interference domain, an enzyme domain, a resistance domain, and a biointerface domain. The membrane system can be deposited on the exposed electroactive surfaces using known thin film techniques (for example, vapor deposition, spraying, electrodepositing, dipping, brush coating, film coating, drop-let coating, and the like). Additional steps may be applied following the membrane material deposition, for example, drying, annealing, and curing (for example, UV curing, thermal curing, moisture curing, radiation curing, and the like) to enhance certain properties such as mechanical properties, signal stability, and selectivity. In a typical process, upon deposition of the resistance domain membrane, a biointerface/drug releasing layer having a “dry film” thickness of from about 0.05 micron (μm), or less, to about 1, 2, 3, 4, 5, 6, 7, 8, 9, 10, 11, 12, 13, 14, 15, or 16 μm is formed. “Dry film” thickness refers to the thickness of a cured film cast from a coating formulation by standard coating techniques.

In certain examples, the biointerface/drug releasing layer is formed of a biointerface polymer, wherein the biointerface polymer comprises one or more membrane domains comprising polyurethane and/or polyurea segments and one or more zwitterionic repeating units. In some examples, the biointerface/drug releasing layer coatings are formed of a polyurethane urea having carboxyl betaine groups incorporated in the polymer and non-ionic hydrophilic polyethylene oxide segments, wherein the polyurethane urea polymer is dissolved in organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the bioprotective polymers are formed of a polyurethane urea having carboxylic acid groups and carboxyl betaine groups incorporated in the polymer and non-ionic hydrophilic polyethylene oxide segments, wherein the polyurethane urea polymer is dissolved in an organic or non-organic solvent system in a coating formulation, and is crosslinked with an a carbodiimide (e.g., 1-ethyl-3-(3-dimethylaminopropyl)carbodiimide (EDC) or polycarbodiimide crosslinkers) and cured at a moderate temperature of about 50° C. In examples, polycarbodiimide crosslinkers are used.

In other examples, the biointerface/drug releasing layer coatings are formed of a polyurethane urea having sulfobetaine groups incorporated in the polymer and non-ionic hydrophilic polyethylene oxide segments, wherein the polyurethane urea polymer is dissolved in an organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the biointerface polymers are formed of a polyurethane urea having unsaturated hydrocarbon groups and sulfobetaine groups incorporated in the polymer and non-ionic hydrophilic polyethylene oxide segments, wherein the polyurethane urea polymer is dissolved in an organic or non-organic solvent system in a coating formulation, and is crosslinked in the presence of initiators with heat or irradiation including UV, LED light, electron beam, and the like, and cured at a moderate temperature of about 50° C. Examples of unsaturated hydrocarbon includes allyl groups, vinyl groups, acrylate, methacrylate, alkenes, alkynes, and the like.

In some examples, tethers are used. A tether is a polymer or chemical moiety which does not participate in the (electro)chemical reactions involved in sensing, but forms chemical bonds with the (electro)chemically active components of the membrane. In some examples these bonds are covalent. In examples, a tether may be formed in solution prior to one or more interlayers of a membrane being formed, where the tether bonds two (electro)chemically active components directly to one another or alternately, the tether(s) bond (electro)chemically active component(s) to polymeric backbone structures. In another example, (electro)chemically active components are comixed along with crosslinker(s) with tunable lengths (and optionally polymers) and the tethering reaction occurs as in situ crosslinking. Tethering may be employed to maintain a predetermined number of degrees of freedom of NAD(P)H for effective enzyme catalysis, where “effective” enzyme catalysis causes the analyte sensor to continuously monitor one or more analytes for a period of from about 5 days to about 15 days or more.

### Membrane Fabrication

Polymers can be processed by solution-based techniques such as spraying, dipping, casting, electrospinning, vapor deposition, spin coating, coating, and the like. Water-based polymer emulsions can be fabricated to form membranes by methods similar to those used for solvent-based materials. In both cases the evaporation of a volatile liquid (e.g., organic solvent or water) leaves behind a film of the polymer. Cross-linking of the deposited film or layer can be performed through the use of multi-functional reactive ingredients by a number of methods. The liquid system can cure by heat, moisture, high-energy radiation, ultraviolet light, or by completing the reaction, which produces the final polymer in a mold or on a substrate to be coated.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membranes and membrane systems, as discussed herein.

Polymers with domains or segments that are functionalized to permit cross-linking can be made by methods at least as discussed herein. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thiol groups). In further examples, polyurethaneurea polymers having aromatic or aliphatic segments having nucleophilic functional groups can be crosslinked with a crosslinking agent that has multiple electrophilic groups. In examples, polycarbodiimide crosslinkers are used. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly(ethylene glycol) diglycidyl ether (PEG-DE), or dicumyl peroxide (DCP). In examples, from about 0.1% to about 15% w/w of cross-linking agent is added relative to the total dry weights of cross-linking agent and polymers added when blending the ingredients. In another example, about 1% to about 10% w/w of cross-linking agent is added relative to the total dry weights of cross-linking agent and polymers added when blending the ingredients. In yet another example, about 5% to about 15% w/w of cross-linking agent is added relative to the total dry weights of cross-linking agent and polymers added when blending the ingredients. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using methods such as spraying, self-assembling monolayers (SAMs), painting, dip-coating, vapor depositing, molding, 3-D printing, slot die coating, pico jet printing, piezo inkjet printing, lithographic techniques (e.g., photolithograph), micro- and nano-pipetting printing techniques, silk-screen printing, etc.). The mixture can then be cured under high temperature (e.g., from about 30° C. to about 150° C.). Other suitable curing methods can include ultraviolet, e-beam, or gamma radiation, for example.

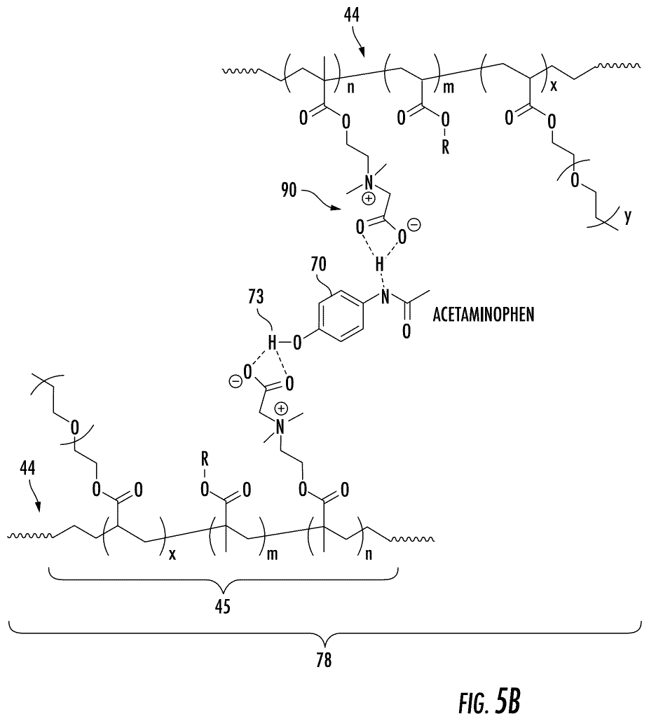

With reference to FIG. 4, an exemplary IL-RL polymer 44 includes a polyurethane or polyurethane urea polymer having hard segment 89 capable of self-association and/or crystallization and soft segment 87 comprising functionalized polymer that provides for both interferent blocking and analyte diffusion resistance.

Example diisocyanates useful as the hard segment component of polyurethane or polyurethane urea polymers of the present disclosure include aliphatic diisocyanates containing from about 4 to about 8 methylene units. Diisocyanates containing cycloaliphatic moieties can also be useful in the preparation of the polymer and copolymer components of the membranes of the present disclosure and are discussed further below. The material that forms the basis of the hydrophobic matrix of the present IL-RL polymer may be selected to exhibit sufficient permeability to allow relevant compounds to pass through it, for example, to allow an oxygen molecule to pass through the membrane from the sample under examination in order to reach the active enzyme or electrochemical electrodes. Other materials can be used to provide the presently disclosed IL-RL polymer, for example, non-polyurethane membranes.

Examples of materials which can be used to make non-polyurethane type membranes include vinyl polymers (including polyvinylimidazole and poly vinylpyridine), polyethers, polyesters, polyamides, inorganic polymers such as polysiloxanes and polycarbosiloxanes, natural polymers such as cellulosic and protein-based materials, and mixtures or combinations thereof. In some examples, these non-polyurethane type membranes include a crosslinking agent in addition to the base polymer, in order to improve mechanical properties and/or tune mass transport of analyte or other species.

### Sensing System

In general, the analyte sensors of the present disclosure include a sensing system with a small structure (e.g., small structured-, micro- or small diameter sensor), for example, a coaxial or planar sensor, in at least a portion thereof. As used herein a “small structure” refers to an architecture with at least one dimension less than about 1 mm. The small-structured sensing mechanism can be coaxial-based, or substrate-based (flat or substantially planar substrate, that can be single or double-sided, which may or may include one or more sensor elements on any of the sides or surfaces), or any other architecture. In some alternate examples, the term “small structure” can also refer to slightly larger structures, such as those having their smallest dimension being greater than about 1 mm, however, the architecture (e.g., mass or size) is designed to minimize the foreign body response due to size and/or mass.

The present disclosure is inclusive of sensor systems including two or more sensors, each sensor being configured to sense a different analyte. The two or more sensors can be configured to function independently or simultaneously to sense two or more analytes concurrently, sequentially, and/or randomly (which is inclusive of events that can take place independently in picoseconds, nanoseconds, milliseconds, seconds, or minutes) or in an alternating or overlapping fashion. The two or more sensors of the sensor system can be communicatively coupled to electronics, e.g., a single transmitter or receiver. The two or more sensors of the sensor system can be communicatively coupled to separate, independent electronics.

In examples of a continuous analyte monitoring system, a first, single, sensor is configured to continuously monitor at least a first analyte (e.g., glycose, glycerol, lactate, bilirubin, oxygen, etc.) and a second, different, analyte (e.g., ketone, LDOPA, etc.). In this example, the single sensor may include a single coaxial or planar sensor configured to monitor the at least first analyte and the second analyte. In another example, a first sensor is configured to monitor the first analyte, and a second sensor is configured to continuously monitor a second analyte (e.g., ketones). Each of the first sensor and the second sensor may be planar, substantially planar, or coaxial, or a combination of two or more top, side, or cross-sectional geometries. In examples, each of the first sensor and the second sensor are communicatively coupled to the same sensor electronics and networking elements to continuously monitor and provide feedback to a device, e.g., a mobile device, tablet, laptop, wearable technology (clothing, jewelry, other accessory) or other loT (internet-of-things) device or combinations of devices. In another example, the first sensor and the second sensor are communicatively coupled to independent sensor electronics and networking elements. Each of the first sensor and the second sensor are positioned in a subject in a subcutaneous layer through a skin layer. In another example, a sensor system is configured as a monolithic sensor body having both the first sensor and the second sensor with their electrodes configured to detect two or more analytes. At least one plurality of electrodes of the sensor system is configured to detect a first analyte, and a second plurality of electrodes is configured to detect a second analyte. The sensor system is positioned in a subject in a subcutaneous layer through a skin layer. In yet another example, a sensor system includes a first sensor and a second sensor, where each sensor of the sensor system includes one or more fiber elements. For example, two or more sensors such as the first sensor and the second sensor may be electrically, mechanically, or otherwise coupled together ex vivo, in vivo, or both. Each of the first sensor and the second sensor of the sensor system is positioned in a subject in a subcutaneous layer through a skin layer.

The analyte sensor device and systems discussed herein may include elements such as on-body wearable devices, wireless communication capabilities, electronics, software, GUI(s), or other elements configured to cause the analyte monitoring systems to continuously monitor analyte levels in a host. Various alerts and actions may be taken in response to this monitoring. As discussed herein, an “on-body” device or wearable device includes devices configured to couple to a host for at least a predetermined period of time via one or more coupling elements including an in-vivo component such as a sensor, and/or adhesives, mechanical elements, electrical elements, magnetic elements, or other combinations of elements. In examples, the sensing system comprises at least one sensing membrane and associated electronics, discussed herein.

### Sensing Membrane

In some examples, a sensing membrane is disposed over the electroactive surfaces of the continuous analyte sensor 100 and includes one or more domains or layers. In general, the sensing membrane functions to control the flux of a biological fluid there through and/or to protect sensitive regions of the sensor from contamination by the biological fluid, for example. Some 1 electrochemical enzyme-based analyte sensors generally include a sensing membrane that controls the flux of the analyte being measured, protects the electrodes from contamination of the biological fluid, and/or provides an enzyme that catalyzes the reaction of the analyte with a co-factor, for example. See, e.g., U.S. Pat. Appl. Pub. No. 2005/0245799 to Brauker et al. and U.S. Pat. No. 7,497,827 to Brister et al., which are incorporated herein by reference in their entirety.

The sensing membranes of the present disclosure can include any membrane configuration suitable for use with any analyte sensor (such as described in more detail herein). In general, the sensing membranes of the present disclosure include one or more domains, all or some of which can be adhered to or deposited on the analyte sensor as is appreciated by a person of ordinary skill in the art. In examples, the sensing membrane generally provides one or more of the following functions: 1) protection of the exposed electrode surface from the biological environment, 2) diffusion resistance (limitation) of the analyte, 3) a catalyst for enabling an enzymatic reaction, 4) limitation or blocking of interfering species, and 5) hydrophilicity at the electrochemically reactive surfaces of the sensor interface, such as described in U.S. Pat. No. 7,497,827 to Brister et al., referenced above. The sensing membranes discussed herein may include one or more adhesive layers positioned in between two adjacent membrane layers. In examples, the one or more adhesive layers can increase robustness and adherence, thus improving the sensing membrane integrity. In various examples, the adhesive layer may include silane groups, polyvinyl alcohol (PVA), glutaraldehyde, or silicone-based or silicone-including materials, or other adhesives or combinations of adhesives.

### Electrode Domain

In some examples, the membrane system comprises an optional electrode domain. The electrode domain is provided to promote and/or enhance an electrochemical reaction between the electroactive surfaces of the working electrode and the reference electrode, and thus the electrode domain is situated more proximal to the electroactive surfaces than the enzyme domain. In some examples, the electrode domain includes a semipermeable coating that maintains a layer of water at the electrochemically reactive surfaces of the sensor, for example, a humectant in a binder material can be employed as an electrode domain; this allows for the full transport of ions in the aqueous environment. The electrode domain can also assist in stabilizing the operation of the sensor by overcoming electrode start-up and drifting problems caused by inadequate electrolyte. The material that forms the electrode domain can also protect against pH-mediated damage that can result from the formation of a large pH gradient due to the electrochemical activity of the electrodes.

In examples, the electrode domain includes a flexible, water-swellable, hydrogel film having a “dry film” thickness of from about 0.05 micron or less to about 20 microns or more. In some examples, the “dry film” thickness is from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns. In further examples, the “dry film” thickness is from about 2, 2.5 or 3 microns to about 3.5, 4, 4.5, or 5 microns. “Dry film” thickness refers to the thickness of a cured film cast from a coating formulation by standard coating techniques.

In certain examples, the electrode domain is formed of a curable mixture of a urethane polymer and a hydrophilic polymer. Coatings are formed of a polyurethane polymer having carboxylate functional groups and non-ionic hydrophilic polyether segments, wherein the polyurethane polymer is crosslinked with a water soluble carbodiimide (e.g., 1-ethyl-3-(3-dimethylaminopropyl)carbodiimide (EDC) or polycarbodiimide crosslinker) in the presence of polyvinylpyrrolidone and cured at a moderate temperature of about 50° C.

In some examples, the electrode domain is deposited by spray or dip-coating the electroactive surfaces of the sensor. In further examples, the electrode domain is formed by dip-coating the electroactive surfaces in an electrode domain solution and curing the domain.

Although an independent electrode domain is described herein, in some examples, sufficient hydrophilicity can be provided in other membranes or their domain and/or enzyme domain (depending on which domain is adjacent to the electroactive surfaces) so as to provide for the full transport of ions in the aqueous environment (e.g. without a distinct electrode domain).

### Optional Non-Mediated Interference Domain

In some examples, an optional interference domain is used in combination with the presently disclosed IL-RL polymer. In some examples, the optional interference domain is provided for non-mediated systems disclosed herein. In some examples, an optional interference domain is provided for non-mediated systems disclosed herein, which generally includes a polymer domain that restricts the flow of one or more interferants to the working electrode. In some examples, the interference domain functions as a molecular sieve that allows analytes and other substances that are to be measured by the electrodes to pass through, while preventing passage of other substances, including interferants such as ascorbate and urea (see U.S. Pat. No. 6,001,067 to Shults). Some known interferants for a glucose-oxidase based electrochemical sensor include acetaminophen, ascorbic acid, bilirubin, cholesterol, creatinine, dopamine, ephedrine, ibuprofen, L-dopa, methyldopa, salicylate, tetracycline, tolazamide, tolbutamide, triglycerides, and uric acid.

Several polymer types that can be utilized as a base material for the interference domain include polyurethanes, polymers having pendant ionic groups (e.g., polyurethane-zwitterion) NAFION™, chitosan, cellulose, or alternating layers of polyallylamine and polyacrylate acid, etc., and polymers having controlled pore size, for example. In examples, the interference domain includes a thin, hydrophobic membrane that is non-swellable and restricts diffusion of low molecular weight species. The interference domain is permeable to relatively low molecular weight substances, such as hydrogen peroxide, but restricts the passage of higher molecular weight substances, including glucose and ascorbic acid. In examples, the interference domain comprises charged species (e.g., polymers with pendent charged groups as disclosed herein) that function to interact with one or more species of the sensing system, such as a cofactor, to reduce or eliminate migration from a domain.

Other systems and methods for reducing or eliminating interference species that can be applied to the membrane system of the present disclosure in combination with the presently disclosed IL-RL polymer are described in U.S. Pat. No. 7,816,004 to Muradov et al., U.S. Pat. Appl. Pub. No. 2005/0176136 to Burd et al., U.S. Pat. No. 7,081,195 to Simpson et al., and U.S. Pat. No. 7,715,893 to Kamath et al. In some alternate examples, a distinct interference domain is not included.

As discussed herein, when two or more sensors are employed in a sensor system, each sensor optionally can include an optional interference domain configured to prevent the same interferent(s) from permeating the membrane in combination with the presently disclosed IL-RL polymer. In another example, when two or more sensors are employed in a sensor system, each sensor optionally includes an optional interference domain configured to prevent the different, or overlapping but also different, interferent(s) from permeating the membrane.

### Mediated System-Optional Interference Domains

Some second generation electrochemical analyte sensor technologies (2^{nd}-gen) leverage immobilized redox mediators to reduce the overpotential required to detect an analyte. This reduction can be significant in contrast to the typical operating potentials for first generation electrochemical analyte sensors (1St-gen, e.g., those operating on the principle of hydrogen peroxide detection on a catalytic metal surface). As an example, 2nd-gen analyte sensor may be biased between +0.0V and +0.3V verses +0.5 to +0.8V for 1st-gen sensors. However, despite this reduction in operating potential and reduction in susceptibility to electroactive interference from endogenous and pharmacologic agents, these 2nd-generation sensors can still succumb to the undue effect of residual interference.

For example, exemplary 2nd-generation analyte sensors utilize polymer-bound covalently-bound redox mediators (e.g., polyvinyl imidazole (PVI)-Os(4,4′-dimethyl-2,2′-bipyridine)2Cl]+/2+) that reduce the overpotential required for the enzymatic detection of a target analyte. An example of such mediator-based sensors includes the systems where undue signal influence arising from the presence of co-circulating endogenous electroactive species can result as evidenced by product labeling warnings regarding large doses of ascorbic acid/ascorbate ion (i.e., Vitamin C), possibly resulting in false hyperglycemia alerts and the like. While charge-selective membranes or further reduction in overpotential may mitigate such interference effects, it may result in a material impact to sensitivity and signal-to-noise figures of merit. Accordingly, at present, mediated electrochemical analyte sensing systems continue to exhibit undue signal influence from endogenous metabolites, such as ascorbic acid.

Thus, the present disclosure includes optional mediated system interference domains developed for 2nd-gen sensor systems in combination with the presently disclosed IL-RL polymer, whether they be continuous glucose monitoring or multianalyte monitoring, e.g., ketone-glucose monitoring, the mediated system interference domains comprising one or more oxidase enzymes, which elicit the enzymatic degradation of an interfering metabolite, or ensemble of metabolites, into a peroxide product, for example hydrogen peroxide. The present disclosure provides domains comprising oxidase enzymes alone or in combination with any of the conventional membranes (electrode, enzyme, resistance domains/layers) used with an indwelling 2nd-generation (e.g., mediated) analyte sensor. Exemplary oxidase enzymes include, for example, ascorbate oxidase or urate oxidase that are configured to catalytically convert an undesired interfering species (e.g., ascorbic acid, uric acid) to a hydrogen peroxide product, which manifests significantly less influence at the bias voltages/overpotentials conventionally applied in 2nd-generation sensing systems. This conversion provides reduced overall concentration of the interfering species at the electrode surface (e.g., trading the flux of the interfering species with the flux of hydrogen peroxide), which provides less detrimental effect to the sensed signal than otherwise would be possible in the presence of the interfering species. The mediated system interference domain can be used alone or in combination with the presently disclosed IL-RL polymer, other interference domains, interference membranes, or interference domains, said other interference domains, interference membranes, or interference domains can comprise the same polymer(s) matrix, for example, without the one or more oxidase enzymes, peroxidase or catalase.

In some examples of the mediated system interference domain, the oxidase enzymes can be combined with one or more peroxidase or peroxidase-like enzymes (e.g., horseradish peroxidase, catalase) to further cleave the generated hydrogen peroxide product from the oxidase enzyme(s), thereby rendering peroxide electroactive agent inert and unable to undergo a redox reaction at the electrode surface. The present disclosure includes deployment of the mediated system in combination with the presently disclosed IL-RL polymer in one or more of the electrode domain, the enzyme domain, the optional resistance domain and the optional interference membrane. The present disclosure includes deployment of the mediated system domain in one or more of the electrode domain, the enzyme domain, and the presently disclosed IL-RL polymer, alone or in combination with an additional interference domain and/or resistance domain.

Thus, in examples, using an exemplary ketone/glucose multianalyte sensor system, can comprise the mediated system interference domain comprising at least one of ascorbate oxidase, urate oxidase, horseradish peroxidase, or catalase is present in an enzyme domain comprising a dehydrogenase enzyme (e.g., beta-hydroxybutyrate dehydrogenase, NADH-acting enzyme (e.g., diaphorase, NAD(P)H dehydrogenase), redox polymer (e.g., PVI-Os(bpy)2Cl), optionally a co-factor (if needed, e.g., NAD+, NADP+) and be optionally crosslinked, e.g., using PEG-DGE, CDI or polycarbodiimide crosslinkers. A presently disclosed IL-RL polymer of a biocompatible material can be applied over the enzyme domain/mediated system.

In another example, using an exemplary ketone/glucose multianalyte sensor system, the mediated system comprising at least one of ascorbate oxidase, urate oxidase, horseradish peroxidase, or catalase is present in the presently disclosed IL-RL polymer, where a separate enzyme domain can be positioned proximal to the electrode and adjacent the mediated system, the enzyme domain comprising dehydrogenase enzyme (e.g., beta-hydroxybutyrate dehydrogenase, NADH-acting enzyme (e.g., diaphorase, NAD(P)H dehydrogenase), redox polymer (e.g., PVI-Os(bpy)2Cl), optionally a co-factor (if needed, e.g., NAD+, NADP+) and be optionally crosslinked, e.g., using PEG-DGE or polycarbodiimide crosslinker.

In another example, using an exemplary ketone/glucose multianalyte sensor system, the mediated system comprising the presently disclosed IL-RL polymer includes at least one of ascorbate oxidase, urate oxidase, horseradish peroxidase, or catalase is present between an enzyme domain and at least one electrode surface. A biocompatible material or drug releasing layer can be applied over the enzyme domain.

In other examples, an exemplary ketone or ketone/glucose multianalyte sensor system that is without a mediator, is provided. In examples, the exemplary ketone or ketone/glucose multianalyte sensor system that is without a metal-based mediator, e.g., osmium complexes of biimidazole and/or imidazole ligands, is provided. In examples, an exemplary ketone or ketone/glucose multianalyte sensor system that is without a metal-based mediator, e.g., osmium complexes of biimidazole and/or imidazole ligands, configured to provide amperometric signal at an applied voltage of greater than +0.2 V, greater than or equal to +0.3 V, greater than or equal to +0.4 V, greater than or equal to +0.5 V, or greater than or equal to +0.6 V, is provided. In examples, an exemplary ketone or ketone/glucose multianalyte sensor system that is without a metal-based mediator and includes an interference layer, is provided. In examples, an exemplary ketone or ketone/glucose multianalyte sensor system that is without a metal-based mediator, e.g., osmium complexes of biimidazole and/or imidazole ligands, configured to provide amperometric signal at an applied voltage of greater than +0.2 V, greater than or equal to +0.3 V, greater than or equal to +0.4 V, greater than or equal to +0.5 V, or greater than or equal to +0.6 V, and includes an interference layer, is provided.

For enzyme-based electrochemical sensors to perform effectively and accurately, the sensor's response is limited by neither enzyme activity nor by co-reactant concentration. Enzymes, including glucose oxidase, can be subject to deactivation as a function of time even in ambient conditions, and this behavior is compensated for in forming the enzyme domain. In some examples, the enzyme domain is constructed of aqueous dispersions of colloidal polyurethane polymers including the enzyme. However, in alternate examples the enzyme domain is constructed from an oxygen-enhancing material, for example, at least one of silicone or fluorocarbon, in order to provide a supply of excess oxygen to ensure that oxygen does not limit the sensing reaction. In some examples, the enzyme is immobilized within the enzyme domain. See U.S. Pat. No. 7,379,765 to Petisce et al.

In examples, the enzyme domain or layer is deposited onto the presently disclosed IL-RL polymer with a “dry film” domain thickness of from about 0.05 micron or less to about 20 microns or more. In other examples, the dry film domain thickness is from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns. In further examples, the dry film domain thickness is from about 2, 2.5 or 3 microns to about 3.5, 4, 4.5, or 5 microns. “Dry film” thickness refers to the thickness of a film cast from a coating formulation by standard coating techniques and includes post-curing of the film.

### Optional Resistance Domain

An immobilized enzyme-based glucose sensor employing oxygen as co-reactant is supplied with oxygen in non-rate-limiting excess in order for the sensor to respond linearly to changes in glucose concentration, while not responding to changes in oxygen concentration. Specifically, when a glucose-monitoring reaction is oxygen limited, linearity is not achieved above minimal concentrations of glucose. Without a semipermeable membrane situated over the enzyme domain to control the flux of glucose and oxygen, a linear response to glucose levels can be obtained only for glucose concentrations of up to about 40 mg/dL. However, in a clinical setting, a linear response to glucose levels is desirable up to at least about 400 mg/dL.

Thus, in examples, the presently disclosed IL-RL polymer, alone in in combination with an optional resistance domain, includes a semi-permeable membrane that controls the flux of oxygen and glucose to the underlying enzyme domain, rendering oxygen in a non-rate-limiting excess. As a result, the upper limit of linearity of glucose measurement is extended to a much higher value than that which is achieved without a resistance domain. In examples, the presently disclosed IL-RL polymer alone or in combination with the optional resistance domain exhibits an oxygen to glucose permeability ratio of from about 50:1 or less to about 400:1 or more. In further examples, the oxygen to glucose permeability ratio is about 200:1.

In examples, the membrane system includes an optional resistance domain in combination with the presently disclosed IL-RL polymer, the optional resistance domain being disposed more distal from the electroactive surfaces than the enzyme domain and/or the presently disclosed IL-RL polymer. Although the following description is directed to an optional resistance domain for a glucose sensor, the resistance domain can be modified for facilitating detection of the concentrations(s) of other analytes and co-reactants as well. In examples, the optional resistance domain in combination with the presently disclosed IL-RL polymer is configured to control the flux of oxygen through the membrane. In another example, the optional resistance domain in combination with the presently disclosed IL-RL polymer is configured to control the flux of an analyte or co-reactant other than oxygen through the membrane. In yet another example, the optional resistance domain in combination with the presently disclosed IL-RL polymer is configured to control the flux of two or more different analytes through the membrane.

In alternate examples, a lower ratio of oxygen-to-glucose can be sufficient to provide excess oxygen by using a high oxygen solubility domain (for example, a silicone or fluorocarbon-based material or domain) to enhance the supply/transport of oxygen to the transducing element domain. If more oxygen is supplied to the enzyme, then more glucose can also be supplied to the transducing element without creating an oxygen rate-limiting excess. In alternate examples, the resistance domain is formed from a silicone composition, such as is described in U.S. Pat. Appl. Pub. No. 2005/0090607 to Tapsak et al.

In examples, the optional resistance domain includes a polyurethane membrane with both hydrophilic and hydrophobic regions similar to the presently disclosed IL-RL polymer. The hydrophilic and hydrophobic regions may be used in combination to control the diffusion of an analyte or analytes (e.g., glucose, oxygen, ketones, lactate, uric acid, etc.) or substrates to an analyte sensor. A suitable optional resistance domain comprises a hydrophobic polymer component of a polyurethane, or polyurethane urea.

The individual components or layers of the sensing system can be deposited, starting with the electroactive surfaces of the electrode material and subsequently depositing other components, using known thin or thick film techniques (for example, spraying, electro-depositing, dipping, or the like). It is noted that the sensing system that surrounds the working electrode does not have to be the same structure as the sensing system that surrounds a reference electrode, etc. For example, the transducing element domain deposited over the working electrode does not necessarily need to be deposited over the reference and/or counter electrodes.

In examples, the sensor is an enzyme-based electrochemical sensor, wherein the working electrode measures the hydrogen peroxide produced by the enzyme catalyzed reaction of analyte (e.g., glucose) being detected and creates a measurable electronic current (for example, detection of glucose utilizing glucose oxidase produces hydrogen peroxide as a by-product, H₂O₂ reacts with the surface of the working electrode producing two protons (2H⁺), two electrons (2e⁻) and one molecule of oxygen (O₂) which produces the electronic current being detected), such as described in more detail above and as is appreciated by a person of ordinary skill in the art. In some examples, one or more potentiostats are employed to monitor the electrochemical reaction at the electroactive surface of the working electrode(s). The potentiostat applies a constant potential to the working electrode and its associated reference electrode to determine the current produced at the working electrode. The current that is produced at the working electrode (and flows through the circuitry to the counter electrode) is substantially proportional to the amount of H₂O₂ that diffuses to the working electrode. The output signal is typically a raw data stream, e.g., a raw signal processed by algorithms prior to display of values, that is used to provide a useful value of the measured analyte concentration in a host to the host or doctor, for example.

Some alternate analyte sensors that can benefit from the systems and methods of the present disclosure include U.S. Pat. No. 5,711,861 to Ward et al., U.S. Pat. No. 6,642,15 to Vachon et al., U.S. Pat. No. 6,654,625 to Say et al., U.S. Pat. No. 6,565,509 to Say et al., U.S. Pat. No. 6,514,718 to Heller, U.S. Pat. No. 6,465,66 to Essenpreis et al., U.S. Pat. No. 6,214,185 to Offenbacher et al., U.S. Pat. No. 5,310,469 to Cunningham et al., and U.S. Pat. No. 5,683,562 to Shaffer et al., U.S. Pat. No. 6,579,690 to Bonnecaze et al., U.S. Pat. No. 6,484,46 to Say et al., U.S. Pat. No. 6,512,939 to Colvin et al., U.S. Pat. No. 6,424,847 to Mastrototaro et al., U.S. Pat. No. 6,424,847 to Mastrototaro et al., for example. Each of the above patents are incorporated in their entirety herein by reference and are not inclusive of all applicable analyte sensors; in general, it should be understood that the disclosed examples are applicable to a variety of analyte sensor configurations. in other examples of sensor systems including biointerface/drug release layer(s), the sensor may be a planar or substantially planar sensor.

In examples, the transducing element comprises one or more membranes that can comprise one or more layers, membranes and/or domains, each of the one or more layers, membranes or domains can independently comprise one or more signal transducers, e.g., enzymes, RNA, DNA, aptamers, binding proteins, etc. As used herein, transducing elements includes enzymes, ionophores, RNA, DNA, aptamers, and binding proteins.

In examples, the transducing element is present in one or more membranes, layers, or domains formed over a sensing region. In examples, such sensors can be configured using one or more enzyme domains, e.g., membrane domains including enzyme domains, also referred to as EZ layers (“EZLs”), each enzyme domain may comprise one or more enzymes. Reference hereinafter to an “enzyme layer” is intended to include all or part of an enzyme domain, either of which can be all or part of a membrane system as discussed herein, for example, as a single layer, as two or more layers, as pairs of bi-layers, or as combinations thereof.

### NAD Based Analyte Sensor Platform

Nicotinamide adenine dinucleotide (NAD(P)⁺/NAD(P)H) is a coenzyme, e.g., a dinucleotide that consists of two nucleotides joined through their phosphate groups. One nucleotide contains an adenine nucleobase and the other nicotinamide. NAD exists in two forms, e.g., an oxidized form (NAD(P)+) and reduced form (NAD(P)H) (H=hydrogen). The reaction of NAD+ and NADH is reversible, thus, the coenzyme can continuously cycle between the NAD(P)+/and NAD(P)H forms essentially without being consumed.

In examples, one or more enzyme domains of the sensing system of the presently disclosed continuous analyte sensor device comprise an amount of NAD+ or NADH for providing transduction of a detectable signal corresponding to the presence or concentration of one or more analytes in combination with the presently disclosed IL-RL polymer. In examples, one or more enzyme domains of the sensing system comprising the presently disclosed IL-RL polymer of the continuous analyte sensor device comprises an excess amount of NAD+ or NADH for providing extended transduction of a detectable signal corresponding to the presence or concentration of one or more analytes. In examples, the presently disclosed IL-RL polymer inhibits or prevents migration of NAD+ or NADH from the sensing system.

In examples, NAD, NADH, NAD+, NAD(P)+, ATP, flavin adenine dinucleotide (FAD), magnesium (Mg++), pyrroloquinoline quinone (PQQ), and functionalized derivatives thereof can be used in combination with one or more enzymes in the continuous analyte sensor device with the sensing system comprising the IL-RL polymer. In examples, NAD, NADH, NAD+, NAD(P)+, ATP, flavin adenine dinucleotide (FAD), magnesium (Mg++), pyrroloquinoline quinone (PQQ), and functionalized derivatives are incorporated in the sensing system. In examples, NAD, NADH, NAD+, NAD(P)+, ATP, flavin adenine dinucleotide (FAD), magnesium (Mg++), pyrroloquinoline quinone (PQQ), and functionalized derivatives are dispersed or distributed in one or more membranes or domains of the sensing system with the sensing system comprising the IL-RL polymer.

In aspects of the present disclosure, continuous sensing of one or more or two or more analytes using NAD+ dependent enzymes is provided in one or more membranes or domains of the sensing system with the sensing system comprising the IL-RL polymer. In examples, the membrane or domain provides retention and stable recycling of NAD+ as well as mechanisms for transducing NADH oxidation or NAD+ reduction into measurable current with amperometry. In examples, described below, continuous, sensing of analytes, either reversibly bound or at least one of which are oxidized or reduced by NAD+ dependent enzymes, for example, ketones (beta-hydroxybutyrate dehydrogenase), glycerol (glycerol dehydrogenase), cortisol (11β-hydroxysteroid dehydrogenase), glucose (glucose dehydrogenase), alcohol (alcohol dehydrogenase), aldehydes (aldehyde dehydrogenase), and lactate (lactate dehydrogenase) is provided with the sensing system comprising the IL-RL polymer. In other examples, described below, membranes are provided that enable the continuous, on-body sensing of multiple analytes which utilize FAD-dependent dehydrogenases, such as fatty acids (Acyl-CoA dehydrogenase) with the sensing system comprising the IL-RL polymer containing.

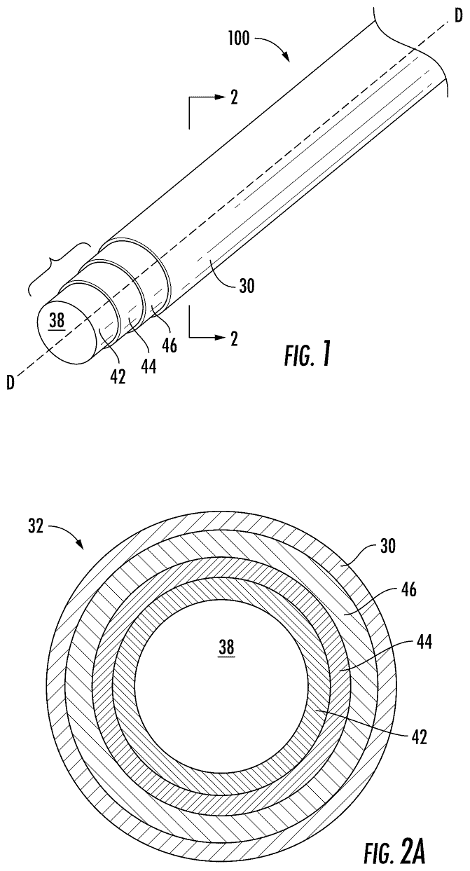

FIG. 1 is a depiction of an exemplary sensor 100 having a plurality of layers, domains, or membranes 42, 44, 46 and reference electrode 30 about a working electrode 38. FIG. 1 is a perspective sectional view of an exemplary example of a continuous analyte sensor 34, also referred to as an analyte sensor, illustrating the sensing system. In some examples, the sensing system is adapted for insertion under the host's skin, and the remaining body of the sensor (e.g. electronics, etc.) can reside ex vivo. In the illustrated example, the analyte sensor 34 includes two electrodes, i.e., a working electrode 38 and at least one additional electrode 30, which may function as a counter or reference electrode, hereinafter referred to as the reference electrode 30.

In some examples, an enzyme domain 42, also referred to as the enzyme layer, may be used and is situated less distal from the electrochemically reactive surfaces than the IL-RL polymer domain 44. The enzyme domain comprises a catalyst configured to react with an analyte. In examples, the enzyme domain is an immobilized enzyme domain 42 including glucose oxidase. In other examples, the enzyme domain 42 can be impregnated with other oxidases, for example, galactose oxidase, cholesterol oxidase, amino acid oxidase, alcohol oxidase, lactate oxidase, or uricase. For example, for an enzyme-based electrochemical glucose sensor to perform well, the sensor's response should neither be limited by enzyme activity nor cofactor concentration.

In some examples, the catalyst (enzyme) can be impregnated or otherwise immobilized into the bioprotective or diffusion resistance domain such that a separate enzyme domain 42 is not required (e.g. wherein a unitary domain is provided including the functionality of the bioprotective domain, diffusion resistance domain, and enzyme domain). In some examples, the enzyme domain 42 is formed from a polyurethane, for example, aqueous dispersions of colloidal polyurethane polymers including the enzyme.

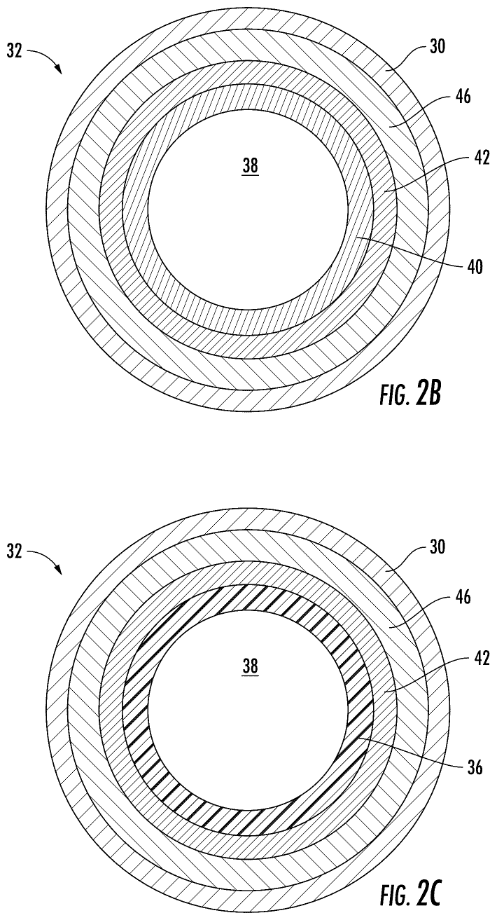

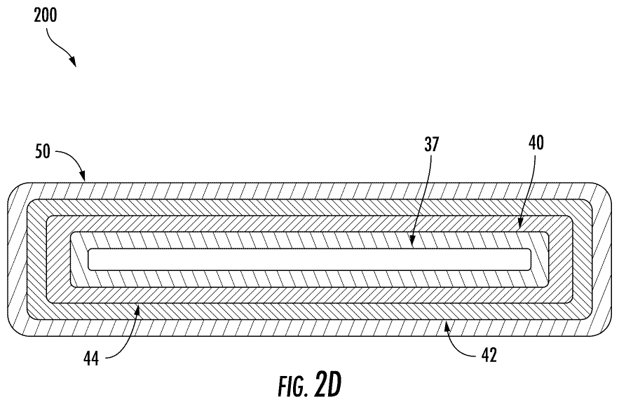

It is contemplated that in some examples, such as the example illustrated in FIG. 2B, an optional interference domain 40, also referred to as the interference layer, is used in combination with or in addition to the presently disclosed IL-RL polymer. It is contemplated that in some examples, such as the example illustrated in FIG. 2B, an optional interference domain 40, also referred to as the interference layer, is used in combination with or in addition to the presently disclosed IL-RL polymer, bioprotective domain and the enzyme domain. The optional interference domain 40 may complement the presently disclosed IL-RL polymer and contribute to substantially reducing the permeation of one or more interferents into the electrochemically reactive surfaces. In examples, similar to the presently disclosed IL-RL polymer, the optional interference domain 40 is configured to be much less permeable to one or more of the interferents than to the measured species. It is also contemplated that in some examples, where interferent blocking may be provided by the presently disclosed IL-RL polymer, alone or in combination with the bioprotective domain (e.g. via a surface-active group-containing polymer of the bioprotective domain), a separate interference domain is excluded.

The present disclosure provides an improvement to analyte monitoring, including continuous analyte monitoring by providing for substitution of a discrete interference layer (IL) functioning to screen out any interferent molecules, and a discrete resistance layer (RL) membrane functioning as an analyte modulator with a polymer with both interference blocking and analyte modulation functionality. The present disclosure thus provides an improvement to current analyte monitoring systems at least by reducing or eliminating the need to layer discrete IL and RL polymer membranes, layers, or domains, thus reducing or eliminating manufacturing steps, and improving product performance, which provides a technical solution to the aforementioned technical problem.

Thus, the present disclosure provides for polymer layers, membranes, or domains with dual functionality: analyte diffusion resistance; and interferent blocking functionality. The present disclosure thus provides for a “IL-RL polymer” that can be configured as a membrane, layer, or domain with dual functionality for amperometric continuous analyte monitors or devices. Thus, the present disclosure provides for polymer layers, membranes, or domains with dual functionality for amperometric continuous analyte monitors, for example, continuous glucose monitors (CGM's), continuous ketone monitors, and other amperometric continuous (multi-)analyte monitors.

In examples, the present disclosure provides for grafted α,ω-telechelic or α,ω-homotelechelic polymer or oligomer intermediates suitable for thermoplastic polyurethane or polyurethane urea polymer synthesis (hereafter collectively referred to as “TPU or TPUU”). The grafted α,ω-telechelic or α,ω-homotelechelic polymer or oligomer intermediates provide for sufficient reactivity for polymerization with diisocyanates as well as sufficient analyte diffusion resistance and interferent blocking functionality.

In examples, the α,ω-telechelic or α,ω-homotelechelic polymer or oligomer intermediates are polyols, dithiols, dihalides or combinations thereof, are grafted with monomers, oligomers, and/or polymers with functional groups capable of interacting with known interferents. In examples, grafted α,ω-telechelic or α,ω-homotelechelic polymer or oligomer intermediates are used to prepare TPU or TPUU polymers suitable for continuous analyte monitoring systems. In examples, the presently disclosed TPU or TPUU polymers with grafted α,ω-telechelic or α,ω-homotelechelic polymer or oligomer are incorporated within the backbone structure of a primary TPU component polymer membrane.

In another example, the presently disclosed TPU or TPUU polymers with grafted α,ω-telechelic or α,ω-homotelechelic polymer or oligomer are incorporated within the backbone structure of a primary TPU component so as to provide for the manufacture of continuous analyte monitoring systems with the elimination of a discrete interferent layer (IL) and discrete diffusion resistance layer (RL), which is advantageous as eliminating variation in the manufacturing process, thus improving quality control during manufacturing.

FIG. 2A is a cross-sectional view through a sensor 10 of FIG. 1 on line 2-2, illustrating one example of a membrane system 32 of the plurality of layers, domains, or membranes 42, 44, 46. In this particular example, the “membrane system” 32 includes an enzyme domain 42, the presently disclosed IL-RL polymer domain 44, and a bioprotective domain 46 located around the working electrode 38, all of which are described in more detail elsewhere herein.

In some examples, the membrane system may include a bioprotective domain 46, also referred to as a cell-impermeable domain or biointerface domain, comprising a surface-modified base polymer as described in more detail elsewhere herein. However, the sensing membranes 32 of some examples can also include a plurality of domains or layers including, for example, an electrode domain (e.g., as illustrated in the FIG. 2C), an interference domain (e.g., as illustrated in FIG. 2B), or a cell disruptive domain (not shown), such as described in more detail elsewhere herein and in U.S. Patent Publication No. US-2006-0036145-A1, which is incorporated herein by reference in its entirety.

It is contemplated that in some examples, such as the example illustrated in FIG. 2C, an optional electrode domain 36, also referred to as the electrode layer, may be provided, in addition to the IL-RL polymer, bioprotective domain and the enzyme domain; however, in other examples, the functionality of the electrode domain may be incorporated into the bioprotective domain so as to provide a unitary domain that includes the functionality of the bioprotective domain, presently disclosed IL-RL polymer (as interference blocking and diffusion limiting), enzyme domain, and electrode domain.

Although the exemplary examples illustrated in FIGS. 2A-2C involve circumferentially extending membrane systems, the membranes described herein may be applied to any planar or non-planar surface, for example, the substrate-based sensor structure of U.S. Pat. No. 6,565,509 to Say et al. Thus, with reference to FIG. 2D a planar version of sensor 200 can include a sensing membrane with multiple layers or domains. For example, the planar version can include conductive surface or trace 37, optional interference domain 40, enzyme domain 42, and IL-RL polymer 44, in addition to other variations of domains, such as a drug releasing membrane 50 as discussed elsewhere herein.

In some examples, the IL-IR polymer 44 is used in combination with, or is included in another domain or layer of the membrane system (e.g., where the functionality of both domains is incorporated into one domain, i.e., the IL-RL polymer 44 or the bioprotective domain). However, it is understood that the membrane system 32 can be modified for use in other devices, for example, by including only one or more of the domains, or additional domains.

It is to be understood that membrane system 32 can be modified for other sensors, for example, so as to include fewer or additional layers. For example, in some examples, the membrane system may comprise one electrode layer, one enzyme layer, and two bioprotective layers, but in other examples, the membrane system may comprise one electrode layer, two enzyme layers, and one bioprotective layer. In some examples, the bioprotective layer may be configured to function as a diffusion resistance domain and control the flux of the analyte (e.g., glucose) to the underlying membrane layers. In some examples, IL-RL polymer 44 may be configured to function as a diffusion resistance domain and control the flux of the analyte to the underlying membrane layers.

In some examples, the sensing membrane can be deposited on the electroactive surfaces of the electrode material using known thin or thick film techniques (for example, spraying, electro-depositing, dipping, or the like). It should be appreciated that the sensing membrane located over the working electrode does not have to have the same structure as the sensing membrane located over the reference electrode; for example, the enzyme domain deposited over the working electrode does not necessarily need to be deposited over the reference or counter electrodes.

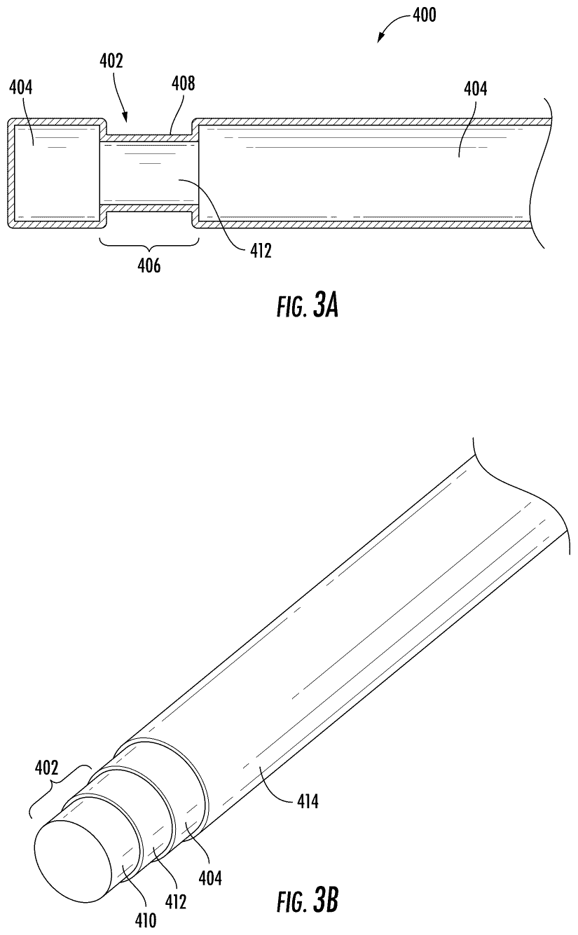

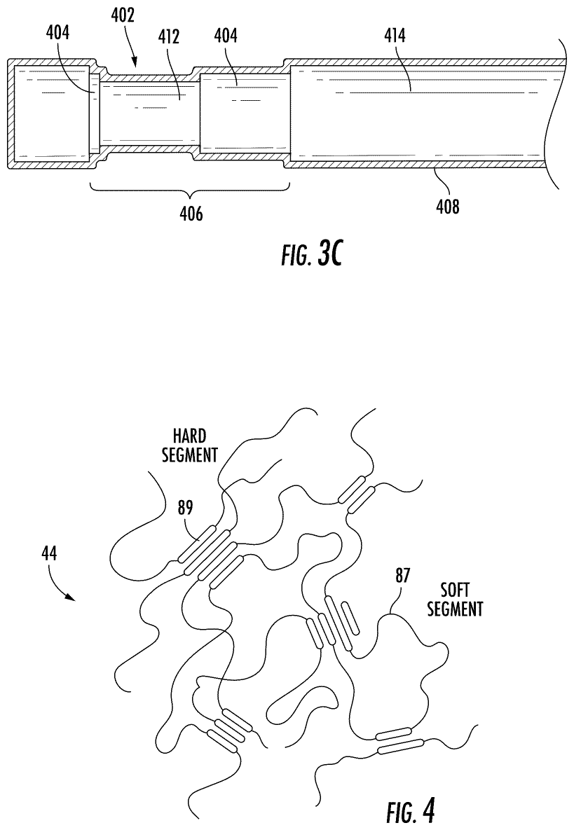

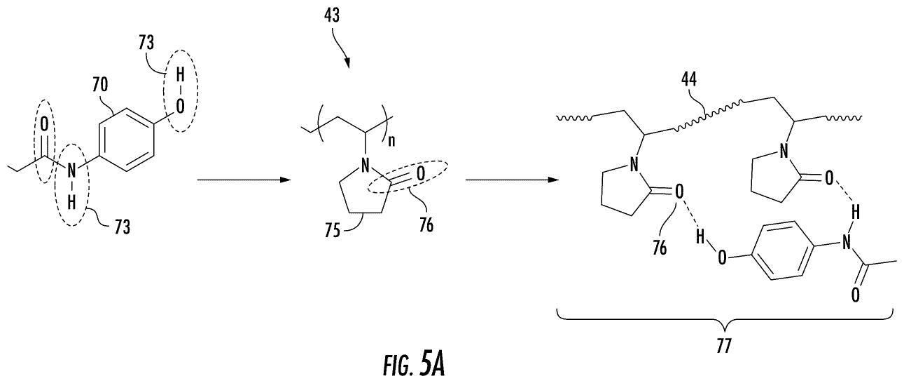

FIGS. 3A through 3C illustrate an alternate embodiment of the in vivo portion of a continuous analyte sensor 400, which includes an elongated conductive body 402. The elongated conductive body 402 includes a core 410 (see FIG. 3B) and a first layer 412 at least partially surrounding the core. The first layer includes a working electrode (for example, located in window 406) and a membrane 408 located over the working electrode. In some embodiments, the core and first layer can be of a single material (such as, for example, platinum). In some embodiments, the elongated conductive body is a composite of at least two materials, such as a composite of two conductive materials, or a composite of at least one conductive material and at least one non-conductive material. In some embodiments, the elongated conductive body comprises a plurality of layers. In certain embodiments, there are at least two concentric or annular layers, such as a core formed of a first material and a first layer formed of a second material. However, additional layers can be included in some embodiments. In some embodiments, the layers are coaxial.

The elongated conductive body may be long and thin, yet flexible and strong. For example, in some embodiments, the smallest dimension of the elongated conductive body is less than about 0.1 inches, 0.075 inches, 0.05 inches, 0.025 inches, 0.01 inches, 0.004 inches, or 0.002 inches. While the elongated conductive body is illustrated in FIGS. 3A through 3C as having a circular cross-section, in other embodiments the cross-section of the elongated conductive body can be ovoid, rectangular, triangular, polyhedral, star-shaped, C-shaped, T-shaped, X-shaped, Y-Shaped, irregular, or the like. In one embodiment, a conductive wire electrode is employed as a core. To such a clad electrode, two additional conducting layers may be added (e.g., with intervening insulating layers provided for electrical isolation). The conductive layers can be comprised of any suitable material. In certain embodiments, it can be desirable to employ a conductive layer comprising conductive particles (i.e., particles of a conductive material) in a polymer or other binder.

The materials used to form the elongated conductive body (such as, for example, stainless steel, titanium, tantalum, platinum, platinum-iridium, iridium, certain polymers, and/or the like) can be strong and hard, and therefore are resistant to breakage. In some embodiments, the sensor's small diameter provides flexibility to these materials, and therefore to the sensor as a whole. Thus, the sensor can withstand repeated forces applied to it by surrounding tissue.

In addition to providing structural support, resiliency and flexibility, in some embodiments, the core 410, or a component thereof, provides electrical conduction for an electrical signal from the working electrode to sensor electronics (not shown). In some embodiments, the core 410 comprises a conductive material, such as stainless steel, titanium, tantalum, a conductive polymer, and/or the like. However, in other embodiments, the core is formed from a non-conductive material, such as a non-conductive polymer. In yet other embodiments, the core comprises a plurality of layers of materials. For example, in one embodiment the core includes an inner core and an outer core. In a further embodiment, the inner core is formed of a first conductive material and the outer core is formed of a second conductive material. For example, in some embodiments, the first conductive material is stainless steel, titanium, tantalum, a conductive polymer, an alloy, and/or the like, and the second conductive material is a conductive material selected to provide electrical conduction between the core and the first layer, and/or to attach the first layer to the core (that is, if the first layer is formed of a material that does not attach well to the core material). In another embodiment, the core is formed of a non-conductive material (such as, for example, a non-conductive metal and/or a non-conductive polymer) and the first layer is formed of a conductive material, such as stainless steel, titanium, tantalum, a conductive polymer, and/or the like. The core and the first layer can be of a single (or same) material, such as platinum. One skilled in the art appreciates that additional configurations are possible.

Referring again to FIGS. 3A-3C, the first layer 412 can be formed of a conductive material and the working electrode can be an exposed portion of the surface of the first layer 412. Accordingly, the first layer 412 can be formed of a material configured to provide a suitable electroactive surface for the working electrode, a material such as, but not limited to, platinum, platinum-iridium, gold, palladium, iridium, graphite, carbon, a conductive polymer, an alloy and/or the like.

As illustrated in FIG. 3B-3C, a second layer 404 surrounds at least a portion of the first layer 412, thereby defining the boundaries of the working electrode. In some embodiments, the second layer 404 serves as an insulator and is formed of an insulating material, such as polyimide, polyurethane, parylene, or any other known insulating materials. For example, in one embodiment the second layer is disposed on the first layer and configured such that the working electrode is exposed via window 406. In some embodiments, an elongated conductive body, including the core, the first layer and the second layer, is provided. A portion of the second layer can be removed to form a window 406, through which the electroactive surface of the working electrode (that is, the exposed surface of the first layer 412) is exposed. In some embodiments, a portion of the second and (optionally) third layers can be removed to form the window 406, thus exposing the working electrode. Removal of coating materials from one or more layers of the elongated conductive body (for example, to expose the electroactive surface of the working electrode) can be performed by hand, excimer lasing, chemical etching, laser ablation, grit-blasting, or the like.

The sensor can further comprise a third layer 414 comprising a conductive material. For example, the third layer 414 may comprise a reference electrode, which may be formed of a silver-containing material that is applied onto the second layer 404 (that is, the insulator).

The elongated conductive body 402 can further comprise one or more intermediate layers (not shown) located between the core 410 and the first layer 412. For example, the intermediate layer can be one or more of an insulator, a conductor, a polymer, and/or an adhesive.

Referring again to FIGS. 3B-3C, the reference electrode 414 can comprise a silver-containing material (e.g., silver/silver chloride) applied over at least a portion of the insulating material 404, as discussed in greater detail elsewhere herein. For example, the silver-containing material can be applied using thin film and/or thick film techniques, such as but not limited to dipping, spraying, printing, electro-depositing, vapor deposition, spin coating, and sputter deposition, as described elsewhere herein. For example, a silver or silver chloride-containing paint (or similar formulation) can be applied to a reel of the insulated conductive core. Alternatively, the reel of insulated elongated body (or core) is cut into single unit pieces (that is, “singularized”), and silver-containing ink is pad printed thereon. In still other examples, the silver-containing material is applied as a silver foil. For example, an adhesive can be applied to an insulated elongated body, around which the silver foil can then be wrapped in. Alternatively, the sensor can be rolled in Ag/AgCl particles, such that a sufficient amount of silver sticks to and/or embeds into and/or otherwise adheres to the adhesive for the particles to function as the reference electrode. In some examples, the sensor's reference electrode includes a sufficient amount of chloridized silver that the sensor measures and/or detects the analyte for at least three days.

FIG. 3B is a schematic illustrating an example of an elongated conductive body 402, or elongated body, wherein the elongated conductive body is formed from at least two materials and/or layers of conductive material, as described in greater detail elsewhere herein. The term “electrode” can be used herein to refer to the elongated conductive body, which includes the electroactive surface that detects the analyte. In some examples, the elongated conductive body provides an electrical connection between the electroactive surface (that is, the working electrode) and the sensor electronics (not shown). In certain examples, each electrode (that is, the elongated conductive body on which the electroactive surface is located) is formed from a fine wire with a diameter of from about 0.001 inches or less to about 0.01 inches or more. Each electrode can be formed from, for example, a plated insulator, a plated wire, or bulk electrically conductive material. For example, in some examples, the wire and/or elongated conductive body used to form a working electrode is about 0.002, 0.003, 0.004, 0.005, 0.006, 0.007, 0.008, 0.009, 0.01, 0.015, 0.02, 0.025, 0.03, 0.035, 0.04 or 0.045 inches in diameter.

Furthermore, the first layer can comprise an electroactive surface (that is, the portion exposed through the window 406). The exposed electroactive surface can be the working electrode. For example, if the sensor is an enzymatic electrochemical analyte sensor, the analyte enzymatically reacts with an enzyme in the membrane covering at least a portion of the electroactive surface. The reaction can generate electrons (e⁻) that are detected at the electroactive surface as a measurable electronic current. For example, in the detection of glucose wherein glucose oxidase produces hydrogen peroxide as a byproduct, hydrogen peroxide reacts with the surface of the working electrode producing two protons (2H⁺), two electrons (2e⁻) and one molecule of oxygen (O₂), which produces the electronic current being detected.

As illustrated in FIGS. 3A and 3C, the sensor can also include a membrane 408, such as those discussed elsewhere herein, for example, with reference to FIGS. 2A-2C. The membrane 408 can include an enzyme layer (not shown), as described elsewhere herein. For example, the enzyme layer can include a catalyst or enzyme configured to react with an analyte. For example, the enzyme layer can be an immobilized enzyme layer including an oxidase. In other examples, the enzyme layer can be impregnated with and/or combined with other oxidases, including, for example, galactose oxidase, cholesterol oxidase, amino acid oxidase, alcohol oxidase, lactate oxidase, 3-hydroxybutyrate dehydrogenase (3HBDH), uricase or combinations thereof.

As previously described with reference to FIG. 3A and as illustrated in FIG. 3C, an insulator 404 is disposed on at least a portion of the elongated conductive body 402. In some examples, the sensor is configured and arranged such that the elongated body includes a core 410 and a first layer 412, and a portion of the first layer 412 is exposed via window 406 in the insulator 404. In other examples, the sensor is configured and arranged such that the elongated body 402 includes a core 410 embedded in an insulator 404, and a portion of the core 410 is exposed via the window 406 in the insulator 404. For example, the insulating material can be applied to the elongated body 402 (by, for example, screen-, ink-jet and/or block-print) in a configuration designed to leave at least a portion of the first layer's 412 surface (or the core's 410 surface) exposed. For example, the insulating material can be printed in a pattern that does not cover a portion of the elongated body 402. Alternatively, a portion of the elongated body 402 can be masked prior to application of the insulating material. Removal of the mask, after insulating material application, can expose the portion of the elongated body 402.

In some examples, the insulating material 404 comprises a polymer, for example, a non-conductive (that is, dielectric) polymer. Dip-coating, spray-coating, vapor-deposition, printing and/or other thin film and/or thick film coating or deposition techniques can be used to deposit the insulating material on the elongated body 402 and/or core 410. For example, in some examples, the insulating material is applied as a layer of from about less than 5 microns, or from 5, 10 or 15-microns to about 20, 25, 30 or 35-microns or more in thickness. The insulator can be applied as a single layer of material, or as two or more layers, which are comprised of either the same or different materials, as described elsewhere herein. Alternatively, the conductive core may not require a coating of insulator. In some examples, the insulating material defines an electroactive surface of the analyte sensor (that is, the working electrode). For example, a surface of the conductive core (such as, for example, a portion of the first layer 412) can either remain exposed during the insulator application, or a portion of applied insulator can be removed to expose a portion of the conductive core's surface, as described above.

In some examples, in which the sensor has an insulated elongated body or an insulator disposed upon a conductive structure, a portion of the insulating material can be stripped or otherwise removed, for example, by hand, excimer lasing, chemical etching, laser ablation, grit-blasting (such as, for example, with sodium bicarbonate or other suitable grit), or the like, to expose the electroactive surfaces. In one exemplary example, grit blasting is implemented to expose the electroactive surface(s), for example, by utilizing a grit material that is sufficiently hard to ablate the polymer material yet also sufficiently soft so as to minimize or avoid damage to the underlying metal electrode (for example, a platinum electrode). Although a variety of “grit” materials can be used (such as, for example, sand, talc, walnut shell, ground plastic, sea salt, and the like), in some examples, sodium bicarbonate is an advantageous grit-material because it is sufficiently hard to ablate, e.g., a parylene coating without damaging, e.g., an underlying platinum conductor. An additional advantage of sodium bicarbonate blasting includes its polishing action on the metal as it strips the polymer layer, thereby eliminating a cleaning step that might otherwise be necessary. Alternatively, a portion of an electrode or other conductive body can be masked prior to depositing the insulator in order to maintain an exposed electroactive surface area.

The electroactive surface of the working electrode can be exposed by formation of a window 406 in the insulator 404. The electroactive window 406 of the working electrode can be configured to measure the concentration of an analyte.

In some examples, a silver wire is formed onto and/or fabricated into the sensor and subsequently chloridized to form a silver/silver chloride reference electrode. Advantageously, chloridizing the silver wire as described herein enables the manufacture of a reference electrode with good in vivo performance. By controlling the quantity and amount of chloridization of the silver to form silver/silver chloride, improved break-in time, stability of the reference electrode and extended life can be obtained in some examples. Additionally, use of silver chloride as described above allows for relatively inexpensive and simple manufacture of the reference electrode.

FIG. 4 is a representation of a polymer with hard segments and soft segments in accordance with various technologies described in the present disclosure.

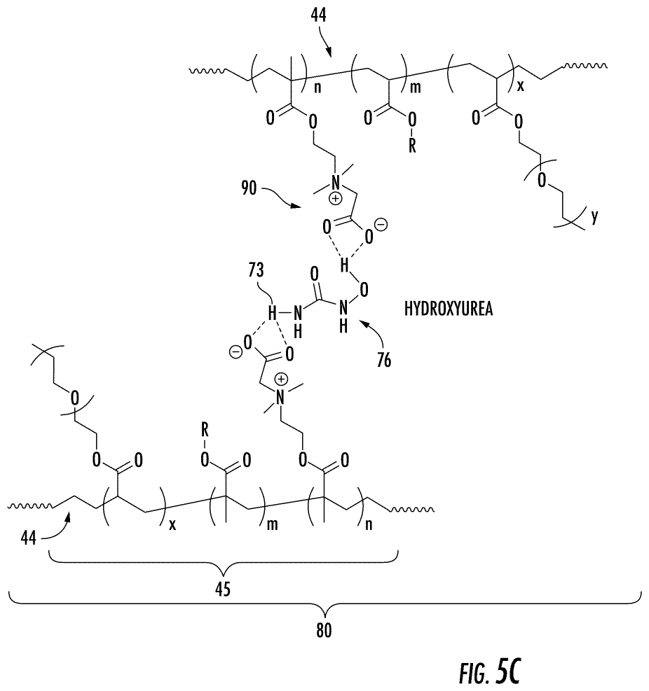

While not to held by any particular theory, it is believed that RL's having the ability to also limit interferents may be due to hydrogen bonding of polymer constituents that constitute the RL. For example, FIG. 5A is a representation of functionalized α,ω-telechelic polymer/oligomer 75 containing grafted N-vinyl pyrrolidone monomer having hydrogen bond acceptor groups 76 interacting with interferents having hydrogen donor groups 73, where an exemplary species of IL-RL polymer 43, hydrogen bonds to certain interferent molecules 70, i.e., acetaminophen, with hydrogen donor moieties 73, and interacts with hydrogen acceptor moieties 76 of grafted monomer 75 of IL-RL polymer 44 so as to form hydrogen-bonded interferent-polymer complex 77, thus preventing or reducing migration of interferent molecule 70 to the electrode and causing non-analyte current. Other reasons and rationale may exist for this observation. In examples, to achieve interferent blocking (particularly of acetaminophen), the active functionality of IL-RL polymer 44 has sufficient hydrogen bond acceptor groups for associating with acetaminophen, which has more hydrogen bond donor groups than it does hydrogen bond acceptors.

FIG. 5B is a representation of another exemplary functionalized α,ω-telechelic polymer/oligomer 45 containing a zwitterionic moiety 90 and optionally other functional groups of monomer units m and x, where R is a straight chain or branched chain alkyl, aryl, benzyl, alkoxyl or hydrogen. In examples, x and y are nonzero comprise polyethylene glycol units, for example, the zwitterionic moiety and optionally any of the other functional groups having hydrogen bond acceptor groups interacting with interferents having hydrogen donor groups, where α,ω-telechelic polymer/oligomer 45 is shown as representing a soft segment of a IL-RL polymer 44 (e.g., a polyurethane) configured to hydrogen bond to certain interferent molecules, i.e., acetaminophen 70, with hydrogen donor moieties 73, and interacts with hydrogen acceptor moieties 76 of grafted zwitterionic moiety 75 of IL-RL polymer 44 so as to form hydrogen-bonded interferent-polymer complex 78, thus preventing or reducing migration of interferent molecule 70 to the electrode and causing non-analyte current.

FIG. 5C is a representation of functionalized α,ω-telechelic polymer/oligomer 45 containing a zwitterionic moiety 90 and optionally other functional groups, the zwitterionic moiety and optionally any of the other functional groups having hydrogen bond acceptor groups interacting with another interferent having hydrogen donor groups, where α,ω-telechelic polymer/oligomer 45 is shown as representing a soft segment of a IL-RL polymer (e.g., a polyurethane) configured to hydrogen bond to certain interferent molecules, i.e., hydroxyurea, with hydrogen donor moieties 73, and interacts with hydrogen acceptor moieties 76 of grafted zwitterionic moiety 75 of IL-RL polymer 44 so as to form hydrogen-bonded interferent-polymer complex 80, thus preventing or reducing migration of interferent molecule 76 to the electrode and causing non-analyte current.

A number of different monomers are available which contain hydrogen bond acceptor groups suitable for grafting to a soft segment precursor, e.g., α,ω-telechelic or α,ω-homotelechelic polymer or oligomer intermediates, for a TPU/TPUU as described herein. For example, as depicted below, from left to right, monomers with hydrogen bond acceptor groups suitable for grafting to a soft segment precursor include N-vinylpyrrolidone, vinyl acetate, acrylonitrile, maleic anhydride, poly(ethylene glycol) methyl ether methacrylate, 2-(dimethylamino)ethyl methacrylate, and vinyl pyridine (2-vinyl pyridine and/or 4-vinyl pyridine):

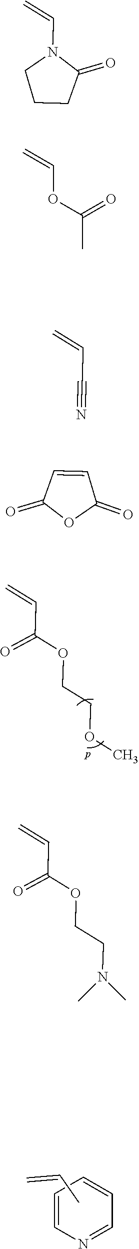

In contrast, while containing hydrogen bond acceptors, linear polyethylene glycol (PEG), a common soft segment intermediate for TPU/TPUU synthesis, has not been shown to have sufficient interferent blocking properties such that a discrete IL layer can be removed from a continuous analyte monitoring system. On the other hand, pendant, oligomeric PEG functionalities such as that seen in the PEG-ether functionalized methacrylate shown above will likely demonstrate improved binding affinity for such interferents.

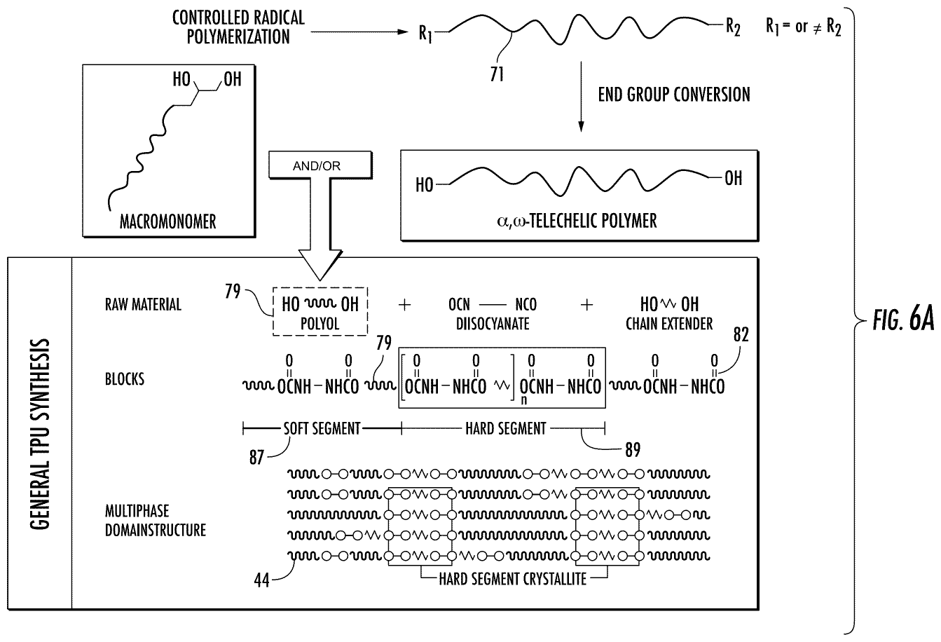

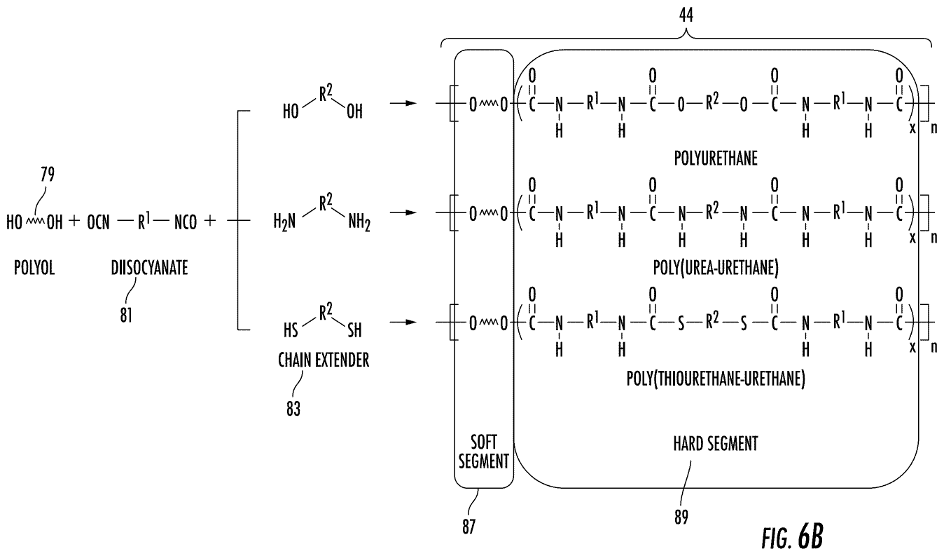

In examples, a suitable polyol average MW of 500-50000 Daltons is used for functionalization and incorporation into a TPU/TPUU as part of the soft segment of the IL-RL polymer. Once the polyol is functionalized, for example, by controlled radical polymerization (CRP) of monomer with a α,ω-telechelic or α,ω-homotelechelic polymer or oligomer (optional end group protected), it can be incorporated, after end-group deprotection, into the backbone of a TPU/TPUU as a component of the soft segment, as depicted in FIG. 6. Compared to the soft segment, the hard segment is not ideal for this functional modification as the higher crystalline nature present in hard segments will prevent small molecule diffusion, which is important for NAD(H) retention activity in ketone sensors, or analyte concentration modulation, for example.

One alternative to using CRP to synthesize a linear, functional polyol (or diamine/dithiol) would be formation of a graft copolymer using a pre-existing aliphatic diol as a scaffold. This would involve formation of radical species on the backbone of the aliphatic diol and subsequent grafting of a functional monomer to produce side-chain block segments with the intended functionalities.

### Synthesis of the Functional α,ω-Telechelic Polymer Intermediate

A generic example of preparing the functional α,ω-telechelic polymer intermediate of the present disclosure is given in the Scheme I below, depicting graft-type inclusion of functional groups into pre-existing aliphatic α,ω-telechelic di(thi)ol, where “di(thi)ol is used to include α,ω-telechelic diol, α,ω-telechelic dithiol, or a α,ω-hydroxyl-mercaptan telechelic molecule, where the di(thi)ol is optionally end group protected (PG) at step (1), grafted with a vinyl monomer after radical initiation, followed by radical termination to provide end group EG at step (2), and optionally deprotected to provide the reactive α,ω-telechelic di(thi)ol intermediate of step (3).

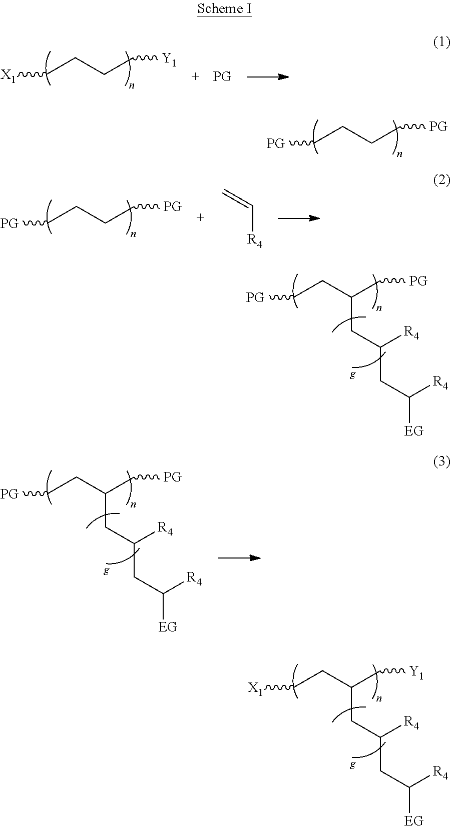

where X and Y are independently —OH or —SH, or —NH; [symbol] is linear or branched alkyl, polyether, polyester, polycarbonate or combinations thereof; g is non-zero; R4 comprises a heterocyclic group, carbonyl, a carboxylic ester, or combinations thereof; EG is an alkyl, aryl, benzyl, alkoxyl or hydrogen “end group”; and R4 comprises zwitterionic moiety, furanyl, tetrahydrofuranyl, thiophenyl, pyrrolyl, pyrrolidinyl, pyranyl, pyridinyl, pyrazinyl, pyrimidinyl, pyrrolidonyl, piperidinyl, imidazolyl, thiazolyl, dioxanyl, morpholinyl, pyrimidinyl, isoxazolyl, oxazolyl, indolyl, isoindolyl, quinolinyl, isoquinolinyl, purinyl, carbazolyl, dibenzofuranyl, chromenyl, xanthenyl, or combinations thereof.

In examples, one or more monomers that contain nitrogen heterocyclic groups are used as the vinyl monomer for grafting to the α,ω-telechelic di(thi)ol. For example, one or more monomers that contain nitrogen heterocyclic groups are shown below, from left to right, 4-vinylpyridine, 2-vinylpyridine, 4-vinylpyrimidine, 3-vinylpyridazine, 1-vinylimidazole, and 1-vinylpyrazole.

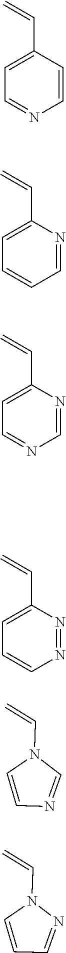

In examples improvement of the incorporation of the functional α,ω-telechelic di(thi)ol or diamine into a TPU/TPUU is achieved by providing hydroxyl chain-end functionality in the α,ω-homotelechelic polymer/oligomer with a high purity (e.g., 98-100% purity), relatively low molecular weight distribution (1 to 1.5) of the α,ω-homotelechelic polymer/oligomer, and/or low water content (e.g., ˜200-400 ppm or less). With reference to FIG. 6, α,ω-telechelic polymer or oligomer 75 is optionally end-group protected (PG) and subjected to grafting, for example, via controlled radical polymerization, and/or ROMP, and/or click chemistry, as described herein, and if needed, end group unprotected to provide for functionalized di(thi)ol or diamine.

In examples, the synthesis of these functional α,ω-telechelic polymer or oligomer is controlled/“living” radical polymerization (CRP)—also known as reversible-deactivation radical polymerization (RDRP). As used herein, CRP encompasses several different types of polymerization techniques classified by their mechanism of polymerization control, for example, atom transfer radical polymerization (ATRP); nitroxide-mediated polymerization (NMP); reversible addition-fragmentation chain transfer (RAFT) polymerization; iodine-transfer polymerization (ITP); and combinations thereof. In examples, the synthesis of the functional α,ω-telechelic polymer or oligomer is via ring-opening metathesis polymerization (ROMP)

The selection of any particular polymerization technique for preparing the functional α,ω-telechelic polymer or oligomer will depend upon many factors, including, for example, compatibility of the monomer with a particular catalyst, chain transfer agent, initiator, etc.; a target molecular weight; ability to use a hydroxyl-containing initiator and/or ability to easily convert to α,ω-telechelic polymer or oligomer end groups.

Incorporation of the Functionalized Telechelic Polymer/Oligomer into a TPU or TPUU Backbone

As shown in FIG. 6A, synthesis pathways for incorporating a diol macromonomer and/or functionalized α,ω-telechelic polymer or oligomer 71 into a TPU 82 are depicted. In examples, side-chain incorporation or “graft-through” method is used, where a diol macromolecule is reacted with diisocyanate. In examples, the diol macromolecule is prepared using a thiolgylcerol, for example, as show in Scheme II below:

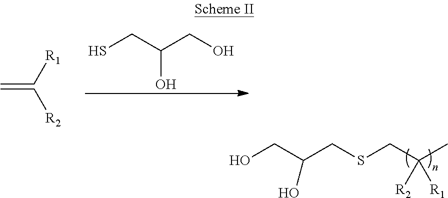

where n is nonzero; R1 is H, methyl, ethyl, or C₃-C₈ hydrocarbon; R2 is furanyl, tetrahydrofuranyl, thiophenyl, pyrrolyl, pyrrolidinyl, pyranyl, pyridinyl, pyrazinyl, pyrimidinyl, pyrrolidonyl, piperidinyl, imidazolyl, thiazolyl, dioxanyl, morpholinyl, pyrimidinyl, isoxazolyl, oxazolyl, indolyl, isoindolyl, quinolinyl, isoquinolinyl, purinyl, carbazolyl, dibenzofuranyl, chromenyl, xanthenyl, or combinations thereof. In examples, R1, R2 monomer is radical polymerized, e.g., azobisisobutyronitrile (AIBN), 60 C, in THF or THF/ethanol, and the thioglycerol is used to terminate/chain transfer the radical polymerization, providing diol macromonomer product shown in Scheme II. In other examples, polymerized R1, R2 polymer having a vinyl end group is reacted with the thioglycerol, e.g., via a Michael addition or with click chemistry providing diol macromonomer product of Scheme II. The resultant diol macromolecule can be incorporated into a backbone of TPU 82 using conventional polymer chemistry methods as shown in FIG. 6A, 6B. In examples, diol macromonomer is of an average MW of 500-50,000 Daltons.

In examples, the functionalized α,ω-telechelic polymer or oligomer is synthesized as described herein, it can be incorporated into a backbone of TPU 82 using conventional polymer chemistry methods. Diol macromonomer and/or functionalized α,ω-telechelic polymer or oligomer 79 integration with the soft segment of TPU 82 is depicted in FIG. 6B. In examples, hard segment 89 crystalline nature is minimized to maximize small molecule diffusion therethrough and therefore interferent blocking activity.

As shown in FIG. 6B, integration of diol macromonomer and/or functionalized α,ω-telechelic polymer or oligomer 79 (depicted as a diol), whereas diamines or dithiol compounds in lieu of diols are also envisaged, is used alone or in combination with chain extenders 83, which can be low molecular weight diols, diamines or dimercaptans, in reaction with a polyisocyanate 81 to provide polyurethanes, poly(urea-urethanes), or poly(thiourethane)-urethanes. In examples, polyisocyanate 81 is a prepolymer of Structure (V):

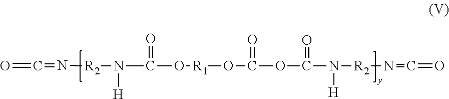

where y is a non-zero integer, where R1 is the presently disclosed functionalized telechelic polymer or oligomer as discussed herein; and R2 is the polyisocyanate residue as discussed herein. In examples, prepolymer of Structure (V) is reacted with chain extender 83 as described herein.

For example, the polyurethanes, poly(urea-urethanes), or poly(thiourethane)-urethanes incorporating the presently disclosed functionalized telechelic polymer or oligomer and optionally chain extender can be represented by structure (I):

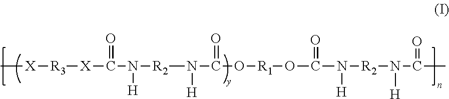

where R1 is the presently disclosed functionalized telechelic polymer or oligomer as discussed herein; R2 is the polyisocyanate residue as discussed herein, and R3 is a chain extender as discussed herein; and n is non-zero; and y represents chain extender moles. In examples, n is an average molecular weigh between 1,000 and 1,000,000 Daltons. In examples, y and n have, independently, average molecular weights of greater than 1,000 and less than 1,000,000 Daltons.

In examples, the presently disclosed IL-RL polymer has hard segments from about 15 wt. % to about 75 wt. %. In yet another example, the hard segments may be from about 25 wt. % to about 55 wt. %. In yet another example, the hard segments may be from about 35 wt. % to about 45 wt. %. For example, the presently disclosed IL-RL polymer can comprise polyurethane and/or polyurea segments and one or more of polycarbonate, polydimethylsiloxane (PDMS), polyether, fluoro-modified segments, perfluoropolyols, or polyester segments. In other examples, the presently disclosed IL-RL polymer can be a polyurethane copolymer chosen from the group including a polyether-urethane-urea, polycarbonate urethane, polyether-urethane, polyester-urethane, and/or copolymers thereof.

In examples, the hydrophilic polymer component of the presently disclosed IL-RL polymer is polyethylene oxide. For example, one useful hydrophobic-hydrophilic copolymer component is a polyurethane polymer that includes from about 1 wt. % to about 50 wt. % polyethylene oxide (PEO). In examples, the presently disclosed IL-RL polymer includes 5 wt. % to about 30 wt. % polyethylene oxide (PEO). In another example, the resistance domain includes from about 10 wt. % to about 40 wt. % PEO. The polyethylene oxide portions of the copolymer are thermodynamically driven to separate from the hydrophobic portions of the copolymer and the hydrophobic polymer component. The polyethylene oxide-based soft segment portion of the copolymer used to form the final blend affects the water pick-up and subsequent glucose permeability of the membrane.

In examples, one or more of NBDI, IPDI, TDI, MPDI, HMDI, MDI, 1,3-H6XDI, 1,4-H6XDI, CHDI, PPDI, TODI, or HDI, polyisocyanates are used to form various polyurethanes and polyurethane ureas for the IL-RL polymer and/or other sensor domains. In examples, the polyurethanes and polyurethane ureas have soft segments that are aliphatic or amphiphilic. In examples, the soft segment is comprised diol, diamine, diester, or dicarbonate in combination with the IL-RL polymer. In examples, the soft segment is comprised of a plurality of two or more of diol, diamine, diester, or dicarbonate in combination with the IL-RL polymer.

In examples, one or more of NBDI, IPDI, TDI, MPDI, HMDI, MDI, 1,3-H6XDI, 1,4-H6XDI, CHDI, PPDI, TODI, HDI, is reacted with one or more dicarbonates, polyethers, polyesters, polyalkyl-diols or polyakyl-diamines in combination with the IL-RL polymer.

In examples, one or more of NBDI, IPDI, TDI, MPDI, HMDI, MDI, 1,3-H6XDI, 1,4-H6XDI, CHDI, PPDI, TODI, HDI, is reacted with a C5 or C6 dicarbonate, in combination with the IL-RL polymer, for example U90 OXYMER™, polyhexamethylene carbonate glycol (PHA). In examples, NBDI, IPDI, TDI, MPDI, HMDI, MDI, 1,3-H6XDI, 1,4-H6XDI, CHDI, PPDI, TODI, HDI, or mixtures thereof is reacted with a C5 or C6 dicarbonate in combination with the IL-RL polymer, for example U90 OXYMER™ and one or more polyethers, polyesters, polyalkyl-diols or polyakyl-diamines. In examples, the dicarbonate is sterically branched to increase the Tg of the soft segment, for example to provide a Tg around body temperature.

In examples, one or more of hard segment diisocyantes of NBDI, IPDI, TDI, MPDI, HMDI, MDI, 1,3-H6XDI, 1,4-H6XDI, CHDI, PPDI, TODI, HDI, is reacted with the functionalized telechelic polymer alone or in combination with a polyether, for example, one or more of polytetramethylene oxide (PTMO), polypropylene oxide (PPO), polyethylene glycol (PEG), polybutadiene diol (PBU) alone or in combination with polydimethylpolysiloxane (PDMS). In examples, the same polyether of different molecular weight (Mw) is used with the functionalized telechelic polymer. In examples, two or more polyethers of the same or different Mw are used with the functionalized telechelic polymer. In examples, one or more polyethers of the same or different Mw with the functionalized telechelic polymer are used in combination with one or more PDMS polymers having the same or different Mw. While not be held to any particular theory, as the molecular weight of the soft segment decreases, phase mixing of different soft segment components increases. In examples, it has been observed that high molecular weight of the soft segment provides for the formation of rich phases, likely due to entropic contributions, among other things.

In examples, one or more of polyisocyanates NBDI, IPDI, TDI, MPDI, HMDI, MDI, 1,3-H6XDI, 1,4-H6XDI, CHDI, PPDI, TODI, HDI, is reacted with one or more polyesters in combination with the IL-RL polymer, for example, polyethylene adipate glycol (PEA), polyteramethylene adipate glycol (PBA), with the functionalized telechelic polymer alone or in combination with one or more polyethers, polyalkyl-diols or polyakyl-diamines.

In examples, NBDI, IPDI, TDI, MPDI, HMDI, MDI, 1,3-H6XDI, 1,4-H6XDI, CHDI, PPDI, TODI, HDI, or mixtures thereof is reacted with the functionalized telechelic polymer and one or more polyalkyl-diols, alone or in combination with one or more polycarbonates, polyethers, polyesters, or polyakyl-diamines.

In examples, the IL-RL polymer described above is deposited directly onto the electrode surface or onto the enzyme domain in one or more layers to yield a resistance domain thickness of from about 0.05 micron or less to about 20 microns or more. In another example, the total resistance domain thickness is from about 0.05, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns. In another example, the total resistance domain thickness is from about 2, 2.5, or 3 microns to about 3.5, 4, 4.5, or 5 microns. In some examples, the resistance domain is deposited onto the enzyme domain by spray coating or dip-coating, slot die coating, 3D printing, pico jet printing, piezo inkjet printing. In certain examples, spray coating is the deposition technique. The spraying process atomizes and mists the solution, and therefore most or all of the solvent is evaporated prior to the coating material settling on the underlying domain, thereby minimizing contact of the solvent with the enzyme. One additional advantage of spray-coating the resistance domain as described in the present disclosure includes formation of a membrane system that substantially blocks or resists ascorbate (a known electrochemical interferant in hydrogen peroxide-measuring glucose sensors). While not wishing to be bound by theory, it is believed that during the process of depositing the resistance domain as described in the present disclosure, a structural morphology is formed, characterized in that ascorbate does not substantially permeate there through.

While not be held to any particular theory, the presently disclosed TPU/TPUU with a soft segment with functionalized α,ω-telechelic polymer/oligomer is theorized to provide higher mobility of the small molecule active components, for example, NAD(H), within or into the material domain containing specified interactive functional groups and thus provide improved retention of active components of the sensing system, for example, NAD(H).

In examples, the presently disclosed IL-RL polymer includes ionic components incorporated therein to reduce the permeability of ionic interferents having the same charge as the ionic components. In another example, the presently disclosed IL-RL polymer includes a catalyst (for example, peroxidase) for catalyzing a reaction that removes or chemically modifies interferents. U.S. Pat. Nos. 6,413,396 and 6,565,509 disclose methods and materials for eliminating interfering species.

In some examples, the presently disclosed IL-RL polymer comprises one surface-active end group containing polymer, or a blend of two or more (e.g. two, three, four, five, or more) surface-active end group-containing polymers, as described above. For example, in some examples the presently disclosed IL-RL polymer comprises one surface-active end group containing polymer that comprises surface-active end groups that are zwitterionic, or are precursors or derivatives thereof. In other examples, one surface-active end group-containing polymer in a blend of two or more surface-active end group containing polymers comprises zwitterionic surface-active end groups, or precursors or derivatives thereof. In other examples, the presently disclosed IL-RL polymer comprises a blend of two or more one surface-active end group containing polymers, wherein one of the polymers in the blend comprises negatively charged surface-active end groups and one of the polymers in the blend comprises positively charged surface-active end groups.

The presently disclosed IL-RL polymer may comprise one surface-active end group containing polymer, or a blend of two or more (e.g. two, three, four, five, or more) surface-active end group-containing polymers, as described above. For example, in some examples the interference domain may comprise one surface-active end group containing polymer that comprises surface-active end groups that are zwitterionic, or are precursors or derivatives thereof. In other examples, one surface-active group containing polymer in a blend of two or more surface-active group containing polymers comprises zwitterionic surface-active groups, or precursors or derivatives thereof. In other examples, a blend may comprise a polymer with positively charged surface-active groups and a polymer with negatively charged surface-active groups.

In some examples where the presently disclosed IL-RL polymer comprises one or more zwitterionic surface-active groups, or precursors or derivatives thereof, the zwitterionic surface-active group comprises a betaine moiety such as a carboxyl, sulfo, or phosphor betaine group, or precursors or derivatives thereof (for example alkylbetaines or aminobetaines), for example up to about 0.1, 0.2, 0.5, 1, 2, or 5% wt. of the presently disclosed IL-RL polymer. Exemplary betaines include cocamidopropyl betaine, oleamidopropyl betaine, octyl sulfobetaine, caprylyl sulfobetaine, lauryl sulfobetaine, myristyl sulfobetaine, palmityl sulfobetaine, stearyl sulfobetaine, betaine (trimethylglycine), octyl betaine, phosphatidylcholine, glycine betaine, poly(carboxybetaine) (pCB), and poly(sulfobetaine) (pSB). It will be appreciated that many more zwitterionic groups, or precursors or derivatives thereof, may be applicable and that this list of exemplary betaines is not intended to limit the scope of the examples. In some examples, hydrolyzable cationic esters of zwitterionic groups (as discussed elsewhere) may be used at similar concentrations for incorporation into the presently disclosed IL-RL polymer.

In some other examples, a blend of two or more surface-active group-containing polymers comprises one surface-active group that is negatively charged and one surface-active group that is positively charged constitute the presently disclosed IL-RL polymer. In some examples, the number of negatively and positively charged surface-active groups is such that an interference domain formed from the blend is about net neutrally charged. In other examples, the number of positively charged and negatively charged surface-active groups may be unequal, with either more positively charged or negatively charged surface-active groups being present, for example, to address the charge of one or more interferents.

In certain examples, the presently disclosed IL-RL polymer may include a thin membrane that is designed to limit diffusion of certain species, for example, those greater than 34,000 daltons in molecular weight. In these examples, the presently disclosed IL-RL polymer permits certain substances (for example, hydrogen peroxide) that are to be measured by the electrodes to pass through, and prevents passage of other substances, such as potentially interfering substances. In examples, the presently disclosed IL-RL polymer is constructed of polyurethane. In an alternative example, the interference domain comprises a high oxygen soluble polymer, such as silicone.

Exemplary configurations of the presently disclosed IL-RL polymer or portions thereof are an arrangement for providing retention and recycling of NAD+ are provided. Thus, an electrode surface of a conductive wire (coaxial) or a planar conductive surface is coated with at least one layer comprising at least one enzyme as depicted in FIG. 1A. With reference to FIG. 1B, one or more optional layers may be positioned between the electrode surface and the one or more enzyme domains. For example, one or more layers of the presently disclosed IL-RL polymer can be used to reduce or eliminate signal contribution from undesirable species present, or one or more electrodes (not shown) can used to assist with wetting, system equilibrium, and/or start up. As shown in FIGS. 1A, 1B, one or more of the membranes provides a NAD+ reservoir domain providing a reservoir for NAD+. Exemplary sensor configurations can be found in U.S. Provisional Patent Application No. 63/321,340, “CONTINUOUS ANALYTE MONITORING SENSOR SYSTEMS AND METHODS OF USING THE SAME,” filed Mar. 18, 2022, and incorporated by reference in its entirety herein; and U.S. Provisional Patent Application No. 63/291,726, “MEDIATOR-TETHERED NAD(H) FOR KETONE SENSING,” filed Dec. 20, 2021, and incorporated by reference in its entirety herein.

Some examples described herein may include membranes which comprise a bioprotective domain 46 (see FIGS. 2A-2C), also referred to as a bioprotective layer, optionally including at least one polymer containing a surface-active group. In some examples, the surface-active group-containing polymer is a surface-active end group-containing polymer. In some of these examples, the surface-active end group-containing polymer is a polymer having covalently bonded surface-active end groups. However, it is contemplated that other surface-active group-containing polymers may also be used and can be formed by modification of fully-reacted base polymers via the grafting of side chain structures, surface treatments or coatings applied after membrane fabrication (e.g., via surface-modifying additives), blending of a surface-modifying additive to a base polymer before membrane fabrication, immobilization of the surface-active-group-containing soft segments by physical entrainment during synthesis, or the like. Certain exemplary bioprotective domains which may be used in some examples as described herein are described in more detail in U.S. Patent Publication No. US-2009-0247856-A1. In some examples, the surface active end groups are zwitterionic, or precursors or derivatives thereof. In some examples, the surface-active end groups are suitable to cross-link to zwitterionic compounds, precursors, or derivatives thereof.

In examples, one or more mediators that are optimal for NADH oxidation are incorporated in the one or more electrode domains or enzyme domains. In examples, organic mediators, such as phenanthroline dione, or nitrosoanilines are used. In another example, metallo-organic mediators, such as ruthenium-phenanthroline-dione or osmium(bpy)₂Cl, polymers containing covalently coupled organic mediators or organometallic coordinated mediators polymers for example polyvinylimidazole-Os(bpy)₂CI, or poly vinylpyridine-organometallic coordinated mediators (including ruthenium-phenanthroline dione) are used. Other mediators can be used as discussed further below.

In humans, serum levels of beta-hydroxybutyrate (BHB) are usually in the low micromolar range but can rise up to about 6-8 mM. Serum levels of BHB can reach 1-2 mM after intense exercise or consistent levels above 2 mM are reached with a ketogenic diet that is almost devoid of carbohydrates. Other ketones are present in serum, such as acetoacetate and acetone, however, most of the dynamic range in ketone levels is in the form of BHB. Thus, monitoring of BHB, e.g., continuous monitoring is useful for providing health information to a user or health care provider.

Thus, an exemplary continuous ketone analyte detection employing electrode-associated mediator/NAD+/dehydrogenase, for example, beta-hydroxybutyrate dehydrogenase (HBDH) for continuous monitoring of BHB using the presently disclosed IL-RL polymer is provided. In examples, a continuous ketone sensor configuration, capable of monitoring BHB, is depicted in FIG. 21A where a mediator/NAD+/dehydrogenase are present adjacent to the electrode surface 198. Alternatively, for example, multiple enzyme domains can be used in an enzyme layer, with the mediator/NAD+ comprising layer being more proximal to the electrode surface than an adjacent enzyme domain comprising the dehydrogenase enzyme. In examples, the NAD+ and/or HBDH are present in the same or different enzyme domain, and either can be immobilized, for example, using amine reactive crosslinker (e.g., glutaraldehyde, epoxides, NHS esters, imidoesters). In examples, the NAD+ is coupled to a polymer and is present in the same or different enzyme domain as HBDH. In examples, the molecular weight of NAD+ is increased to prevent or reduce migration from the sensing region, for example the NAD+ is dimerized using its C6 terminal amine with any amine-reactive crosslinker, or NAD+ is immobilized to a polymer from its C6 terminal amine. In examples, mediator polymer containing organic mediators, or organometallic coordinated mediator polymers are covalently or otherwise operably coupled to the electrode are used. In other examples, NAD+ may be electrografted to an electroactive surface (e.g., a working electrode). In examples, the electrografted NAD+ is enzyme-active. In another example, the electrografted NAD+ is not enzyme-active.

In some examples, the flux of reactant/co-reactant, such as oxygen through the sensing region has little if any effect on the transduced signal. In the configuration above, there is no consumption of oxygen or production of hydrogen peroxide, rather, direct transfer of electrons from the enzymes to the electrode surface for signal transduction. Thus, notwithstanding endogenous electroactive species such as ascorbate and urate, the need to preferentially attenuate flux of analyte relative to such other reactants such as oxygen and peroxide is reduced or eliminated. For example, homogeneous polymer which have controlled mesh size can be used. In other examples, the sensing region comprises one or more enzyme that is oxygen dependent, and oxygen flux is maximized, for example, using the presently disclosed IL-RL polymer.

### Mediators

One or more mediators can be employed to facilitate the electrolysis of one or more analytes or of a second compound that correlates with or interferes with the signal transduction of the one or more analytes. Non-polymeric and polymeric redox mediators can be used in combination with the presently disclosed IL-RL polymer.

In examples, zwitterionic compounds/polymers, Prussian blue, medola blue, methylene blue, methylene green, methyl viologen, ferrocyanide, ferrocene, cobalt ion and cobalt phthalocyanine can be used as a coating on one or more WEs to facilitate or otherwise assist in electron transfer and transduction of a detectable signal corresponding to one or more analytes. In examples, a transition metal complex is attached to one or more polymeric backbones as a redox mediator. In examples, the transition metal complexes include at least one substituted or unsubstituted biimidazole ligand. In another example, the transition metal complexes include at least one substituted or unsubstituted biimidazole ligand and a substituted or unsubstituted bipyridine or pyridylimidazole ligand.

In examples the mediator is one or more metal compounds or metal complexes of ruthenium, osmium, iron (e.g., polyvinylferrocene or hexacyanoferrate), or cobalt, including metallocene compounds thereof, for example. In examples, the mediator is coupled or otherwise bound to the conductive material of any one of the reference or working or counter electrode. In examples, non-polymeric or polymeric mediator can be adsorbed on or covalently bound to the conductive material of the electrode, such as a carbon surface or surfaces of gold, platinum, palladium, rhodium and alloys thereof. In examples, the mediator is quaternized.

A variety of methods may be used to immobilize a polymeric or non-polymeric mediator on an electrode surface, for example, adsorptive immobilization with or without cross-linking, vapor depositing, functionalization of at least a portion of the electrode surface and then chemical bonding, (ionically or covalently), of the mediator polymer to the functional groups on the electrode surface. In examples, poly(4-vinylpyridine) or poly vinylpyridine-co-styrene or polyvinylimidazoles are at least in part complexed with a transition metal compound, such as [Os(bpy)2 Cl]^{+/2+} where bpy is 2,2′-bipyridine. In examples, at least a part of the pyridine rings of the poly(4-vinylpyridine) or poly vinylpyridine-co-styrene are reacted with 2-bromoethylamine, then crosslinked, for example, using a diepoxide, such as polyethylene glycol diglycidyl ether. Other polymeric and/or non-polymeric mediators can be used, such as PVI- and PVP-ruthenium(phenanthroline dione).

Carbon surfaces can be modified for attachment of one or more polymeric and/or non-polymeric mediators, for example, by electroreduction of a diazonium salt, followed by activated by a carbodiimide, such as 1-ethyl-3-(3-dimethylaminopropyl)-carbodiimide hydrochloride then bound with an amine-functionalized mediator, such as the osmium-containing polymer described above, or 2-aminoethylferrocene, to form the mediator couple.

Similarly, gold can be functionalized by a thiol or an amine, such as cysteamine and mediator [Os(bpy)2 (pyridine-4-carboxylate)Cl]0/+ can be activated by 1-ethyl-3-(3-dimethylaminopropyl)-carbodiimide hydrochloride to form a reactive O-acylisourea that reacts with the gold-bound amine to form an amide.

### Modified Enzymes

In other examples, a genetic variant of any one of the aforementioned enzymes is used, for example, a variant that improves thermal resistance, e.g., storage or shelf stability and/or operational stability. Examples of genetic mutation to improve enzyme thermal stability include, but are not limited to addition of stabilizers, such as substrates and similar ligands, sugars, polymers, specific and non-specific ion species and small uncharged organic molecules, immobilization, protein engineering (e.g., site directed mutagenesis), and/or chemical modification. In examples, isolated enzymes from anaerobic extreme thermophiles, such as NADH oxidase isolated from Clostridium thermohydrosulfuricum, Thermus thermophilus, Thermoanaerobium brockii, Streptococcus mutans, Pyrococcus horikoshii, Bacillus licheniformis are used to impart at least some thermal operational stability, e.g. up to about 80° C., to the sensor.

### Biointerface Membrane/Layer

In examples, the sensor includes a porous material disposed over some portion thereof, which modifies the host's tissue response to the sensor. In some examples, the porous material surrounding the sensor advantageously enhances and extends sensor performance and lifetime in the short term by slowing or reducing cellular migration to the sensor and associated degradation that would otherwise be caused by cellular invasion if the sensor were directly exposed to the in vivo environment. Alternately, the porous material can provide stabilization of the sensor via tissue ingrowth into the porous material in the long term. Suitable porous materials include silicone, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), polybutylene terephthalate (PBT), polymethylmethacrylate (PMMA), polyether ether ketone (PEEK), polyamides, polyurethanes, cellulosic polymers, poly(ethylene oxide), poly(propylene oxide) and copolymers and blends thereof, polysulfones and block copolymers thereof including, for example, di-block, tri-block, alternating, random and graft copolymers, as well as metals, ceramics, cellulose, hydrogel polymers, poly(2-hydroxyethyl methacrylate, pHEMA), hydroxyethyl methacrylate, (HEMA), polyacrylonitrile-polyvinyl chloride (PAN-PVC), high density polyethylene, acrylic copolymers, nylon, polyvinyl difluoride, polyanhydrides, poly(l-lysine), poly(L-lactic acid), hydroxyethylmetharcrylate, hydroxyapatite, alumina, zirconia, carbon fiber, aluminum, calcium phosphate, titanium, titanium alloy, nitinol, stainless steel, and CoCr alloy, or the like, such as are described in U.S. Pat. No. 7,875,293 to Shults et al. and U.S. Pat. No. 7,192,450 to Brauker et al.

In some examples, the porous material surrounding the sensor provides unique advantages that can be used to enhance and extend both sensor performance and lifetime. However, such materials can also provide advantages over a period of time as well (e.g., for sensor wearability for terms of equal to or greater than 14, 15, or 21 days). Particularly, the in vivo portion of the sensor (the portion of the sensor that is implanted into the host's tissue) is encased (partially or fully) in a porous material. The porous material can be wrapped around the sensor (for example, by wrapping the porous material around the sensor or by inserting the sensor into a section of porous material sized to receive the sensor). Alternately, the porous material can be deposited on the sensor (for example, by electrospinning of a polymer directly thereon). In yet other alternate examples, the sensor is inserted into a selected section of porous biomaterial. Other methods for surrounding the in vivo portion of the sensor with a porous material can also be used as is appreciated by a person of ordinary skill in the art.

The porous material surrounding the sensor advantageously slows or reduces cellular migration to the sensor and associated degradation that would otherwise be caused by cellular invasion if the sensor were directly exposed to the in vivo environment. Namely, the porous material provides a barrier that makes the migration of cells towards the sensor more tortuous and therefore slower (providing longevity of use advantages). It is believed that this reduces or slows the sensitivity loss normally observed in a sensor over time.

In an example wherein the porous material is a high oxygen solubility material, such as porous silicone, the high oxygen solubility porous material surrounds some of or the entire in vivo portion of the sensor. In some examples, a lower ratio of oxygen-to-glucose can be sufficient to provide excess oxygen by using a high oxygen soluble domain (for example, a silicone- or fluorocarbon-based material) to enhance the supply/transport of oxygen to the enzyme domain and/or electroactive surfaces. In some examples, some signal noise normally seen by a sensor can be attributed to an oxygen deficit. Silicone has high oxygen permeability, thus promoting oxygen transport to the enzyme domain. By enhancing the oxygen supply through the use of a silicone composition, for example, glucose concentration can be less of a limiting factor. In other words, if more oxygen is supplied to the enzyme and/or electroactive surfaces, then more glucose can also be supplied to the enzyme without creating an oxygen rate-limiting excess. While not being bound by any particular theory, it is believed that silicone materials provide enhanced bio-stability when compared to other polymeric materials such as polyurethane.

In another example, the porous material further comprises a bioactive agent that releases upon insertion. In examples, the porous structure provides access for glucose permeation while allowing drug release/elution. In examples, as the bioactive agent releases/elutes from the porous structure, glucose transport may increase, for example, so as to offset any attenuation of glucose transport from the aforementioned immune response factors.

In these examples, the aforementioned porous material is a biointerface membrane comprising a first domain that includes an architecture, including cavity size, configuration, and/or overall thickness, that modifies the host's tissue response, for example, by creating a fluid pocket, encouraging vascularized tissue ingrowth, disrupting downward tissue contracture, resisting fibrous tissue growth adjacent to the device, and/or discouraging barrier cell formation. The biointerface membrane in examples covers at least the sensing mechanism of the sensor and can be of any shape or size, including uniform, asymmetrically, or axi-symmetrically covering or surrounding a sensing mechanism or sensor.

A second domain of the biointerface membrane is optionally provided that is impermeable to cells and/or cell processes. A bioactive agent is optionally provided that is incorporated into the at least one of the first domain, the second domain, the sensing membrane, or other part of the implantable device, wherein the bioactive agent is configured to modify a host tissue response. In examples, the biointerface membrane includes a bioactive agent, the bioactive agent being incorporated into at least one of the first and second domains of the biointerface membrane, or into the device and adapted to diffuse through the first and/or second domains, in order to modify the tissue response of the host to the sensor implant.

Due to the small dimension(s) of the sensor (sensing mechanism) of the present disclosure, some conventional methods of porous membrane formation and/or porous membrane adhesion are inappropriate for the formation of the biointerface membrane onto the sensor as described herein. Accordingly, the following examples exemplify systems and methods for forming and/or adhering a biointerface membrane onto a small structured sensor as defined herein. For example, the biointerface membrane or release membrane of the present disclosure can be formed onto the sensor using techniques such as electrospinning, molding, weaving, direct-writing, lyophilizing, wrapping, and the like.

In examples wherein the biointerface membrane is directly-written onto the sensor, a dispenser dispenses a polymer solution using a nozzle with a valve, or the like, for example as described in U.S. Pat. No. 7,857,756. In general, a variety of nozzles and/or dispensers can be used to dispense a polymeric material to form the woven or non-woven fibers of the biointerface membrane.

### Drug Releasing Domain-Inflammatory Response Control

A drug releasing domain that may include at least one membrane having one or more layers may be included in the membrane systems in combination with the IL-RL polymer discussed herein. In general, the inflammatory response to biomaterial implants can be divided into two phases. The first phase consists of mobilization of mast cells and then infiltration of predominantly polymorphonuclear (PMN) cells. This phase is termed the acute inflammatory phase. Over the course of days to weeks, chronic cell types that comprise the second phase of inflammation replace the PMNs. Macrophage and lymphocyte cells predominate during this phase. While not wishing to be bound by any particular theory, it is believed that restricting vasodilation and/or blocking pro-inflammatory signaling, stimulation of vascularization, or inhibition of scar formation or barrier cell layer formation, provides protection from scar tissue formation and/or reduces acute inflammation, thereby providing a stable platform for sustained maintenance of the altered foreign body response, for example.

Accordingly, bioactive intervention can modify the foreign body response in the early weeks of foreign body capsule formation and alter the long-term behavior of the foreign body capsule. Additionally, it is believed that in some circumstances the biointerface membranes and/or drug releasing membranes of the present disclosure can benefit from bioactive intervention to overcome sensitivity of the biointerface membrane and/or drug releasing membrane to implant procedure, motion of the implant, or other factors, which are known to otherwise cause inflammation, scar formation, and hinder device function in vivo.

In general, bioactive agents that are believed to modify tissue response include anti-inflammatory agents, anti-infective agents, anti-proliferative agents, anti-histamine agents, anesthetics, inflammatory agents, growth factors, angiogenic (growth) factors, adjuvants, immunosuppressive agents, antiplatelet agents, anticoagulants, ACE inhibitors, cytotoxic agents, anti-barrier cell compounds, vascularization compounds, anti-sense molecules, and the like. In some examples, bioactive agents include S1P (Sphingosine-1-phosphate), Monobutyrin, Cyclosporin A, Anti-thrombospondin-2, Rapamycin (and its derivatives), and Dexamethasone. However, other bioactive agents, biological materials (for example, proteins), or even non-bioactive substances can be incorporated into the biointerface membranes and/or drug releasing membranes of the present disclosure.

Bioactive agents suitable for use in the present disclosure are loosely organized into two groups: anti-barrier cell agents and vascularization agents. These designations reflect functions that are believed to provide short-term solute transport through the one or more membranes of the presently disclosed sensor, and additionally extend the life of a healthy vascular bed and hence solute transport through the one or more membranes long term in vivo. However, not all bioactive agents can be clearly categorized into one or other of the above groups; rather, bioactive agents generally comprise one or more varying mechanisms for modifying tissue response and can be generally categorized into one or both of the above-cited categories.

### Anti-Barrier Cell Agents

Generally, anti-barrier cell agents include compounds exhibiting effects on macrophages and foreign body giant cells (FBGCs). It is believed that anti-barrier cell agents prevent closure of the barrier to solute transport presented by macrophages and FBGCs at the device-tissue interface during FBC maturation.

Anti-barrier cell agents generally include mechanisms that inhibit foreign body giant cells and/or occlusive cell layers. For example, Super Oxide Dismutase (SOD) Mimetic, which utilizes a manganese catalytic center within a porphyrin like molecule to mimic native SOD and effectively remove superoxide for long periods, thereby inhibiting FBGC formation at the surfaces of biomaterials in vivo, is incorporated into a biointerface membrane or release membrane.

Anti-barrier cell agents can include nano or micro structures, anti-inflammatory and/or immunosuppressive mechanisms that affect early FBC formation. Cyclosporine, which stimulates very high levels of neovascularization around biomaterials, can be incorporated into a biointerface membrane (see U.S. Pat. No. 5,569,462 to Martinson et al.), or release membrane.

In examples, pilocarpine, dexamethasone, dexamethasone salts, or dexamethasone derivatives in particular, dexamethasone acetate, which, for example, abates the intensity of the FBC response at the device-tissue interface, is incorporated into the drug releasing membrane in combination with the IL-RL polymer. In another example, a combination of dexamethasone and dexamethasone acetate is incorporated into the drug releasing membrane in combination with the IL-RL polymer. In another example, dexamethasone and/or dexamethasone acetate combined with one or more other anti-inflammatory and/or immunosuppressive agents is incorporated into the drug releasing membrane in combination with the IL-RL polymer. Alternately, Rapamycin, which is a potent specific inhibitor of some macrophage inflammatory functions, can be incorporated into the release membrane in combination with the IL-RL polymer alone or in combination with dexamethasone, dexamethasone salts, or dexamethasone derivatives, in particular, dexamethasone acetate.

Other suitable medicaments, pharmaceutical compositions, therapeutic agents, or other desirable substances can be incorporated into the drug releasing membrane in combination with the IL-RL polymer of the present disclosure, including, but not limited to, anti-inflammatory agents, anti-infective agents, necrosing agents, and anesthetics.

Generally, anti-inflammatory agents reduce acute and/or chronic inflammation adjacent to the implant, in order to decrease the formation of an FBC to reduce or prevent barrier cell layer formation. Suitable anti-inflammatory agents include but are not limited to, for example, nonsteroidal anti-inflammatory drugs (NSAIDs) such as acetaminophen, aminosalicylic acid, aspirin, celecoxib, choline magnesium trisalicylate, diclofenac potassium, diclofenac sodium, diflunisal, etodolac, fenoprofen, flurbiprofen, ibuprofen, indomethacin, interleukin (IL)-10, IL-6 mutein, anti-IL-6 iNOS inhibitors (for example, L-NAME or L-NMDA), Interferon, ketoprofen, ketorolac, leflunomide, melenamic acid, mycophenolic acid, mizoribine, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, paclitaxel, tacrolimus, tranilast, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, pilocarpine, dexamethasone, dexamethasone salts, dexamethasone derivatives, and dexamethasone acetate.

Generally, immunosuppressive and/or immunomodulatory agents interfere directly with several key mechanisms necessary for involvement of different cellular elements in the inflammatory response. Suitable immunosuppressive and/or immunomodulatory agents include anti-proliferative, cell-cycle inhibitors, (for example, paclitaxol (e.g., Sirolimus), cytochalasin D, infiximab), taxol, actinomycin, mitomycin, thospromote VEGF, estradiols, NO donors, QP-2, tacrolimus, tranilast, actinomycin, everolimus, methothrexate, mycophenolic acid, angiopeptin, vincristing, mitomycine, statins, C MYC antisense, sirolimus (and analogs), RestenASE, 2-chloro-deoxyadenosine, PCNA Ribozyme, batimstat, prolyl hydroxylase inhibitors, PPARy ligands (for example troglitazone, rosiglitazone, pioglitazone), halofuginone, C-proteinase inhibitors, probucol, BCP671, EPC antibodies, catchins, glycating agents, endothelin inhibitors (for example, Ambrisentan, Tesosentan, Bosentan), Statins (for example, Cerivasttin), E. coli heat-labile enterotoxin, and advanced coatings.

Generally, anti-infective agents are substances capable of acting against infection by inhibiting the spread of an infectious agent or by killing the infectious agent outright, which can serve to reduce immuno-response without inflammatory response at the implant site. Anti-infective agents include, but are not limited to, anthelmintics (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin; clindamycin; colistimethate sodium; polymyxin b sulfate; vancomycin; antivirals including acyclovir, amantadine, didanosine, efavirenz, foscarnet, ganciclovir, indinavir, lamivudine, nelfinavir, ritonavir, saquinavir, silver, stavudine, valacyclovir, valganciclovir, zidovudine; quinolones (ciprofloxacin, levofloxacin); sulfonamides (sulfadiazine, sulfisoxazole); sulfones (dapsone); furazolidone; metronidazole; pentamidine; sulfanilamidum crystallinum; gatifloxacin; and sulfamethoxazole/trimethoprim.

Generally, necrosing agents are any drug that causes tissue necrosis or cell death. Necrosing agents include cisplatin, BCNU, taxol or taxol derivatives, and the like.

### Vascularization Agents

Generally, vascularization agents include substances with direct or indirect angiogenic properties. In some cases, vascularization agents may additionally affect formation of barrier cells in vivo. By indirect angiogenesis, it is meant that the angiogenesis can be mediated through inflammatory or immune stimulatory pathways. It is not fully known how agents that induce local vascularization indirectly inhibit barrier-cell formation; however, it is believed that some barrier-cell effects can result indirectly from the effects of vascularization agents.

Vascularization agents include mechanisms that promote neovascularization around the sensing membrane and/or minimize periods of ischemia by increasing vascularization close to the device-tissue interface. Sphingosine-1-Phosphate (S1P), which is a phospholipid possessing potent angiogenic activity, is incorporated into a biointerface membrane in combination with the IL-RL polymer. Monobutyrin, which is a potent vasodilator and angiogenic lipid product of adipocytes, is incorporated into a biointerface membrane or release membrane. In another example, an anti-sense molecule (for example, thrombospondin-2 anti-sense), which increases vascularization, is incorporated into a biointerface membrane in combination with the IL-RL polymer.

Vascularization agents can include mechanisms that promote inflammation, which is believed to cause accelerated neovascularization in vivo. In examples, a xenogenic carrier, for example, bovine collagen, which by its foreign nature invokes an immune response, stimulates neovascularization, and is incorporated into a biointerface membrane in combination with the IL-RL polymer of the present disclosure. In another example, Lipopolysaccharide, which is a potent immunostimulant, is incorporated into a biointerface membrane in combination with the IL-RL polymer. In another example, a protein, for example, a bone morphogenetic protein (BMP), which is known to modulate bone healing in tissue, is incorporated into a biointerface membrane in combination with the IL-RL polymer.

Generally, angiogenic agents are substances capable of stimulating neovascularization, which can accelerate and sustain the development of a vascularized tissue bed at the device-tissue interface. Angiogenic agents include, but are not limited to, copper ions, iron ions, tridodecylmethylammonium chloride, Basic Fibroblast Growth Factor (bFGF), (also known as Heparin Binding Growth Factor-Il and Fibroblast Growth Factor II), Acidic Fibroblast Growth Factor (aFGF), (also known as Heparin Binding Growth Factor-I and Fibroblast Growth Factor-I), Vascular Endothelial Growth Factor (VEGF), Platelet Derived Endothelial Cell Growth Factor BB (PDEGF-BB), Angiopoietin-1, Transforming Growth Factor Beta (TGF-Beta), Transforming Growth Factor Alpha (TGF-Alpha), Hepatocyte Growth Factor, Tumor Necrosis Factor-Alpha (TNF-Alpha), Placental Growth Factor (PLGF), Angiogenin, Interleukin-8 (IL-8), Hypoxia Inducible Factor-I (HIF-1), Angiotensin-Converting Enzyme (ACE) Inhibitor Quinaprilat, Angiotropin, Thrombospondin, Peptide KGHK, Low Oxygen Tension, Lactic Acid, Insulin, Copper Sulphate, Estradiol, prostaglandins, cox inhibitors, endothelial cell binding agents (for example, decorin or vimentin), glenipin, hydrogen peroxide, nicotine, and Growth Hormone.

Generally, pro-inflammatory agents are substances capable of stimulating an immune response in host tissue, which can accelerate or sustain formation of a mature vascularized tissue bed. For example, pro-inflammatory agents are generally irritants or other substances that induce chronic inflammation and chronic granular response at the implantation-site. While not wishing to be bound by theory, it is believed that formation of high tissue granulation induces blood vessels, which supply an adequate or rich supply of analytes to the device-tissue interface. Pro-inflammatory agents include, but are not limited to, xenogenic carriers, Lipopolysaccharides, S. aureus peptidoglycan, and proteins.

The bioactive agents can be spatially distributed or dispersed throughout the drug releasing membrane where the spatial distribution or dispersion can be uniform or nonuniform, and/or vary vertically and/or horizontally in a gradient. Other substances that can be incorporated into membranes of the present disclosure include various pharmacological agents, excipients, and other substances suitable for use in pharmaceutical formulations.

Although the bioactive agent in some examples is incorporated into the biointerface membrane in combination with the IL-RL polymer and/or implantable device, in other examples the bioactive agent can be administered concurrently with, prior to, or after implantation of the device systemically, for example, by oral administration, or locally, for example, by subcutaneous injection near the implantation site, or transdermally via the adhesive used to secure the analyte sensor to the host. Thus, a depot of bioactive agent can be present in the adhesive or the analyte sensor housing for passive or post-controlled release of the bioactive agent during use of the analyte sensor.

In examples, the drug releasing membrane functions as a biointerface membrane in combination with the IL-RL polymer. In another example, the drug releasing membrane is chemically distinct from the biointerface membrane. In this example, one or more bioactive agents can be incorporated into each of the biointerface membrane and the drug releasing membrane. In this example, the biointerface membrane and the drug releasing membrane may each include the same bioactive agent(s). In other examples, the biointerface membrane and the drug releasing membrane may each independently include one or more bioactive agents that differ in, for example, chemistry, loading (wt. %) of the respective membrane, or other factors or combinations of factors. In another example membrane system, a drug releasing membrane is present but no biointerface membrane is used. In such examples, one or more bioactive agents are incorporated into the drug releasing membrane. Generally, numerous variables can affect the pharmacokinetics of bioactive agent release. The bioactive agents of the present disclosure can be configured for short- and/or long-term release. In some examples, the bioactive agents are designed to aid or overcome factors associated with short-term effects (for example, acute inflammation) of the foreign body response, which can begin as early as the time of implantation and extend up to about one month after implantation. In some examples, the bioactive agents of the present disclosure are designed to aid or overcome factors associated with long-term effects, for example, chronic inflammation, barrier cell layer formation, or build-up of fibrotic tissue of the foreign body response, which can begin as early as about one week after implantation and extend for the life of the implant, for example, months to years. In some examples, the bioactive agents of the present disclosure combine short- and long-term release to exploit the benefits of both. U.S. Pat. No. 7,875,293 to Shults et al., U.S. Provisional Applications 63/318,901, filed Mar. 11, 2022, and U.S. patent application Ser. No. 17/945,585, filed Sep. 15, 2020 discloses a variety of systems and methods for release of the bioactive agents, the discloses of which are incorporated by reference herein.

### Continuous Analyte Sensor

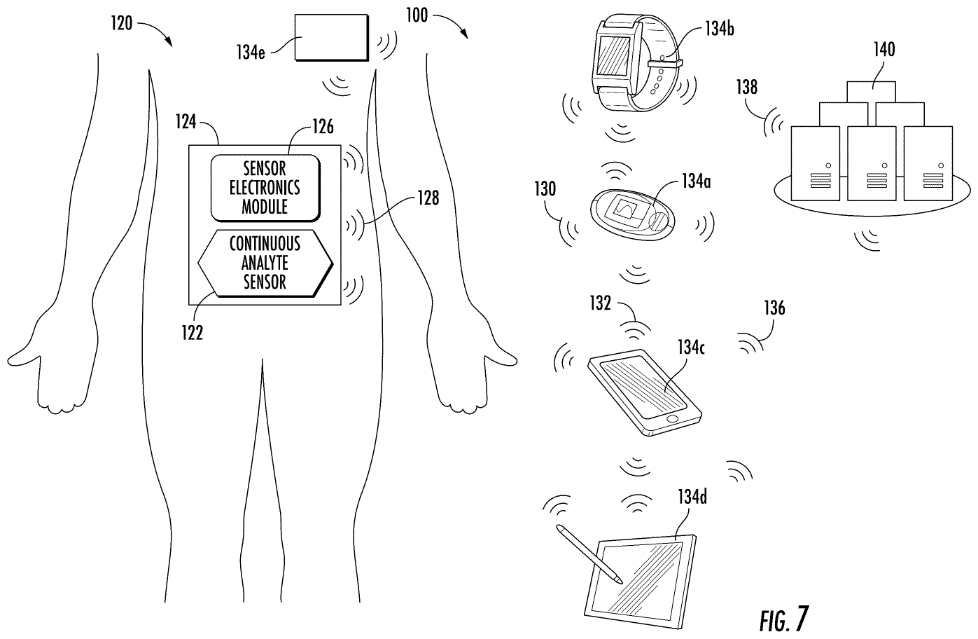

FIG. 7 is a diagram depicting an example continuous analyte monitoring system configured to measure one or more analytes and/or electrophysiological indicators (e.g., blood pressure, heart rate, core temperature, etc.) as discussed herein. The monitoring system includes a continuous sensor system 124 operatively connected to a host 120 and a plurality of display devices 134 a-e according to certain aspects of the present disclosure. It should be noted that display device 134e alternatively or in addition to being a display device, may be a medicament delivery device that can act cooperatively with the continuous analyte sensor system 124 to deliver medicaments to host 120. The continuous sensor system 124 may include a sensor electronics module 126 and a continuous analyte sensor 122 associated with the sensor electronics module 126. The sensor electronics module 126 may be in direct wireless communication with one or more of the plurality of the display devices 134a-e via wireless communications signals. In examples, display devices 134a-e may also communicate amongst each other and/or through each other to continuous analyte sensor system 124. For ease of reference, wireless communications signals from continuous analyte sensor system 124 to display devices 134a-e can be referred to as “uplink” signals 128. Wireless communications signals from, e.g., display devices 134a-e to continuous analyte sensor system 124 can be referred to as “downlink” signals 130. Wireless communication signals between two or more of display devices 134a-e may be referred to as “crosslink” signals 132. Additionally, wireless communication signals can include data transmitted by one or more of display devices 134a-d via “long-range” uplink signals 136 (e.g., cellular signals) to one or more remote servers 140 or network entities, such as cloud-based servers or databases, and receive long-range downlink signals 138 transmitted by remote servers 140.

The sensor electronics module 126 includes sensor electronics that are configured to process sensor information and generate transformed sensor information. In certain examples, the sensor electronics module 126 includes electronic circuitry associated with measuring and processing data from continuous analyte sensor 122, including prospective algorithms associated with processing and calibration of the continuous analyte sensor data. The sensor electronics module 126 can be integral with (non-releasably attached to) or releasably attachable to the continuous analyte sensor 122 achieving a physical connection therebetween. The sensor electronics module 126 may include hardware, firmware, and/or software that enables analyte level measurement. For example, the sensor electronics module 126 can include a potentiostat, a power source for providing power to continuous analyte sensor 122, other components useful for signal processing and data storage, and a telemetry module for transmitting data from itself to one or more display devices 134a-e. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381,2008/0033254,2005/0203360,2005/0154271,2005/0192557,2006/0222566, 2007/0203966 and 2007/0208245, each of which are incorporated herein by reference in their entirety for all purposes.

Display devices 134a-e are configured for displaying, alarming, and/or basing medicament delivery on the sensor information that has been transmitted by the sensor electronics module 126 (e.g., in a customized data package that is transmitted to one or more of display devices 134a-e based on their respective preferences). Each of the display devices 134a-e can include a display such as a touchscreen display for displaying sensor information to a user (most often host 120 or a caretaker/medical professional) and/or receiving inputs from the user. In some examples, the display devices 134a-e may include other types of user interfaces such as a voice user interface instead of or in addition to a touchscreen display for communicating sensor information to the user of the display device 134a-e and/or receiving user inputs. In some examples, one, some or all of the display devices 134a-e are configured to display or otherwise communicate the sensor information as it is communicated from the sensor electronics module 126 (e.g., in a data package that is transmitted to respective display devices 134a-e), without any additional prospective processing required for calibration and real-time display of the sensor information.

In the example of FIG. 7, one of the plurality of display devices 134a-e may be a custom display device 134a specially designed for displaying certain types of displayable sensor information associated with analyte values received from the sensor electronics module 126 (e.g., a numerical value and an arrow, in some examples). In some examples, one of the plurality of display devices 134a-e may be a handheld device 134c, such as a mobile phone based on the Android, iOS operating system or other operating system, a palm-top computer and the like, where handheld device 134c may have a relatively larger display and be configured to display a graphical representation of the continuous sensor data (e.g., including current and historic data). Other display devices can include other hand-held devices, such as a tablet 134d, a smart watch 134b, a medicament delivery device 134e, a blood glucose meter, and/or a desktop or laptop computers.

As alluded to above, because the different display devices 134a-e provide different user interfaces, content of the data packages (e.g., amount, format, and/or type of data to be displayed, alarms, and the like) can be customized (e.g., programmed differently by the manufacture and/or by an end user) for each particular display device and/or display device type. Accordingly, in the example of FIG. 15, one or more of display devices 134a-e can be in direct or indirect wireless communication with the sensor electronics module 126 to enable a plurality of different types and/or levels of display and/or functionality associated with the sensor information, which is described in more detail elsewhere herein.

Generally, continuous analyte sensor 122 may be an implantable analyte sensor that utilizes amperometric electrochemical sensor technology to measure an analyte concentration. Electrodes comprising continuous analyte sensor 122 may include a working electrode, a counter electrode, and a reference electrode. In examples, the counter electrode is provided to balance the current generated by the species being measured at the working electrode.

In some alternative examples, additional electrodes can be included within the assembly, for example, a three-electrode system (working, reference, and counter electrodes) and an additional working electrode (e.g., an electrode which can be used to generate oxygen, which is configured as a baseline subtracting electrode, or which is configured for measuring additional analytes, including but not limited to the analytes discussed herein). U.S. Pat. No. 7,081,195, U.S. Patent Publication No. 2005/0143635 and U.S. Patent Publication No. 2007/0027385, each of which are incorporated herein by reference in its entirety, describe some systems and methods for implementing and using additional working, counter, and reference electrodes.

### Sensor Electronics

The following description of electronics associated with the sensor is applicable to a variety of continuous multi-analyte sensors, such as non-invasive, minimally invasive, and/or invasive (e.g., transcutaneous and implantable) sensors. For example, the sensor electronics and data processing as well as the receiver electronics and data processing described below can be incorporated into the implantable glucose sensor disclosed in U.S. Pat. Appl. Pub. No. 2005/0245799 to Brauker et al. and U.S. Pat. Appl. Pub. No. 2006/0015020 to Neale et al.

The high-fidelity readout of analytes using electrochemical methods uses specialized hardware. Application of a stable prescribed bias potential is used since the redox reaction of interest is driven by the bias in an exponential fashion, according to the Butler-Volmer relation. Moreover, owing to the minute levels of current encountered in amperometric, voltametric, and impedimetric in vivo analyte sensors, oftentimes in the picoampere range, high fidelity analog circuit design techniques is employed to quantize these current levels with femtoampere-level resolution. Thus, in examples, a precision bias circuit, known as a potentiostat, is implemented, along with a transimpedance amplifier offering high transconductance. In examples the combination of a precision bias circuit/potentiostat, along with a high transconductance transimpedance amplifier, collectively, is known as an amperometric (or potentiostatic) analog front end (AFE).

In examples, a high-resolution analog-to-digital converter is paired with this AFE system to quantize signals with sufficient resolution (e.g., 16 bits with high accuracy and low nonlinearity figures-of-merit). In examples, a poteniotstatic AFE comprising an ultra-high input impedance (Zin >100 GΩ) at the reference electrode input and an ultra-low input bias current (ibias)/input offset voltage (Voffset) (ibias <100 pA, Voffset <10 mV) at the working electrode input can be used. The counter electrode terminal, in the case of a 3-terminal electrochemical sensing arrangement, in examples, provides sufficient compliance voltage to enable the potentiostat to source (or sink) a sufficient amount of current to sustain a redox reaction of interest at the working electrode. In examples, CMOS input instrumentation amplifiers, unity gain amplifiers, and differential amplifiers are used within the said circuits to achieve these performance requirements. A system that employs multiple potentiostats for the measurement of multiple analytes, can be configured to minimize of cross-talk between sensing channels (e.g., channel-to-channel isolation >75 dB). In examples, guard rings are also used in potentiostat designs to minimize noise that might result from high input impedance circuit couplings and thereby corrupt accurate low current measurements.

An exemplary dual potentiostat precision electrochemical analog front end commercially available from Analog Devices® is the Maxim Integrated® MAX30132. This integrated circuit is used in blood glucose meters and can be extended to the quantification of other analytes. The MAX30132 comprises guard rings and temperature compensation and is able to measure two analytes in parallel.

Guard rings are employed, in examples, to complement the design of a dual-channel AFE and to reduce capacitively-coupled noise that can perturb sensitive measurements of charge, e.g., when the charge magnitude is low. Moreover, noise can also be coupled between both channels, resulting in measurable signal cross-talk. Recalling from the canonical relation for charge stored in a capacitive system:

$$Q = CV$$

The classic interpretation of this relation if voltage changes with time is often given by:

$$\frac{dQ ( t )}{dt} = i ( t ) = C \frac{dV ( t )}{dt}$$

The above relation may not account for time-dependent perturbations of the capacitive nature of the system. Thus, the product rule to form the following differential equation can be used:

$$\frac{dQ ( t )}{dt} = i ( t ) = \frac{dC ( t )}{dt} V ( t ) + C ( t ) \frac{dV ( t )}{dt}$$

Thus, a measured change in electrical charge, otherwise known as a current, is not only sensitive to a variation in electrical potential, but it is also affected due to change in the capacitive energy storage characteristics of the system (capacitive coupling). In examples, by driving a circumscribed conductor to the same potential as a signal trace on a PCB, leakage current is minimized, as the Ohmic drop between the conductors trends towards 0. For example, low leakage current can be achieved using a voltage buffer/follower that matches the guard voltage to the signal voltage, or in low-voltage differential sensing with an instrumentation amplifier, the common-mode voltage. The leakage from the guard ring to other circuit elements is typically of little concern as it is being sourced from a buffer which has a low output impedance.

In examples an additional sensing channel can be incorporated in an existing form-factor, e.g., a continuous glucose sensor (CGM) to enable the quantification of two metabolites, continuously and in real-time. Other form factors for the additional sensing channel can be used.

In examples, a suitable dual-channel amperometric analog front end for inclusion into a continuous analyte monitor wearable is used. Such a dual channel comprising a viable circuit topology meeting established performance requirements to achieve continuous amperometric quantification of two analytes is employed. In some examples, an amperometric analog front end (AFE)/bi-potentiostat amenable to power budget and size/footprint considerations is chosen. In other examples, an amperometric analog front end may feature an input for an external temperature sensor (e.g., thermistor or RTD) or may otherwise contain an internal bandgap-based temperature sensor. The reading from said temperature sensor may be used to compensate for increasing rates of enzyme turnover with temperature, in accordance with the Arrhenius relation.

In another example, alone or in combination with the above exemplary analog front end (AFE), a printed circuit board (PCB) design and/or printed circuit board assembly (PCBA) supporting the dual-channel amperometric AFE and the dual coaxial wire sensing construct is provided. In examples, the PCBA is populated with the necessary components for a functional transmitter with dual-channel amperometric sensing capability. PCB design can incorporate an effective guard ring topology enabling low-noise amperometric measurements and can feature a common reference/counter electrode configuration for both sensing channels.

In examples, alone or in combination with the PCB, PCBA, AFE, the dual-wire coaxial sensor construct is adapted to (e.g., embedded into) an existing CGM form-factor or another form factor. In another example, the selection of materials for the form-factor can be used to mitigate ingress of water, dust, and/or light. The said materials can also be selected to present low outgassing or vapor/water uptake figures of merit. The said materials can also be selected to possess high linear, sheet, or volume resistivity figures of merit (ohm-cm) to eliminate leakage currents or signal cross-talk between channels and electrodes.

In another example, alone or in combination with the above AFE, the printed circuit board (PCB) design and printed circuit board assembly (PCBA) supporting the dual-channel amperometric AFE and the dual coaxial wire sensing construct is coupled with firmware supporting dual-channel amperometric measurements at configurable data storage intervals. In examples, existing firmware of a continuous analyte monitor is modified to enable power-efficient control of the amperometric AFE and data transmission, for example, over a Bluetooth Low Energy connection. In another example, the firmware modification of existing firmware to enable extended data storage in an embedded flash memory IC and transmission of readings to a real-time display is provided.

Thus, in examples, a power-efficient firmware enabling dual-channel amperometric measurements at pre-defined data storage intervals, with amperometric measurements (both channels) recordable in an embedded device memory for at least the full intended operational lifetime (15 days) of the transmitter is provided.

In examples, the dual-channel amperometric sensing coupled to the transmitter PCBA paired with a dual coaxial wire sensor configuration in vitro, provides data capable of being logged in embedded device memory and transmitted to a paired real-time display for at least 7 days, 8 days, 9 days, 10 days, 11 days, 12 days, 13 days, 14 days, 15 days, 21 days, or over 21 days.

In examples, the dual analyte transmitter, paired with a dual coaxial sensor configuration measuring two channels of glucose information, in an animal model, shows 15.5-day quantitative tracking of interstitial glucose levels, as assessed by a comparator measure in arterialized blood samples.

In examples, the amperometric analog front end supporting the multi-analyte sensing platform supports one or more potentiostats, temperature correction, configurable bias conditions, and advanced electrochemistry for multi-analyte sensing. In examples, guard ring topology enabling low-noise amperometric measurements is employed.

By way of example, the multi-analyte transmitter, paired with a dual coaxial sensor contingent measuring two channels of glucose information, in an animal model, is likely to show 15.5-day quantitative tracking of interstitial glucose levels, as assessed by a comparator measure in arterialized blood samples. In examples, the dual coaxial sensor configuration can be tested using glucose with another analyte, e.g., ketone.

In some examples, the potentiostat includes a resistor that translates the current into voltage. In some alternate examples, a current to frequency converter is provided that is configured to continuously integrate the measured current, for example, using a charge counting device. An A/D converter digitizes the analog signal into a digital signal, also referred to as “counts” for processing. Accordingly, the resulting raw data stream in counts, also referred to as raw sensor data, is directly related to the current measured by the potentiostat.

A processor module includes the central control unit that controls the processing of the sensor electronics. In some examples, the processor module includes a microprocessor, however a computer system other than a microprocessor can be used to process data as described herein, for example an ASIC can be used for some or all of the sensor's central processing. The processor typically provides semi-permanent storage of data, for example, storing data such as sensor identifier (ID) and programming to process data streams (for example, programming for data smoothing and/or replacement of signal artifacts such as is described in U.S. Pat. No. 8,010,174 to Goode et al. The processor additionally can be used for the system's cache memory, for example for temporarily storing recent sensor data. In some examples, the processor module comprises memory storage components such as ROM, RAM, dynamic-RAM, static-RAM, non-static RAM, EEPROM, rewritable ROMs, flash memory, or the like.

In some examples, the processor module comprises a digital filter, for example, an IIR or FIR filter, configured to smooth the raw data stream from the A/D converter. Generally, digital filters are programmed to filter data sampled at a predetermined time interval (also referred to as a sample rate). In some examples, wherein the potentiostat is configured to measure the analyte at discrete time intervals, these time intervals determine the sample rate of the digital filter. In some alternate examples, wherein the potentiostat is configured to continuously measure the analyte, for example, using a current-to-frequency converter as described above, the processor module can be programmed to request a digital value from the A/D converter at a predetermined time interval, also referred to as the acquisition time. In these alternate examples, the values obtained by the processor are advantageously averaged over the acquisition time due the continuity of the current measurement. Accordingly, the acquisition time determines the sample rate of the digital filter. In examples, the processor module is configured with a programmable acquisition time, namely, the predetermined time interval for requesting the digital value from the A/D converter is programmable by a user within the digital circuitry of the processor module. In examples, an acquisition time of from about 2 seconds to about 512 seconds is used; however, any acquisition time can be programmed into the processor module. A programmable acquisition time is advantageous in maximizing the usefulness of, for example, noise filtration, time lag, and/or processing/battery power.

In some examples, the processor module is configured to build the data packet for transmission to an outside source, for example, an RF transmission to a receiver as described in more detail below. Generally, the data packet comprises a plurality of bits that can include a sensor ID code, raw data, filtered data, and/or error detection or correction. The processor module can be configured to transmit any combination of raw and/or filtered data.

In some examples, the processor module further comprises a transmitter portion that is programmable and programmed for a transmission interval of the sensor data to a receiver, or the like. In some examples, the transmitter portion, which is programmable for the interval of transmission. In one such example, a coefficient can be chosen (e.g., a number of from about 1 to about 100, or more), wherein the coefficient is multiplied by the acquisition time (or sampling rate), such as described above, to define the transmission interval of the data packet. Thus, in some examples, the transmission interval is programmable between about 2 seconds and about 850 minutes. In further examples, the transmission interval is programmable between about 30 second and 5 minutes. Nevertheless, any transmission interval can be programmable or programmed into the processor module. A variety of alternate systems and methods for providing a programmable transmission interval can also be employed. By providing a programmable transmission interval, data transmission can be customized to meet a variety of design criteria (e.g., reduced battery consumption, timeliness of reporting sensor values, etc.). In some examples, a transceiver module can also be included in the sensor electronics. In examples, the transceiver module may be configured to transmit and/or receive sensor data.

The various memories and/or memory of the processor unit(s) and/or storage device may store one or more sets of instructions and data structures (e.g., instructions) embodying or used by any one or more of the methodologies or functions described herein. These instructions, when executed by processor unit(s) cause various operations to implement the disclosed examples. The instructions can further be transmitted or received over a communications network using a transmission medium via the network interface device using any one of several well-known transfer protocols (e.g., HTTP). Examples of communication networks include a LAN, a WAN, the Internet, mobile telephone networks, plain old telephone service (POTS) networks, and wireless data networks (e.g., Wi-Fi, 3G, 4G LTE/LTE-A, 5G, or WiMAX networks). The term “transmission medium” shall be taken to include any intangible medium that is capable of storing, encoding, or carrying instructions for execution by the machine, and includes digital or analog communications signals or other intangible media to facilitate communication of such software.

Conventional glucose sensors measure current in the nanoAmp range. In contrast to conventional glucose sensors, the presently disclosed multi-analyte sensors are configured to measure the current flow in the picoAmp range, and in some examples, femtoAmps, if required. Namely, for every unit (mg/dL) of glucose measured, at least one picoAmp of current is measured. In some examples, the analog portion of the A/D converter is configured to continuously measure the current flowing at the working electrode and to convert the current measurement to digital values representative of the current. In examples, the current flow is measured by a charge counting device (e.g., a capacitor). Thus, a signal is provided, whereby a high sensitivity maximizes the signal received by a minimal amount of measured hydrogen peroxide (e.g., minimal glucose requirements without sacrificing accuracy even in low glucose ranges), reducing the sensitivity to oxygen limitations in vivo (e.g., in oxygen-dependent glucose sensors).

A battery is operably connected to the sensor electronics and provides the power for the sensor. In examples, the battery is a lithium manganese dioxide battery; however, any appropriately sized and powered battery can be used (for example, AAA, nickel-cadmium, zinc-carbon, alkaline, lithium, nickel-metal hydride, lithium-ion, zinc-air, zinc-mercury oxide, silver-zinc, and/or hermetically-sealed). In some examples, the battery is rechargeable, and/or a plurality of batteries can be used to power the system. The sensor can be transcutaneously powered via an inductive coupling, for example. In some examples, a quartz crystal is operably connected to the processor and maintains system time for the computer system as a whole, for example, for the programmable acquisition time within the processor module.

An optional temperature probe can be provided, wherein the temperature probe is located on the electronics assembly or the glucose sensor itself. The temperature probe can be used to measure ambient temperature in the vicinity of the glucose sensor. This temperature measurement can be used to add temperature compensation to the calculated glucose value.

An RF module is operably connected to the processor and transmits the sensor data from the sensor to a receiver within a wireless transmission via antenna or other wireless communication methods. In some examples, a second quartz crystal provides the time base for the RF carrier frequency used for data transmissions from the RF transceiver. In some alternate examples, however, other mechanisms, such as optical, infrared radiation (IR), ultrasonic, or the like, can be used to transmit and/or receive data.

In the RF telemetry module of the present disclosure, the hardware and software are designed for low power requirements to increase the longevity of the device (for example, to enable a life of from about 3 to about 24 months, or more) with maximum RF transmittance from the in vivo environment to the ex vivo environment for some implantable sensors (for example, a distance of from about one to ten meters or more). In some examples, a high frequency carrier signal of from about 402 MHz to about 433 MHz is employed in order to maintain lower power requirements. Additionally, in some implantable devices, the carrier frequency is adapted for physiological attenuation levels, which is accomplished by tuning the RF module in a simulated in vivo environment to ensure RF functionality after implantation; accordingly, the glucose sensor can sustain sensor function for 3 months, 6 months, 12 months, or 24 months or more.

In some examples, output signal (from the sensor electronics) is sent to a receiver (e.g., a computer or other communication station). The output signal is typically a raw data stream that is used to provide a useful value of the measured analyte concentration to a patient or a doctor, for example. In some examples, the raw data stream can be continuously or periodically algorithmically smoothed or otherwise modified to diminish outlying points that do not accurately represent the analyte concentration, for example due to signal noise or other signal artifacts, such as described in U.S. Pat. No. 6,931,327 to Goode et al., which is incorporated herein by reference in its entirety.

When a sensor is first implanted into host tissue, the sensor and receiver are initialized. This can be referred to as start-up mode and involves optionally resetting the sensor data and calibrating the sensor. In selected examples, mating the electronics unit to the mounting unit triggers a start-up mode. In other examples, the start-up mode is triggered by the receiver.

### Receiver

In some examples, the sensor electronics are wirelessly connected to a receiver via one- or two-way RF transmissions or the like. However, a wired connection is also contemplated. The receiver provides much of the processing and display of the sensor data and can be selectively worn and/or removed at the host's convenience. Thus, the sensor system can be discreetly worn, and the receiver, which provides much of the processing and display of the sensor data, can be selectively worn and/or removed at the host's convenience. Particularly, the receiver includes programming for retrospectively and/or prospectively initiating a calibration, converting sensor data, updating the calibration, evaluating received reference and sensor data, and evaluating the calibration for the analyte sensor, such as described in more detail with reference to U.S. Pat. No. 7,778,680 to Goode et al.

U.S. Pat. No. 7,134,999 to Brauker et al. describes systems and methods suitable for the sensor body and is incorporated herein by reference in its entirety. In examples, a biointerface membrane is formed onto the sensing mechanism as described in more detail elsewhere herein. The sensor body includes sensor electronics and communicates with a receiver as described in more detail, above. A drug releasing membrane can be disposed on at least a portion of biointerface membrane and/or sensing mechanism.

In certain examples, the sensing device, which is implantable into the host, such as in the soft tissue beneath the skin, is implanted subcutaneously, such as in the abdomen of the host, for example. A person of ordinary skill in the art appreciates a variety of suitable implantation sites available due to the sensor's small size. In some examples, the sensor architecture is less than about 0.5 mm in at least one dimension, for example a wire-based sensor with a diameter of less than about 0.5 mm. In another exemplary example, for example, the sensor may be 0.5 mm thick, 3 mm in length and 2 cm in width, such as possibly a narrow substrate, needle, wire, rod, sheet, or pocket. In another exemplary example, a plurality of about 1 mm wide wires about 5 mm in length could be connected at their first ends, producing a forked sensor structure. In still another example, a 1 mm wide sensor could be coiled, to produce a substantially planar, spiraled sensor structure. Although a few examples are cited above, numerous other useful examples are contemplated by the present disclosure, as is appreciated by a person of ordinary skill in the art.

Post implantation, a period of time is allowed for tissue ingrowth within the biointerface membrane. The length of time required for tissue ingrowth varies from host to host, such as about a week to about 3 weeks, although other time periods are also possible. Once a mature bed of vascularized tissue has grown into the biointerface membrane, a signal can be detected from the sensor, as described elsewhere herein and in U.S. Pat. Appl. Pub. No. 2005/0245799 to Brauker et al., which is incorporated herein in its entirety. Long term sensors can remain implanted and produce glucose signal information from months to years, as described in the above-cited patent application.

In certain examples, the device is configured such that the sensing unit is separated from the electronics unit by a tether or cable, or a similar structure. A person of ordinary skill in the art will recognize that a variety of known and useful means may be used to tether the sensor to the electronics. While not wishing to be bound by theory, it is believed that the FBR to the electronics unit alone may be greater than the FBR to the sensing unit alone, due to the electronics unit's greater mass, for example. Accordingly, separation of the sensing and electronics units effectively reduces the FBR to the sensing unit and results in improved device function. As described elsewhere herein, the architecture and/or composition of the sensing unit (e.g., inclusion of a drug releasing membrane with certain bioactive agents) can be implemented to further reduce the foreign body response to the tethered sensing unit.

In another example, an analyte sensor is designed with separate electronics and sensing units, wherein the sensing unit is inductively coupled to the electronics unit. In this example, the electronics unit provides power to the sensing unit and/or enables communication of data therebetween.

In yet another example, the implanted sensor additionally includes a capacitor to provide necessary power for device function. In examples, a portable scanner (e.g., wand-like device) is used to collect data stored on the circuit and/or to recharge the device.

In general, inductive coupling, as described herein, enables power to be transmitted to the sensor for continuous power, recharging, and the like. Additionally, inductive coupling utilizes appropriately spaced and oriented antennas (e.g., coils) on the sensing unit and the electronics unit so as to efficiently transmit/receive power (e.g., current) and/or data communication therebetween. One or more coils in each of the sensing and electronics unit can provide the necessary power induction and/or data transmission.

### Sterilization

In examples, the devices disclosed herein are sterilized. In examples, the devices disclosed herein are sterilized using high energy radiation, for example, e-beam, x-ray, or ultraviolet light. In another example, the devices disclosed herein are sterilized using ethylene oxide.

The above description discloses several methods and materials of the present disclosure. This disclosure is susceptible to modifications in the methods and materials, as well as alterations in the fabrication methods and equipment. Such modifications will become apparent to a person of ordinary skill in the art from a consideration of this disclosure or practice of the disclosure disclosed herein. Consequently, it is not intended that this disclosure be limited to the specific examples disclosed herein, but that it covers all modifications and alternatives coming within the true scope and spirit of the disclosure.

While certain examples of the present disclosure have been illustrated with reference to specific combinations of elements, various other combinations may also be provided without departing from the teachings of the present disclosure. Thus, the present disclosure should not be construed as being limited to the particular exemplary examples described herein and illustrated in the Figures, but may also encompass combinations of elements of the various illustrated examples and aspects thereof.

1. A device for continuous measurement of a concentration of an analyte, the device comprising:
a sensor configured to generate a signal associated with the concentration of the analyte;
a layer located over the sensor, the layer comprising a polyurethane, a poly(urea-urethane), or a poly(thiourethane-urethane), the polyurethane, the poly(urea-urethane), or the poly(thiourethane-urethane) comprising a repeating structure of:
[{soft segment}-{hard segment}]; or
[{soft segment}-{hard segment-chain extender-hard segment}]; and
wherein the soft segment comprises a backbone, the backbone having at least one polymer, at least one oligomer, or at least one monomer grafted thereto.

2. The device of claim 1, wherein the polyurethane, the poly(urea-urethane), or the poly(thiourethane-urethane), comprises the following Structure (I): 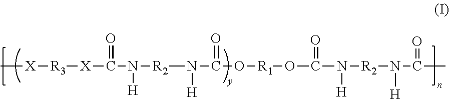
where n is nonzero; y is nonzero and represents hard segment-chain extender moles;
where each X is, independently, oxygen, nitrogen, or sulfur;
where R1 is a linear or branched alkyl, a polyether, a polyester, a polycarbonate, or is represented by Structure (III): 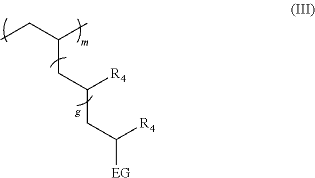
where m and g are nonzero; EG is H, an alkyl, aryl, benzyl, alkoxyl or hydrogen end group; R4 comprises furanyl, tetrahydrofuranyl, thiophenyl, pyrrolyl, pyrrolidinyl, pyranyl, pyridinyl, pyrazinyl, pyrimidinyl, pyrrolidonyl, piperidinyl, imidazolyl, thiazolyl, dioxanyl, morpholinyl, pyrimidinyl, isoxazolyl, oxazolyl, indolyl, isoindolyl, quinolinyl, isoquinolinyl, purinyl, carbazolyl, dibenzofuranyl, chromenyl, xanthenyl, or combinations thereof;
R2 is a residue of an organic polyisocyanate;
R3 is a linear aliphatic hydrocarbon, a non-linear aliphatic hydrocarbon, a cyclic aliphatic hydrocarbon, an aromatic hydrocarbon, or combinations thereof;
where at least one of R1 or R3 is represented by Structure (II): 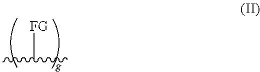
where [symbol] is the same as R1, where g is nonzero; and
FG is a functional group moiety that is the same or different when both R1 and R3 are represented by Structure (II).

3. The device of claim 1, wherein the at least one polymer, the at least one oligomer, or the at least one monomer comprises a zwitterionic group.

4. The device of claim 1, wherein the at least one polymer, the at least one oligomer, or the at least one monomer comprises a heterocycle.

5. The device of claim 4, wherein the heterocycle comprises nitrogen.

6. The device of claim 4, wherein the heterocycle comprises oxygen.

7. The device of claim 6, wherein the heterocycle comprises nitrogen and oxygen.

8. The device of claim 4, wherein the heterocycle is at least one of furanyl, tetrahydrofuranyl, thiophenyl, pyrrolyl, pyrrolidinyl, pyranyl, pyridinyl, pyrazinyl, pyrimidinyl, pyrrolidonyl, piperidinyl, imidazolyl, thiazolyl, dioxanyl, morpholinyl, pyrimidinyl, isoxazolyl, oxazolyl, indolyl, isoindolyl, quinolinyl, isoquinolinyl, purinyl, carbazolyl, dibenzofuranyl, chromenyl, and xanthenyl.

9. The device of claim 2, wherein the functional group moiety is a grafted monomer selected from the group: N-vinylpyrrolidone monomer; vinyl pyridine monomer; vinyl pyrrole monomer, vinyl imidazole monomer, vinyl pyrimidine monomer, vinyl acetate monomer; acrylonitrile monomer; maleic anhydride monomer, poly(ethylene glycol) methyl ether methacrylate monomer; 2-(dimethylamino)ethyl methacrylate monomer; or combinations thereof.

10. The device of claim 1, wherein the polyurethane, the poly(urea-urethane), or the poly(thiourethane-urethane) is configured to control a flux of at least one analyte in vivo and configured to control a flux of at least one interferent in vivo.

11. The device of claim 1, wherein the layer is adjacent an electrode surface.

12. The device of claim 1, wherein the layer is directly adjacent an electrode surface.

13. The device of claim 1, wherein the layer is adjacent an enzyme-containing layer.

14. The device of claim 1, wherein the layer is directly adjacent an enzyme-containing layer.

15. The device of claim 1, wherein the layer is between an electrode surface and an enzyme-containing layer.

16. The device of claim 1, wherein the layer is between an enzyme-containing layer and a biointerface layer.

17. The device of claim 1, wherein the analyte is glucose.

18. The device of claim 1, wherein the analyte is a ketone.

19. The device of claim 10, wherein the at least one interferent is acetaminophen or a metabolite thereof.

20-84. (canceled)

85. The device of claim 1, wherein the analyte is a lactate.
